SEQUENCE LISTING (62 sequences)

SEQ ID NO: 1 - protein, 107 aa, Homo sapiens [sequence omitted]

SEQ ID NO: 2 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPSK

SEQ ID NO: 4 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 327 aa, Artificial Sequence
  Synthetic Polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 7 - protein, 107 aa, Artificial Sequence [sequence omitted]

SEQ ID NO: 8 - protein, 120 aa, Artificial Sequence [sequence omitted]

SEQ ID NO: 9 - protein, 214 aa, Artificial Sequence
  Synthetic Polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 450 aa, Artificial Sequence
  Synthetic Polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 447 aa, Artificial Sequence
  Synthetic Polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Artificial Sequence [sequence omitted]

SEQ ID NO: 13 - protein, 119 aa, Artificial Sequence [sequence omitted]

SEQ ID NO: 14 - protein, 107 aa, Artificial Sequence
  Synthetic Polypeptide
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 15 - protein, 120 aa, Artificial Sequence
  Synthetic Polypeptide
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 16 - protein, 5 aa, Artificial Sequence
  Synthetic Polypeptide
DTYIH

SEQ ID NO: 17 - protein, 17 aa, Artificial Sequence
  Synthetic Polypeptide
RIYPTNGYTRYADSVKG

SEQ ID NO: 18 - protein, 11 aa, Artificial Sequence
  Synthetic Polypeptide
WGGDGFYAMDY

SEQ ID NO: 19 - protein, 11 aa, Artificial Sequence
  Synthetic Polypeptide
RASQDVNTAVA

SEQ ID NO: 20 - protein, 7 aa, Artificial Sequence
  Synthetic Polypeptide
SASFLYS

SEQ ID NO: 21 - protein, 9 aa, Artificial Sequence
  Synthetic Polypeptide
QQHYTTPPT

SEQ ID NO: 22 - protein, 5 aa, Artificial Sequence
  Synthetic Polypeptide
DYTMD

SEQ ID NO: 23 - protein, 17 aa, Artificial Sequence
  Synthetic Polypeptide
DVNPNSGGSIYNQRFKG

SEQ ID NO: 24 - protein, 10 aa, Artificial Sequence
  Synthetic Polypeptide
NLGPSFYFDY

SEQ ID NO: 25 - protein, 11 aa, Artificial Sequence
  Synthetic Polypeptide
KASQDVSIGVA

SEQ ID NO: 26 - protein, 7 aa, Artificial Sequence
  Synthetic Polypeptide
SASYRYT

SEQ ID NO: 27 - protein, 9 aa, Artificial Sequence
  Synthetic Polypeptide
QQYYIYPYT

SEQ ID NO: 28 - protein, 5 aa, Artificial Sequence
  Synthetic Polypeptide
DTYIH

SEQ ID NO: 29 - protein, 17 aa, Artificial Sequence
  Synthetic Polypeptide
RIYPTNGYTRYDPKFQD

SEQ ID NO: 30 - protein, 11 aa, Artificial Sequence
  Synthetic Polypeptide
WGGDGFYAMDY

SEQ ID NO: 31 - protein, 11 aa, Artificial Sequence
  Synthetic Polypeptide
KASQDVNTAVA

SEQ ID NO: 32 - protein, 7 aa, Artificial Sequence
  Synthetic Polypeptide
SASFRYT

SEQ ID NO: 33 - protein, 9 aa, Artificial Sequence
  Synthetic Polypeptide
QQHYTTPPT

SEQ ID NO: 34 - protein, 25 aa, Artificial Sequence
  Synthetic Polypeptide
LLNNFYPREAKVCWKVDNALQSGNS

SEQ ID NO: 35 - protein, 25 aa, Artificial Sequence
  Synthetic Polypeptide
SSTLTLSKADYECHKVYACEVTHQG

SEQ ID NO: 36 - protein, 25 aa, Artificial Sequence
  Synthetic Polypeptide
KHKVYACEVTHQCLSSPVTKSFNRG

SEQ ID NO: 37 - protein, 25 aa, Artificial Sequence
  Synthetic Polypeptide
HKVYACEVTHQGCSSPVTKSFNRGE

SEQ ID NO: 38 - protein, 20 aa, Artificial Sequence
  Synthetic Polypeptide
EVTHQGLSSPCTKSFNRGEC

SEQ ID NO: 39 - protein, 21 aa, Artificial Sequence
  Synthetic Polypeptide
CEVTHQGLSSPVCKSFNRGEC

SEQ ID NO: 40 - protein, 25 aa, Artificial Sequence
  Synthetic Polypeptide
GCLVKDYFPEPVCVSWNSGALTSGV

SEQ ID NO: 41 - protein, 25 aa, Artificial Sequence
  Synthetic Polypeptide
LVKDYFPEPVTVCWNSGALTSGVHT

SEQ ID NO: 42 - protein, 25 aa, Artificial Sequence
  Synthetic Polypeptide
PVTVSWNSGALTCGVHTFPAVLQSS

SEQ ID NO: 43 - protein, 25 aa, Artificial Sequence
  Synthetic Polypeptide
SWNSGALTSGVHCFPAVLQSSGLYS

SEQ ID NO: 44 - protein, 25 aa, Artificial Sequence
  Synthetic Polypeptide
VVTVPSSSLGTQCYICNVNHKPSNT

SEQ ID NO: 45 - protein, 25 aa, Artificial Sequence
  Synthetic Polypeptide
VVTVPSSNFGTQCYTCNVDHKPSNT

SEQ ID NO: 46 - protein, 25 aa, Artificial Sequence
  Synthetic Polypeptide
VVTVPSSSLGTQCYTCNVNHKPSNT

SEQ ID NO: 47 - protein, 25 aa, Artificial Sequence
  Synthetic Polypeptide
VVTVPSSSLGTKCYTCNVDHKPSNT

SEQ ID NO: 48 - protein, 23 aa, Artificial Sequence
  Synthetic Polypeptide
TVPSSSLGTQTYCCNVNHKPSNT

SEQ ID NO: 49 - protein, 23 aa, Artificial Sequence
  Synthetic Polypeptide
TVPSSNFGTQTYCCNVDHKPSNT

SEQ ID NO: 50 - protein, 23 aa, Artificial Sequence
  Synthetic Polypeptide
TVPSSSLGTQTYCCNVNHKPSNT

SEQ ID NO: 51 - protein, 23 aa, Artificial Sequence
  Synthetic Polypeptide
TVPSSSLGTKTYCCNVDHKPSNT

SEQ ID NO: 52 - protein, 25 aa, Artificial Sequence
  Synthetic Polypeptide
TYICNVNHKPSNCKVDKKVEPKSCD

SEQ ID NO: 53 - protein, 25 aa, Artificial Sequence
  Synthetic Polypeptide
TYTCNVDHKPSNCKVDKTVERKCCV

SEQ ID NO: 54 - protein, 25 aa, Artificial Sequence
  Synthetic Polypeptide
TYTCNVNHKPSNCKVDKRVELKTPL

SEQ ID NO: 55 - protein, 25 aa, Artificial Sequence
  Synthetic Polypeptide
TYTCNVDHKPSNCKVDKRVESKYGP

SEQ ID NO: 56 - protein, 25 aa, Artificial Sequence
  Synthetic Polypeptide
ICNVNHKPSNTKCDKKVEPKSCDKT

SEQ ID NO: 57 - protein, 25 aa, Artificial Sequence
  Synthetic Polypeptide
TCNVDHKPSNTKCDKTVERKCCVEC

SEQ ID NO: 58 - protein, 25 aa, Artificial Sequence
  Synthetic Polypeptide
TCNVNHKPSNTKCDKRVELKTPLGD

SEQ ID NO: 59 - protein, 25 aa, Artificial Sequence
  Synthetic Polypeptide
TCNVDHKPSNTKCDKRVESKYGPPC

SEQ ID NO: 60 - protein, 18 aa, Artificial Sequence
  Synthetic Polypeptide
EALHNHYTQKSLCLSPGK

SEQ ID NO: 61 - protein, 18 aa, Artificial Sequence
  Synthetic Polypeptide
EALHNRFTQKSLCLSPGK

SEQ ID NO: 62 - protein, 18 aa, Artificial Sequence
  Synthetic Polypeptide
EALHNHYTQKSLCLSLGK

(19) United States
(12) Patent Application Publication
(10) Pub. No.: US 2016/0051695 A1
(43) Pub. Date: Feb. 25, 2016

(54) HER2 ANTIBODY-DRUG CONJUGATES

(71) Applicants: AbGenomics International Inc., Dover, DE (US); BioAlliance C.V., Alkmaar (NL)

(72) Inventors: Rong-Hwa Lin, Palo Alto, CA (US); Shih-Yao Lin, Taipei (TW); Yu-Chi Hsieh, New Taipei City 221 (TW); Chiu-Chen Huang, Taipei (TW); Shu-Hua Lee, Taipei (TW); Yu-Ying Tsai, Taipei City 116 (TW)

(21) Appl. No.: 14/743,351

(22) Filed: Jun. 18, 2015

Related U.S. Application Data
(60) Provisional application No. 62/015,661, filed on Jun. 23, 2014.
(60) Provisional application No. 62/014,912, filed on Jun. 20, 2014.

(51) Int. Cl. A61K 47/48

(52) U.S. Cl. CPC A61K 47/48569; A61K 47/48715; A61K 47/48415

(57) ABSTRACT

The present disclosure provides compounds with a hydrophilic self-immolative linker, which is cleavable under appropriate conditions and incorporates a hydrophilic group to provide better solubility of the compound. The compounds of the present disclosure comprise a drug moiety, a targeting moiety capable of targeting a selected cell population, and a linker which contains an acyl unit, an optional spacer unit for providing distance between the drug moiety and the targeting moiety, a peptide linker which can be cleaved under appropriate conditions, a hydrophilic self-immolative linker, and an optional second self-immolative spacer or cyclization self-elimination linker. In some aspects of the present disclosure, the targeting moiety is an anti-HER2 antibody. The present disclosure further provides compositions and methods for treating cancers.

### RELATED APPLICATIONS

The present application claims priority under 35 U.S.C. §119(e) to U.S. provisional patent applications, U.S. Ser. No. 62/015,661, filed Jun. 23, 2014, and U.S. Ser. No. 62/014,912, filed Jun. 20, 2014, each which is incorporated herein by reference.

### FIELD OF INVENTION

The present disclosure is in the field of anti-cancer therapeutics, and provides efficacy and specificity for the delivery of cytotoxic drugs specifically to cancer cells through an antibody-drug conjugate (ADC) format.

### BACKGROUND

Antibody-drug conjugates (ADCs) are a class of therapeutics that combine the specificity of monoclonal antibodies (mAbs) with the potency of cytotoxic molecules. The use of ADC empowers the cancer killing activity of antibody by conjugated cytotoxic agents, while target-specific delivery avoids systemic toxicity caused by exposure to free toxic agents. Currently, two ADCs have been approved by FDA for treating human cancers. ADCETRIS® (Brentuximab vedotin or SGN-35), an anti-CD30 antibody conjugated with cytotoxic agent MMAE, is designed to treat CD30-positive relapsing lymphoma. KADCYLA® (T-DM1), an anti-HER2 antibody conjugated with cytotoxic agent DM1, is designed to treat HER2-positive metastatic breast cancer.

Linker technology profoundly impacts ADC potency, specificity, and safety. Enzyme-labile linkers utilize the differential activities of proteases inside and outside of the cells to achieve control of the drug release. A drug can be conjugated to antibody via peptide bond, and can only be specifically cleaved by the action of lysosomal proteases present inside the cells, and at elevated levels in certain tumor types (Koblinsk et al, 2000). This will ensure the stability of linker in the blood stream to limit the damage to healthy tissue. However, the increased associated hydrophobicity of some enzyme-labile linkers can lead to aggregation of ADC, particularly with strongly hydrophobic drugs. Thus, there is a need for linkers which can provide serum stability, as well as increased solubility, allowing efficient conjugation and intracellular delivery of hydrophobic drugs.

The human epidermal growth factor receptor 2 protein, HER2 (ErbB2), is a member of the epidermal growth factor receptor family. These receptor tyrosine kinases are known to play critical roles in both development and oncogenesis. Overexpression of HER2 protein is observed in 25%-30% of primary breast cancers (Press et al, 1993), and thus becomes an important candidate of cancer targeting therapy. The humanized anti-HER2 antibody, trastuzumab, has been shown, both in vitro and in mouse xenograft models, to inhibit the proliferation of human tumor cells that overexpress HER2 (Hudziak et al, 1989; Baselga et al, 1998). Although trastuzumab is clinically active and showed efficacy in treating patients with HER2-overexpressing metastatic breast cancers (Baselga et al, 1996), the majority of this population who initially responded to trastuzumab developed resistance within one year (Romond et al, 2005; Nahta et al, 2006; Pohlmann et al, 2009). Accordingly, there is a need to develop novel therapies against tumor cells that overexpress HER2.

### SUMMARY

The compounds of the present disclosure comprise a drug moiety, a targeting moiety capable of targeting a selected cell population, and a linker which contains an acyl unit, an optional spacer unit for providing distance between the drug moiety and the targeting moiety, a peptide linker which can be cleavable under appropriate conditions, a hydrophilic self-immolative linker, and an optional second self-immolative spacer or cyclization self-elimination linker.

The present disclosure also provides a compound of Formula (I):

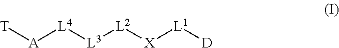

or a salt or solvate or stereoisomer thereof;
wherein:
D is a drug moiety;
T is a targeting moiety;
X is a hydrophilic self-immolative linker;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit.

The present disclosure also provides a compound of Formula (II):

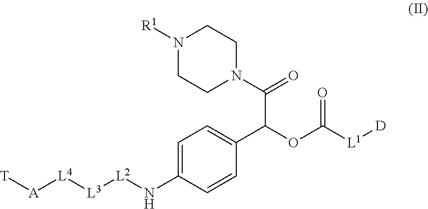

or a salt or solvate or stereoisomer thereof;
wherein:
D is a drug moiety;
T is a targeting moiety;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond, a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit.

In some embodiments, a compound of Formula (Ia) is provided:

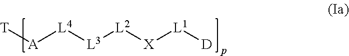

or a salt or solvate or stereoisomer thereof; wherein D, T, X, L¹, L², L³, L⁴ and A are as defined for Formula (I), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In some embodiments, a compound of Formula (IIa) is provided:

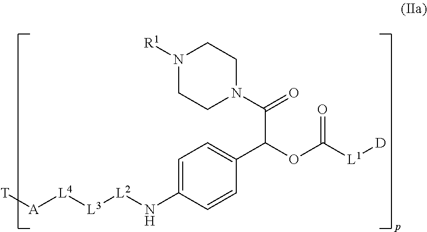

or a salt or solvate or stereoisomer thereof; wherein D, T, L¹, L², L³, L⁴ and A are as defined for Formula (II), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In some embodiments of the compounds of Formulae I, II, Ia and IIa, T is an antibody targeting molecule. In some embodiments, T is an anti-HER2 antibody. In some embodiments T is a monoclonal anti-HER2 antibody. In some embodiments the monoclonal anti-HER2 antibody is pertuzumab. In some embodiments, the monoclonal anti-HER2 antibody is margetuximab. In some embodiments, T is a humanized anti-HER2 antibody. In some embodiments, the humanized anti-HER2 antibody is trastuzumab.

In some embodiments of the compounds of Formulae I, II, Ia and IIa, one or more amino acid residues of the heavy chain and/or the light chain of the antibody is replaced with a cysteine residue. In some embodiments, one or more amino acid residues of the Fc region of the antibody is replaced with a cysteine residue. In some embodiments, one or more amino acid residues of the antibody is replaced with a cysteine residue at position 147, 188, 200, 201 and/or 206 of the light chain, and/or at position 155, 157, 165, 169, 197, 199, 209, 211 and/or 442 of the heavy chain using EU numbering (EU index in Kabat). In some embodiments, D is linked to T by way of the cysteine residue. In some embodiments, D is an amino group-containing drug moiety, wherein the drug is connected to L¹ or X through the amino group of the amino group-containing drug moiety. In some embodiments, D is duocarmycin, dolastatin, tubulysin, doxorubicin (DOX), paclitaxel, or mitomycin C (MMC), or an amino derivative thereof.

In any of the embodiments described above, A-L⁴-L³-L²-X-L¹-D is:

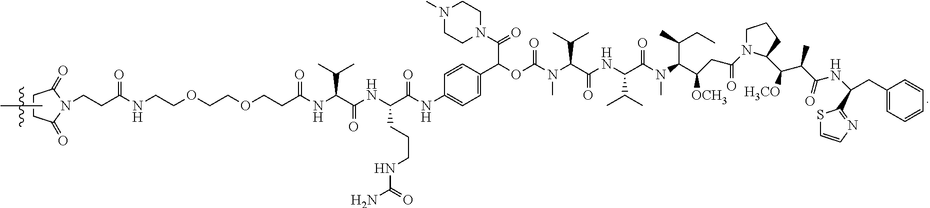

Some aspects of the disclosure involve a pharmaceutical composition comprising a compound described herein, or a salt or solvate or stereoisomer thereof; and a pharmaceutically acceptable carrier.

Some aspects of the disclosure involve a method of killing a cell. The method comprises administering to the cell an amount of a compound described herein, or a salt or solvate or stereoisomer or a pharmaceutical composition thereof, sufficient to kill the cell. In some embodiments, the cell is a cancer cell. In some embodiments, the cancer cell is a breast cancer cell, gastric cancer cell, or ovarian cancer cell.

Some aspects of the disclosure involve a method of treating cancer in an individual in need thereof. The method comprises administering to the individual an effective amount of a compound of described herein, or a salt or solvate or stereoisomer or a pharmaceutical composition thereof. In some embodiments, the cancer is breast cancer, gastric cancer, or ovarian cancer.

Some aspects of the disclosure involve a compound of Formula I, Ia, II or IIa or a salt or solvate or stereoisomer or a pharmaceutical composition thereof for use in treating cancer. In some embodiments, the cancer is breast cancer, gastric cancer, or ovarian cancer.

In some embodiments, L¹ is a bond. In some embodiments, L¹ is a self-immolative linker. In some embodiments, L¹ is an aminobenzyloxycarbonyl linker. In some embodiments, L¹ is selected from the group consisting of

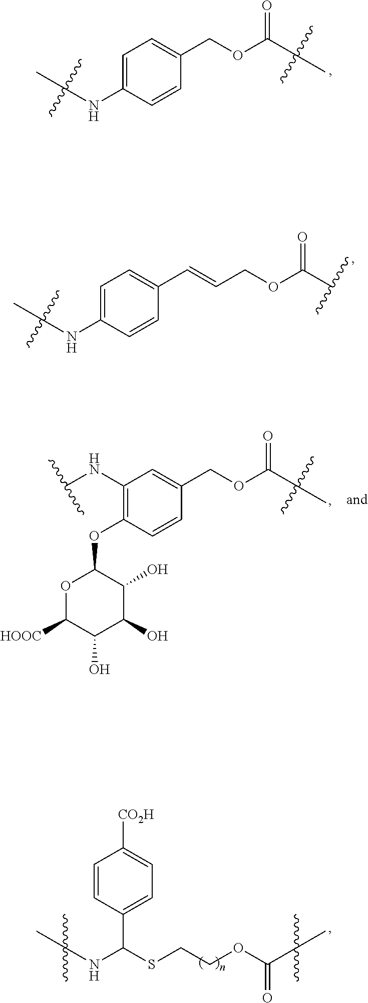

wherein n is 1 or 2.

In some embodiments, L¹ is selected from the group consisting of

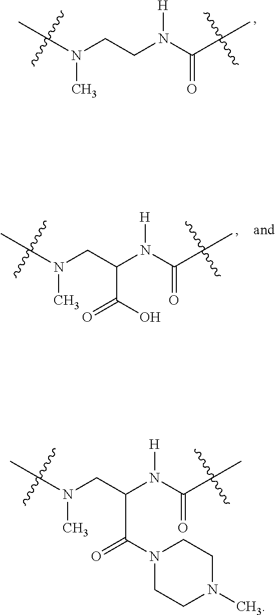

In some embodiments, L² is a bond. In some embodiments, L² is a self-immolative linker. In some embodiments, L² is an aminobenzyloxycarbonyl linker. In some embodiments, L² is selected from

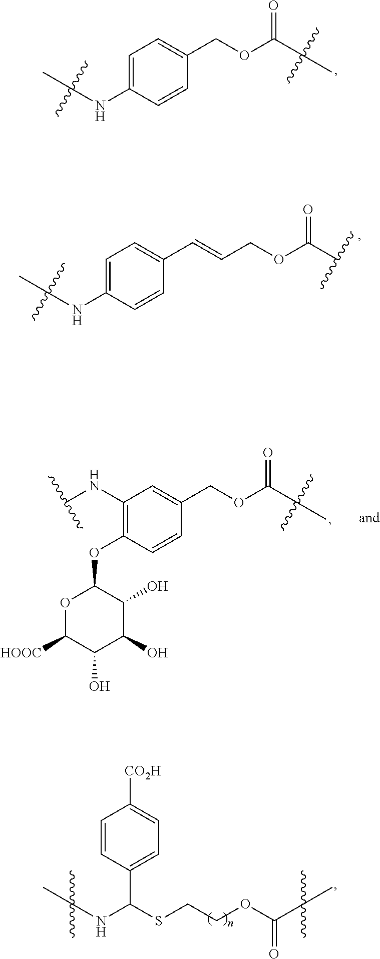

wherein n is 1 or 2.

In some embodiments, L³ is a peptide linker of 1 to 10 amino acid residues. In some embodiments, L³ is a peptide linker of 2 to 4 amino acid residues. In some embodiments, L³ is a peptide linker of 1, 2, 3, 4, 5, 6, 7, 8, 9, or 10 amino acid residues. In some embodiments, L³ is a peptide linker comprising at least one lysine or at least one arginine residue. In some embodiments, L³ is a peptide linker comprising an amino acid residue selected from lysine, D-lysine, citrulline, arginine, proline, histidine, ornithine and glutamine. In some embodiments, L³ is a peptide linker comprising an amino acid residue selected from valine, isoleucine, phenylalanine, methionine, asparagine, proline, alanine, leucine, tryptophan, and tyrosine. In some embodiments, L³ is a dipeptide unit selected from valine-citrulline, proline-lysine, methionine-D-lysine, asparagine-D-lysine, isoleucine-proline, phenylalanine-lysine, and valine-lysine. In some embodiments, L³ is valine-citrulline.

In some embodiments, L⁴ is a bond. In some embodiments, L⁴ is a spacer. In some embodiments, the spacer is polyalkylene glycol, alkylene, alkenylene, alkynylene, or polyamine. In some embodiments, L⁴ is L^{4a}-C(O), L^{4a}-C(O)—NH, L^{4a}-S(O)₂, or L^{4a}-S(O)₂—NH, wherein each L^{4a} is independently polyalkylene glycol, alkylene, alkenylene, alkynylene, or polyamine. In some embodiments, L⁴ is L^{4a}-C(O), wherein L^{4a} is polyalkylene glycol, alkylene, alkenylene, alkynylene, or polyamine. In some embodiments, L⁴ is L^{4a}-C(O), wherein L^{4a} is a polyalkylene glycol. In some embodiments, L⁴ is L^{4a}-C(O), wherein L^{4a} is a polyethylene glycol. In some embodiments, the spacer is of the formula —CH₂—(CH₂—O—CH₂)ₘ—CH₂—C(O)—, wherein m is an integer from 0 to 30. In some embodiments, the spacer is of the formula —CH₂—(CH₂—O—CH₂)ₘ—CH₂—C(O)—, wherein m is the integer 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In some embodiments, L⁴ is L^{4a}-C(O), wherein L^{4a} is alkylene.

In some embodiments, A is selected from the group consisting of

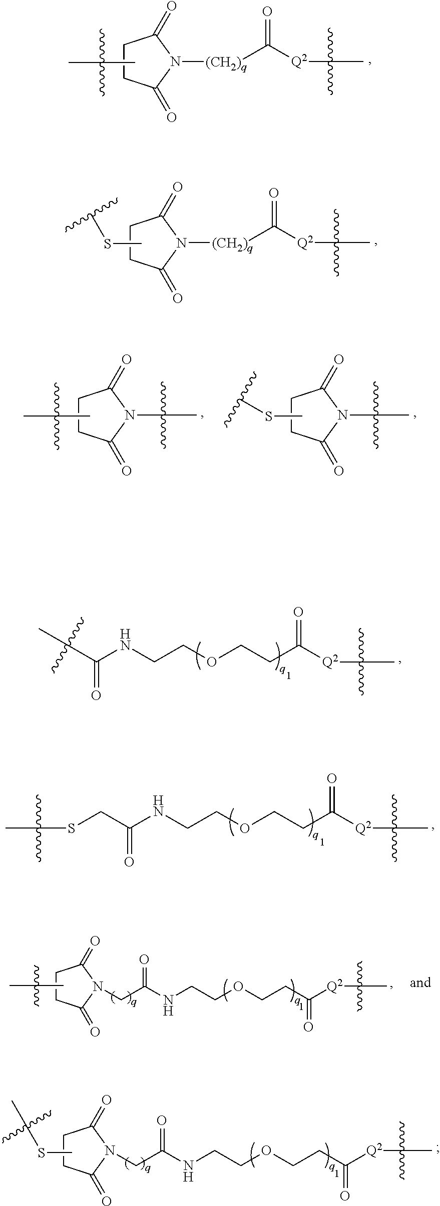

wherein each Q² is NH or O, and each q is an integer from 1 to 10, and each q₁ is independently an integer from 1 to 10. In some embodiments, q is 2, 3, 4, or 5. In certain embodiments, q₁ is 2, 3, 4, or 5. In further embodiments, each q is the integer 1, 2, 3, 4, 5, 6, 7, 8, 9, or 10. In some embodiments, A is

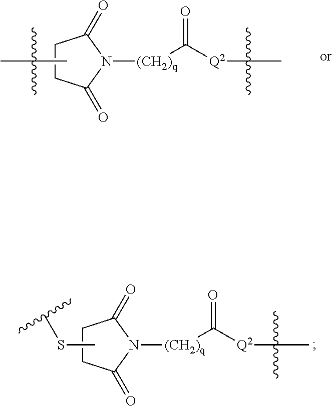

wherein each Q2 is independently NH or O and each q is independently an integer from 1 to 10. In further embodiments, each q is independently the integer 1, 2, 3, 4, 5, 6, 7, 8, 9, or 10. In some embodiments, q is 2, 3, 4, or 5. In some embodiments, A is selected from the group

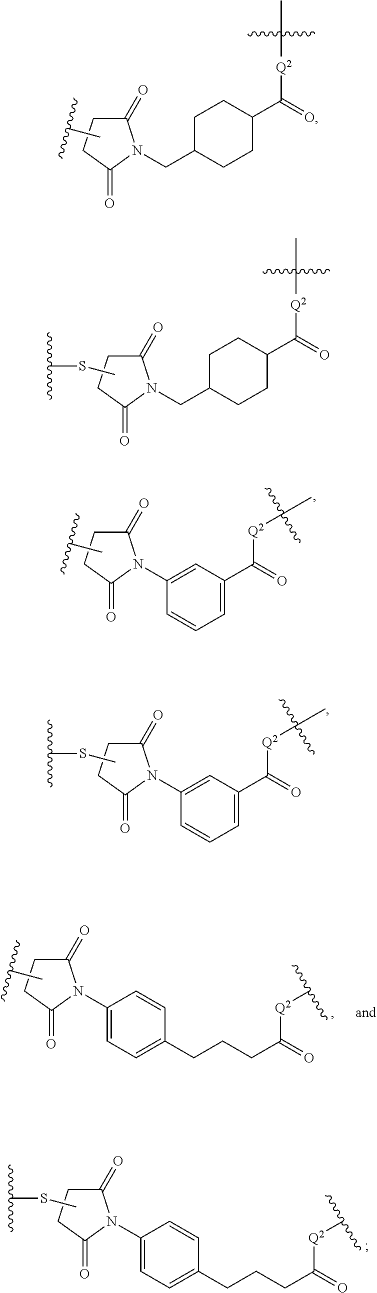

wherein each Q² is independently NH or 0.

In some embodiments, D is an amino group-containing drug moiety, wherein the drug is connected to L¹ or X through the amino group of the amino group-containing drug moiety. In some embodiments, D is an amino derivative of duocarmycin selected from the group consisting of:

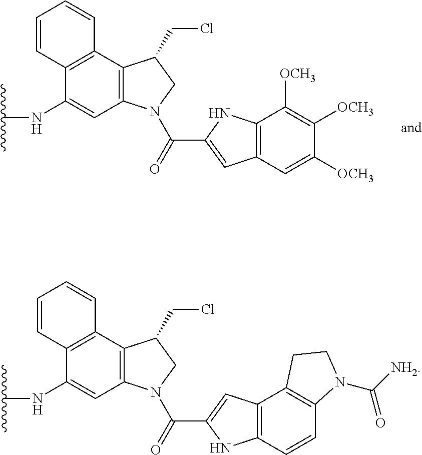 [SMILES: *Nc1cc2c(c3ccccc13)[C@H](CCl)CN2C(=O)c1cc2cc(OC)c(OC)c(OC)c2[nH]1 ; *Nc1cc2c(c3ccccc13)[C@H](CCl)CN2C(=O)c1cc2c3c(ccc2[nH]1)N(C(N)=O)CC3]

In some embodiments, D is an amino derivative of dolastatin (e.g. monomethyl Dolastatin 10):

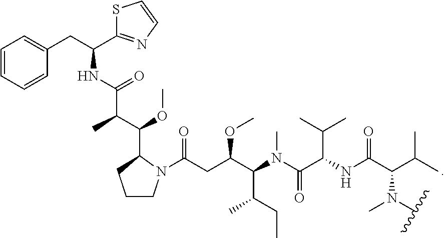 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C)C(C)C]

In some embodiments, A-L⁴-L³-L² is

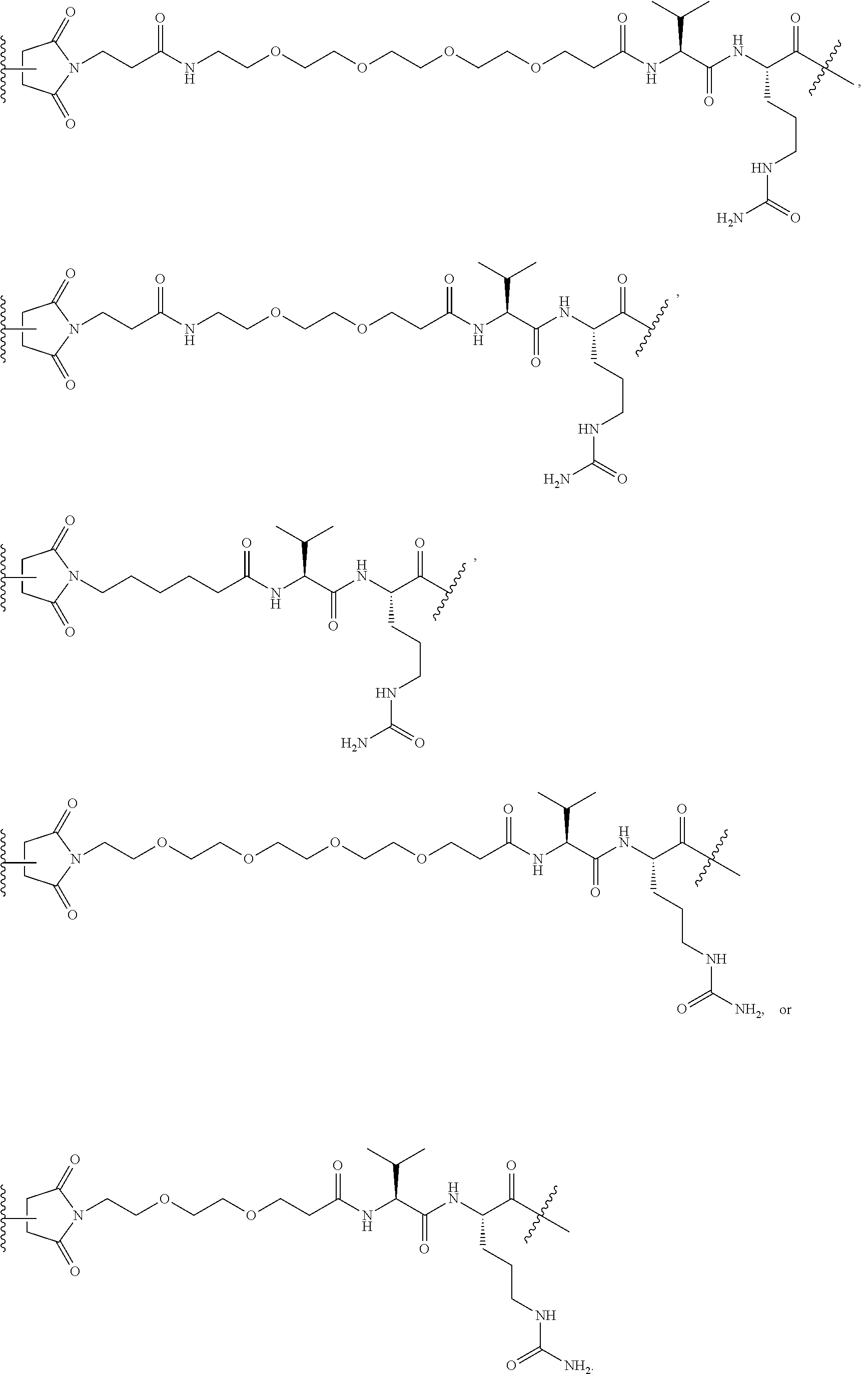

In some embodiments, A-L⁴-L³-L² is:

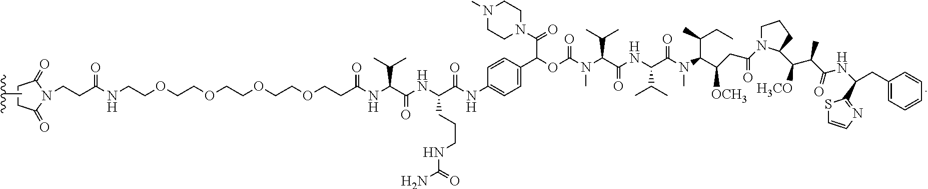

In some embodiments, A-L⁴-L³-L²-X-L¹-D is:

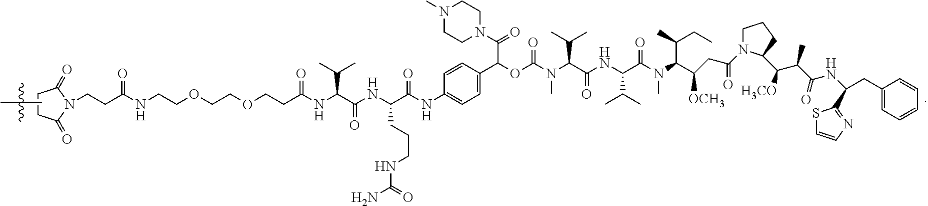

In some embodiments, A-L⁴-L³-L²-X-L¹-D is:

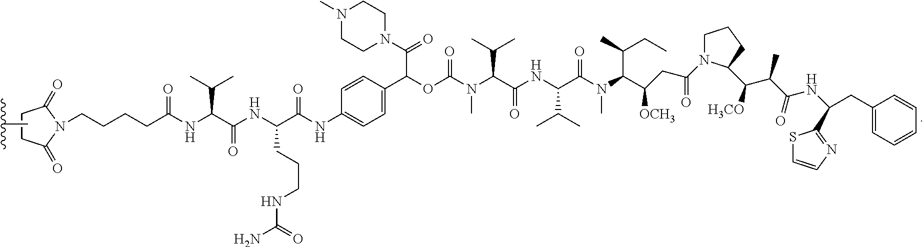

Some aspects of the disclosure involve a method of preparing a compound of formula (II):

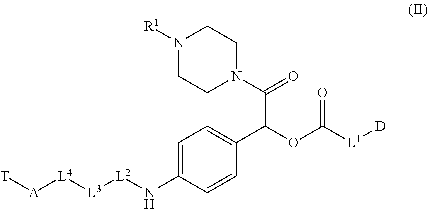

or a salt or solvate or stereoisomer thereof;
wherein:
D is a drug moiety;
T is an antibody;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond or a self-immolative linker;
   wherein if L¹ is a self-immolative linker or a cyclization self-elimination linker, then L² is a bond;
   wherein if L² is a self-immolative linker, then L¹ is a bond;
L³ is a peptide linker;
L⁴ is a bond or a spacer; and
A is an acyl unit. The method comprises reacting an antibody with Compound Z:

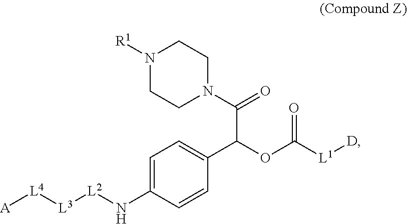

or a salt or solvate or stereoisomer thereof.

Some aspects of the disclosure involve a method of preparing a compound of formula (IIa):

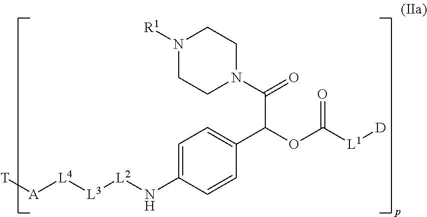

or a salt or solvate or stereoisomer thereof;
wherein:
p is 1, 2, 3, 4, 5, 6, 7, 8, 9, 10, 11, 12, 13, 14, 15, 16, 17, 18, 19, or 20;
D is a drug moiety;
T is an antibody;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond or a self-immolative linker;
   wherein if L¹ is a self-immolative linker or a cyclization self-elimination linker, then L² is a bond;
   wherein if L² is a self-immolative linker, then L¹ is a bond;
L³ is a peptide linker;
L⁴ is a bond or a spacer; and
A is an acyl unit. The method comprises reacting an antibody with Compound Z:

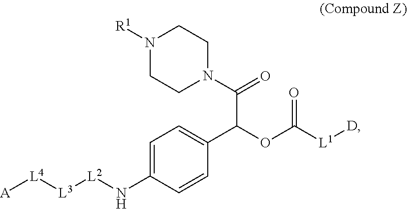

or a salt or solvate or stereoisomer thereof.

In some embodiments, the antibody is an anti-HER2 antibody. In some embodiments, the antibody is a monoclonal anti-HER2 antibody. In some embodiments, the antibody is a humanized anti-HER2 antibody, optionally a monoclonal humanized anti-HER2 antibody. In some embodiments, one or more amino acid residues of the antibody heavy chain and/or the light chain is replaced with a cysteine residue. In some embodiments, one or more amino acid residues of the Fc region of the antibody is replaced with a cysteine residue. In some embodiments, one or more amino acid residues of the antibody is replaced with a cysteine residue at position 147, 188, 200, 201 and/or 206 of the light chain, and/or at position 155, 157, 165, 169, 197, 199, 209, 211 and/or 442 of the heavy chain using EU numbering (EU index in Kabat). In some embodiments, the antibody comprises one or more sulfhydryl groups. In some embodiments, the compound is prepared using one of the methods described herein, wherein the antibody comprises one or more sulfhydryl groups.

Also provided are pharmaceutical compositions comprising a compound described herein, or a salt or solvate or stereoisomer thereof, and a pharmaceutically acceptable carrier.

Some aspects of the disclosure involve a compound of Formula (IX)

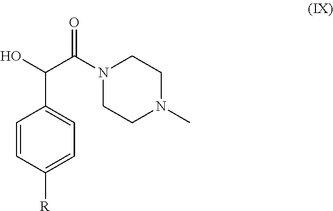

or a salt or solvate or stereoisomer thereof; wherein R is NO₂ or NH₂.

Some aspects of the disclosure involve a method of preparing Compound X:

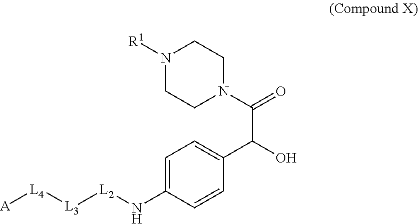

or a salt or solvate or stereoisomer thereof;
wherein:
L² is a bond or a self-immolative linker;
L³ is a peptide linker;
L⁴ is a bond or a spacer; and
A is an acyl unit; and
R¹ is hydrogen, unsubstituted or substituted C₁₋₃ alkyl, or unsubstituted or substituted heterocyclyl. The method comprises reacting Compound W: A-L⁴-L³-L²; and Compound I:

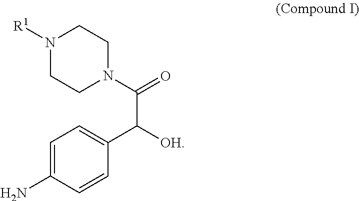

Some aspects of the disclosure involve a method of preparing Compound Z:

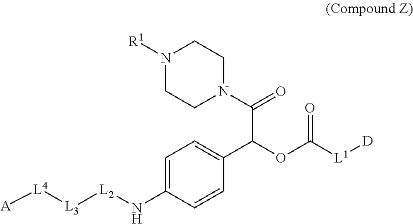

or a salt or solvate or stereoisomer thereof;
wherein:
D is a drug moiety;
L¹ is a bond, a self-immolative linker, or a cyclization self-elimination linker;
L² is a bond or a self-immolative linker;
   wherein if L¹ is a self-immolative linker or a cyclization self-elimination linker, then L² is a bond;
   wherein if L² is a self-immolative linker, then L¹ is a bond; L³ is a peptide linker;
L⁴ is a bond or a spacer; and
A is an acyl unit
R¹ is hydrogen, unsubstituted or substituted C₁₋₃ alkyl, or unsubstituted or substituted heterocyclyl.

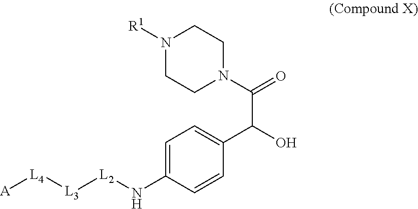

The method comprises: reacting Compound X:
and p-nitrophenylchloroformate to form Compound Y:

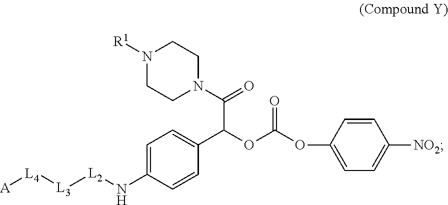

and reacting Compound Y with a compound comprising L¹-D.

Some aspects of the disclosure involve a method of preparing Compound X¹:

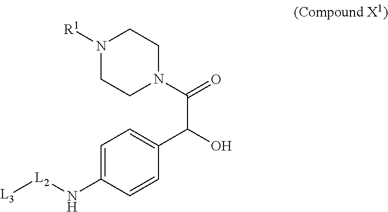

or a salt or solvate or stereoisomer thereof;
wherein:
L² is a bond or a self-immolative linker;
L³ is a peptide linker; and
R¹ is hydrogen, unsubstituted or substituted C₁₋₃ alkyl, or unsubstituted or substituted heterocyclyl. The method comprises: reacting Compound W¹: L³-L²; and Compound I:

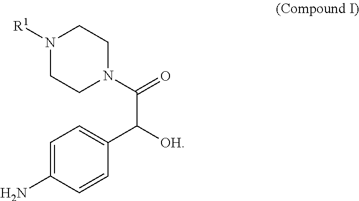

Some aspects of the disclosure involve a method of preparing Compound Y¹:

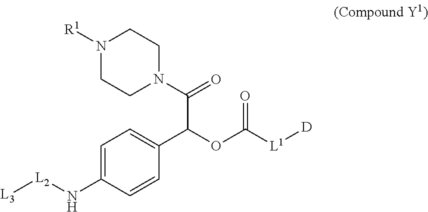

or a salt or solvate or stereoisomer thereof;
wherein:
D is drug moiety;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond; L³ is a peptide linker; and
R¹ is hydrogen, unsubstituted or substituted C₁₋₃ alkyl, or unsubstituted or substituted heterocyclyl. The method comprises: reacting Compound X¹:

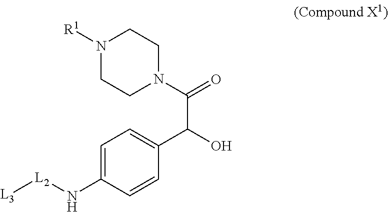

and a compound comprising L¹-D in the presence of p-nitrophenyl chloroformate. In some embodiments, a compound selected from the group consisting of bis(4/p-nitrophenyl) carbonate, phosgene, triphosgene/bis(trichloromethyl carbonate), trichloromethyl chloroformate, N,N′-disuccinimidyl carbonate, and 1,1′-carbonyldiimidazole, replaces p-nitrophenyl chloroformate in the method of preparing Compound Y¹.

Some aspects of the disclosure involve a method of preparing Compound Z:

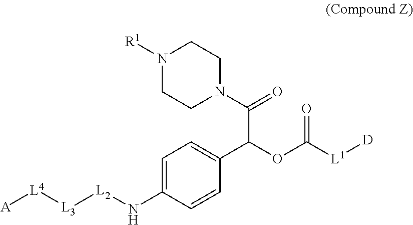

or a salt or solvate or stereoisomer thereof;
wherein:
D is a drug moiety;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is bond or a spacer;
A is an acyl unit; and
R¹ is hydrogen, unsubstituted or substituted C₁₋₃ alkyl, or unsubstituted or substituted heterocyclyl.

The method comprises: reacting Compound Y¹:

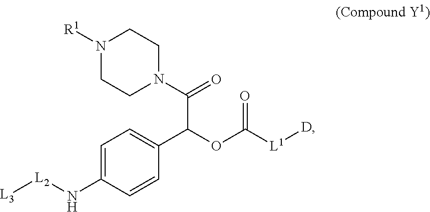

and a compound comprising A-L⁴.

Also provided is a compound of formula:

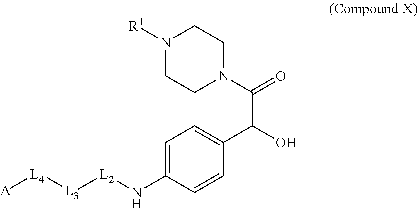

or a salt or solvate or stereoisomer thereof;
wherein:
L² is a bond or a self-immolative linker;
L³ is a peptide linker;
L⁴ is a bond or a spacer; and
A is an acyl unit; and
R¹ is hydrogen, unsubstituted or substituted C₁₋₃ alkyl, or unsubstituted or substituted heterocyclyl.

Also provided is a compound of formula:

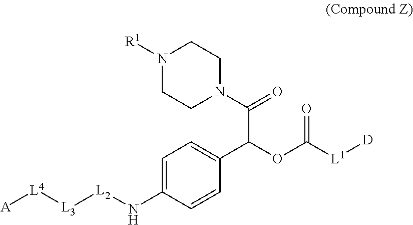

or a salt or solvate or stereoisomer thereof;
wherein:
D is a drug moiety;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit; and
R¹ is hydrogen, unsubstituted or substituted C₁₋₃ alkyl, or unsubstituted or substituted heterocyclyl.

Also provided is a compound of formula:

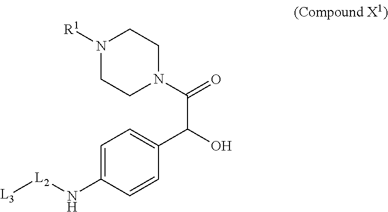

or a salt or solvate or stereoisomer thereof;
wherein:
L² is a bond or a self-immolative linker;
L³ is a peptide linker; and
R¹ is hydrogen, unsubstituted or substituted C₁₋₃ alkyl, or unsubstituted or substituted heterocyclyl.

Also provided is a compound of formula:

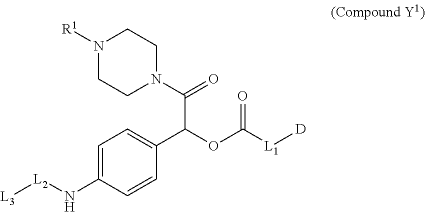

or a salt or solvate or stereoisomer thereof;
wherein:
D is a drug moiety;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker; and
R¹ is hydrogen, unsubstituted or substituted C₁₋₃ alkyl, or unsubstituted or substituted heterocyclyl.

The present disclosure also provides a compound of Formula (III):

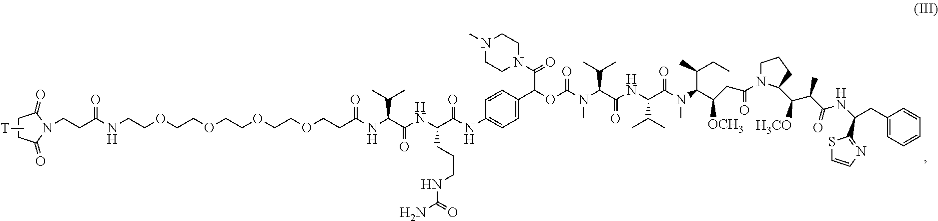

or a salt or solvate or stereoisomer thereof;
wherein T is a targeting moiety.

In some embodiments, provided is a compound of Formula (Ma):

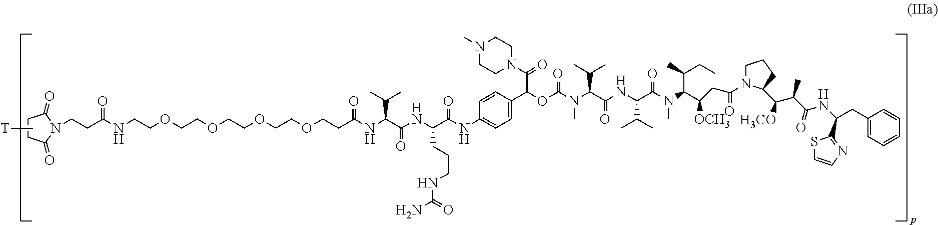

or a salt or solvate or stereoisomer thereof; wherein T is a targeting moiety and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure provides a compound of Formula (IV):

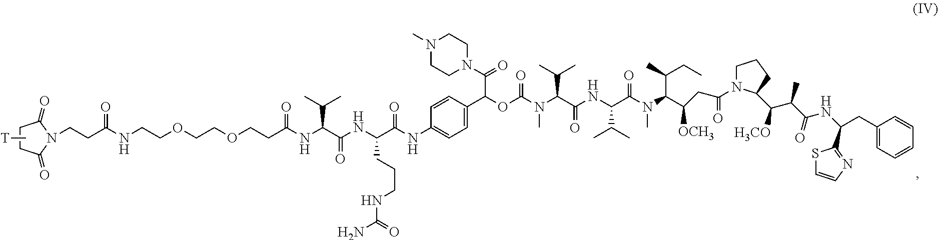

or a salt or solvate or stereoisomer thereof;
wherein T is a targeting moiety.

In some embodiments, provided is a compound of Formula (IVa):

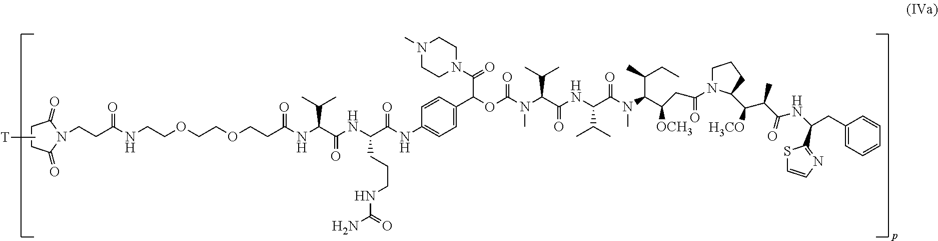

or a salt or solvate or stereoisomer thereof; wherein T is a targeting moiety and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure provides a compound of Formula (V):

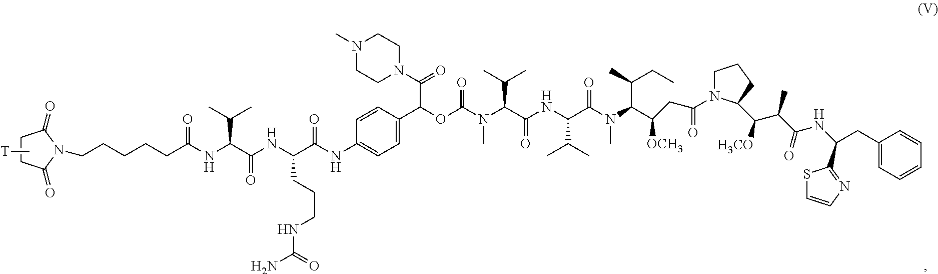

or a salt or solvate or stereoisomer thereof;
wherein T is a targeting moiety.

In some embodiments, provided is a compound of Formula (Va):

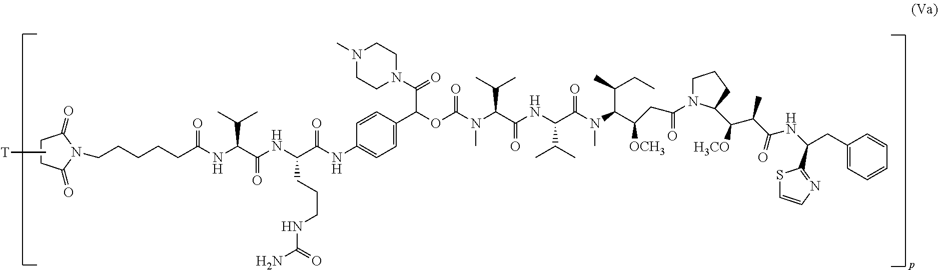

or a salt or solvate or stereoisomer thereof; wherein T is a targeting moiety and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure provides a compound of Formula (VI):

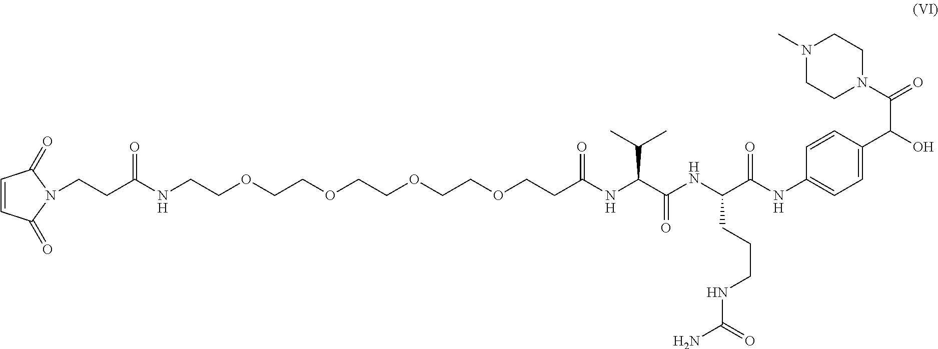 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N2CCN(C)CC2)cc1]

or a salt or solvate thereof.

The present disclosure provides a compound of Formula (VII):

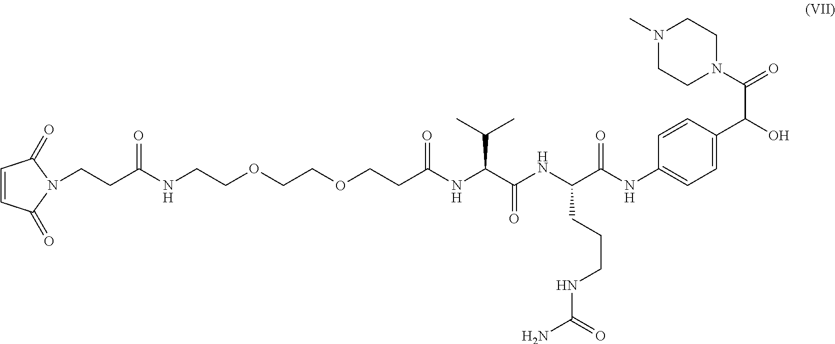 [SMILES: CC(C)[C@H](NC(=O)CCOCCOCCNC(=O)CCN1C(=O)C=CC1=O)C(=O)N[C@@H](CCCNC(N)=O)C(=O)Nc1ccc(C(O)C(=O)N2CCN(C)CC2)cc1]

or a salt or solvate thereof.

The present disclosure provides a compound of Formula (VIII):

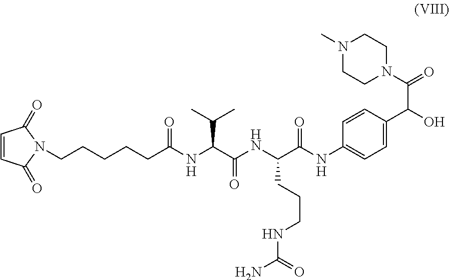 [SMILES: CC(C)[C@H](NC(=O)CCCCCN1C(=O)C=CC1=O)C(=O)N[C@@H](CCCNC(N)=O)C(=O)Nc1ccc(C(O)C(=O)N2CCN(C)CC2)cc1]

The present disclosure provides a compound of Formula (IX):

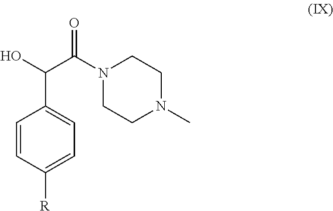

or a salt or solvate or stereoisomer thereof; wherein R is NO₂ or NH₂.

In certain embodiments, the compound of Formulae (I), (II), (III), (IV), (V), (VI), (VII), (VIII) or (IX) is a compound selected from those species described or exemplified in the detailed description herein.

In certain embodiments of the compound of Formulae (I), (II), (III), (IV), or (V) or (Ia), (IIa), (IIIa), (IVa), or (Va), T is an antibody targeting molecule. In some other embodiments, T is an anti-HER2 antibody. In further embodiments, the anti-HER2 antibody is humanized or monoclonal or a humanized monoclonal antibody. In some embodiments, T is the humanized monoclonal anti-HER2 antibody trastuzumab. In some embodiments the monoclonal anti-HER2 antibody is pertuzumab. In some embodiments, the monoclonal anti-HER2 antibody is margetuximab.

In further embodiments, one or more amino acid residues of the heavy chain and/or the light chain of the antibody are replaced with cysteine residues (e.g., engineered to comprise cysteine residue at a position not present in the parent antibody). In some embodiments, one or more amino acid residues of the Fc region of the antibody are replaced with a cysteine residue. In some embodiments, one or more amino acid residues of the antibody are at position 147, 188, 200, 201 and/or 206 of the light chain, and/or at position 155, 157, 165, 169, 197, 199, 209, 211 and/or 442 of the heavy chain using EU numbering (EU index in Kabat). In some embodiments, the antibody containing engineered cysteine residue is an anti-HER2 antibody. In some embodiments of the compound of Formulae (I), (II), (III), (IV), or (V) or (Ia), (IIa), (IIIa), (IVa), or (Va), D is linked to T by way of the cysteine (e.g. engineered) residue.

In certain embodiments, D is an amino-containing drug moiety. In some embodiments, D is connected to L¹ or X through the amino group. In further embodiments, D is duocarmycin, dolastatin, tubulysin, doxorubicin (DOX), paclitaxel, or mitomycin C (MMC), or an amino derivative thereof.

In a further aspect, the present disclosure provides a pharmaceutical composition comprising at least one compound of Formulae (I), (II), (III), (IV), or (V) or (Ia), (IIa), (IIIa), (IVa), or (Va) or a pharmaceutically acceptable salt thereof. Pharmaceutical compositions according to the embodiments may further comprise a pharmaceutically acceptable excipient.

The present disclosure also provides a compound of Formulae (I), (II), (III), (IV), or (V) or (Ia), (IIa), (IIIa), (IVa), or (Va) or a pharmaceutically acceptable salt thereof for use as a medicament.

In another aspect, the present disclosure provides a method of killing a cell, comprising administering to the cell an amount of the compound of Formulae (I), (II), (III), (IV), or (V) or (Ia), (IIa), (Ma), (IVa), or (Va) sufficient to kill the cell. In some embodiments, the cell is a cancer cell. In further embodiments, the cancer cell is a breast cancer cell, gastric cancer cell or ovarian cancer cell.

In another aspect, the present disclosure provides a method of treating cancer in an individual in need thereof comprising administering to the individual an effective amount of a compound of Formulae (I)-(V) or (Ia)-(Va) or a salt, a solvate, or a stereoisomer thereof. Examples of cancers that may be treated with the method described herein include, but are not limited to, carcinomas of the breast, bladder, pancreas, non-small-cell ling cancer (NSCLC), ovary, endometrium, colon, kidney, head and neck, stomach, esophagus, prostate, and testicular germ cell, uterine cancer, Wilm's tumor. In some embodiments, in the compound of Formulae (I), (II), (III), (IV), or (V) or (Ia), (IIa), (Ma), (IVa), or (Va), T is an anti-HER2 antibody and D is amino-containing drug moiety. In further embodiments, T is the antibody trastuzumab and D is monomethyl Dolastatin 10. In some embodiments, T is the antibody pertuzumab and D is monomethyl Dolastatin 10. In some embodiments, T is the antibody margetuximab and D is monomethyl Dolastatin 10.

In another aspect, the present disclosure provides a compound of Formula (I):

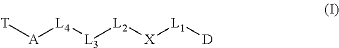

or a salt or solvate or stereoisomer thereof;
wherein:
D is a drug moiety;
T is a targeting moiety;
X is a hydrophilic self-immolative linker;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit,
for use in the treatment of cancer.

In another aspect, the present disclosure provides a compound of Formula (II):

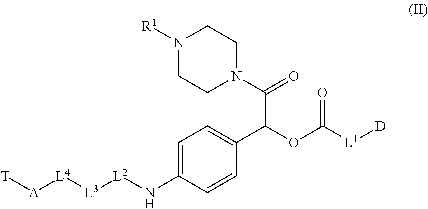

or a salt or solvate or stereoisomer thereof;
wherein:
D is a drug moiety;
T is a targeting moiety;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond, a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit,
for use in the treatment of cancer.

In another aspect, the present disclosure provides a compound of Formula (II):

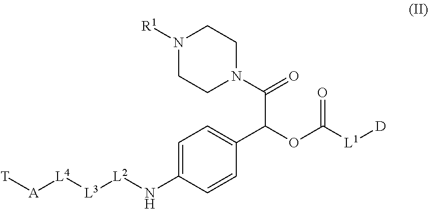

or a salt or solvate or stereoisomer thereof;
wherein:
D is a drug moiety;
T is a targeting moiety;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond, a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit,
for use in the treatment of cancer.

In another aspect, the present disclosure provides a compound of Formula (Ia):

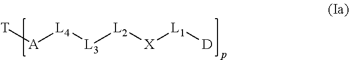

or a salt or solvate or stereoisomer thereof; wherein D, T, X, L¹, L², L³, L⁴ and A are as defined for Formula (I), and p is 1 to 20, for use in the treatment of cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In some embodiments, a compound of Formula (IIa) is provided:

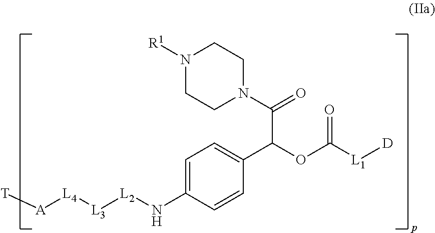

or a salt or solvate or stereoisomer thereof; wherein D, T, L¹, L², L³, L⁴ and A are as defined for Formula (II), and p is 1 to 20 for use in the treatment of cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In some aspects, the present disclosure also provides a compound of Formula III):

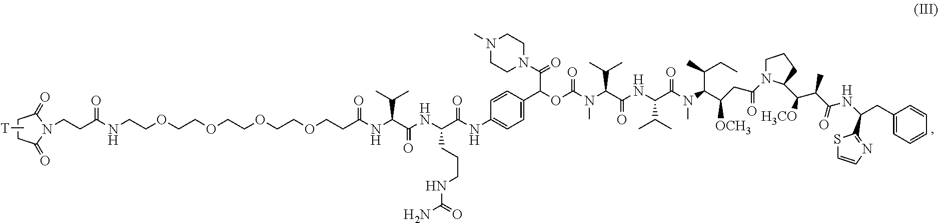

or a salt or solvate or stereoisomer thereof;
wherein T is a targeting moiety, for use in the treatment of cancer.

In some embodiments, provided is a compound of Formula (IIIa):

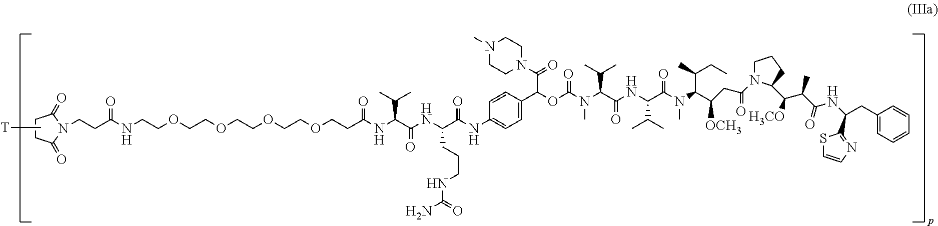

or a salt or solvate or stereoisomer thereof; wherein T is a targeting moiety and p is 1 to 20 for use in the treatment of cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure provides a compound of Formula (IV):

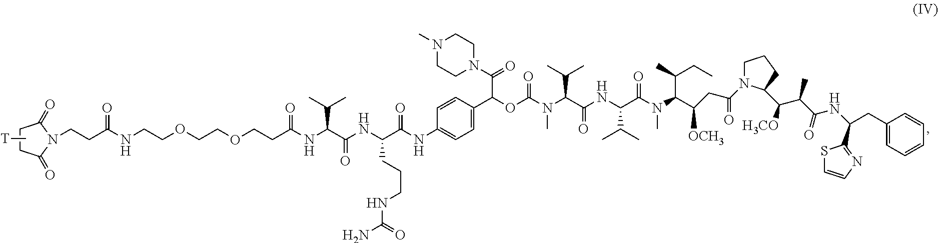

or a salt or solvate or stereoisomer thereof;
wherein T is a targeting moiety for use in the treatment of cancer.

In some embodiments, provided is a compound of Formula (IVa):

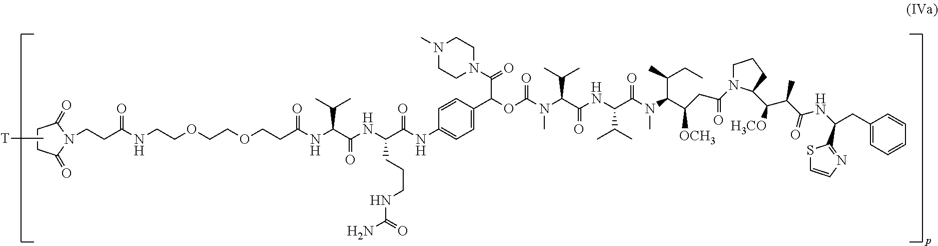

or a salt or solvate or stereoisomer thereof; wherein T is a targeting moiety and p is 1 to 20 for use in the treatment of cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure provides a compound of Formula (V):

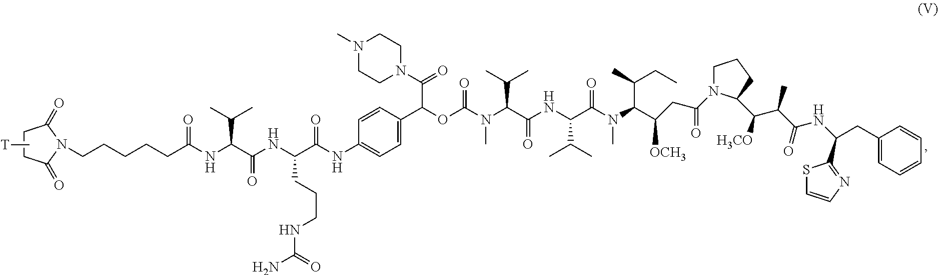

or a salt or solvate or stereoisomer thereof;
wherein T is a targeting moiety for use in the treatment of cancer.

In some embodiments, provided is a compound of Formula (Va):

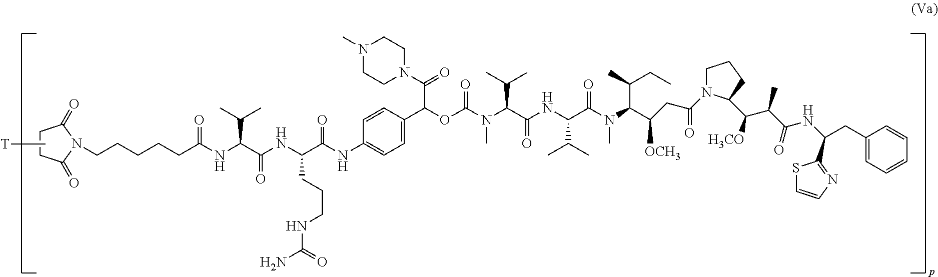

or a salt or solvate or stereoisomer thereof; wherein T is a targeting moiety and p is 1 to 20 for use in the treatment of cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In a further aspect, the present disclosure provides a pharmaceutical composition comprising at least one compound of Formulae (I), (II), (III), (IV), or (V) or (Ia), (IIa), (IIIa), (IVa), or (Va) or a pharmaceutically acceptable salt thereof for use in the treatment of cancer. Pharmaceutical compositions according to the embodiments may further comprise a pharmaceutically acceptable excipient. The present disclosure also provides a compound of Formulae (I), (II), (III), (IV), or (V) or (Ia), (IIa), (Ma), (IVa), or (Va) or a pharmaceutically acceptable salt thereof for use as a medicament.

In another aspect, the present disclosure provides the use of a compound of Formula (I):

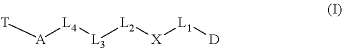

or a salt or solvate or stereoisomer thereof;
wherein:
D is a drug moiety;
T is a targeting moiety;
X is a hydrophilic self-immolative linker;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit,
in the manufacture of a medicament for treating cancer.

In another aspect, the present disclosure provides the use of a compound of Formula (II):

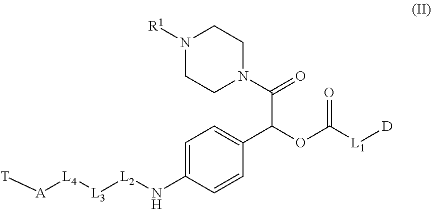

or a salt or solvate or stereoisomer thereof;
wherein:
D is a drug moiety;
T is a targeting moiety;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond, a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit,
in the manufacture of a medicament for treating cancer.

In another aspect, the present disclosure provides the use of a compound of Formula (II):

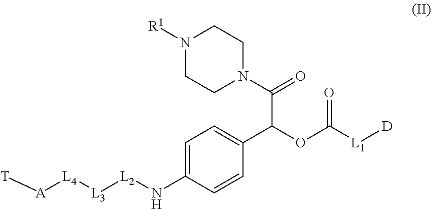

or a salt or solvate or stereoisomer thereof;
wherein:
D is a drug moiety;
T is a targeting moiety;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond, a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit,
in the manufacture of a medicament for treating cancer.

In another aspect, the present disclosure provides the use of a compound of Formula (Ia):

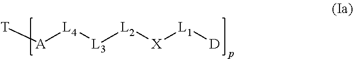

or a salt or solvate or stereoisomer thereof; wherein D, T, X, L¹, L², L³, L⁴ and A are as defined for Formula (I), and p is 1 to 20, in the manufacture of a medicament for treating cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In some embodiments, the use of a compound of Formula (IIa) is provided:

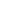

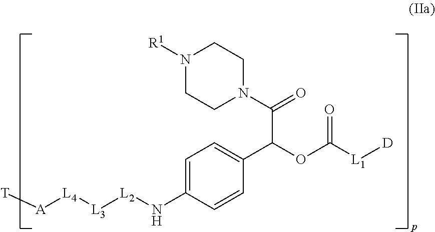

or a salt or solvate or stereoisomer thereof; wherein D, T, L¹, L², L³, L⁴ and A are as defined for Formula (II), and p is 1 to 20 in the manufacture of a medicament for treating cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In some aspects, the present disclosure also provides the use of a compound of Formula (III):

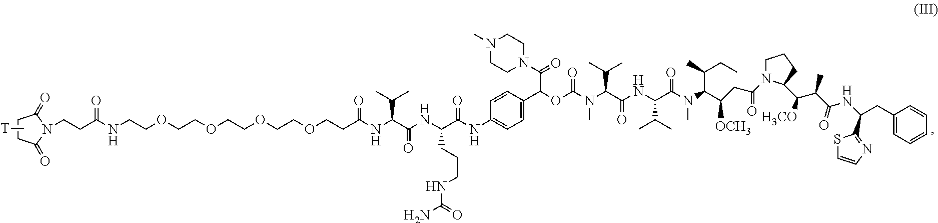

or a salt or solvate or stereoisomer thereof;
wherein T is a targeting moiety, in the manufacture of a medicament for treating cancer.

In some embodiments, provided is the use of a compound of Formula (Ma):

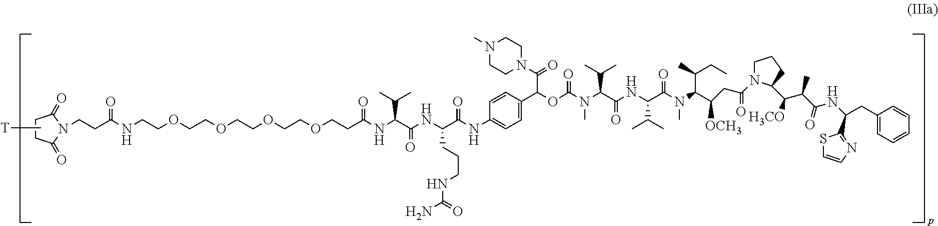

or a salt or solvate or stereoisomer thereof; wherein T is a targeting moiety and p is 1 to 20 in the manufacture of a medicament for treating cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure provides the use of a compound of Formula (IV):

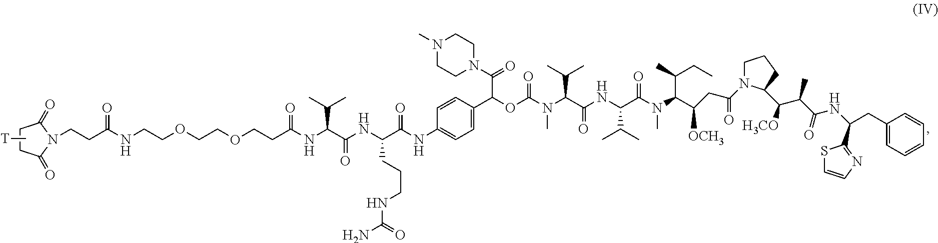

or a salt or solvate or stereoisomer thereof;
wherein T is a targeting moiety in the manufacture of a medicament for treating cancer.

In some embodiments, provided is the use of a compound of Formula (IVa):

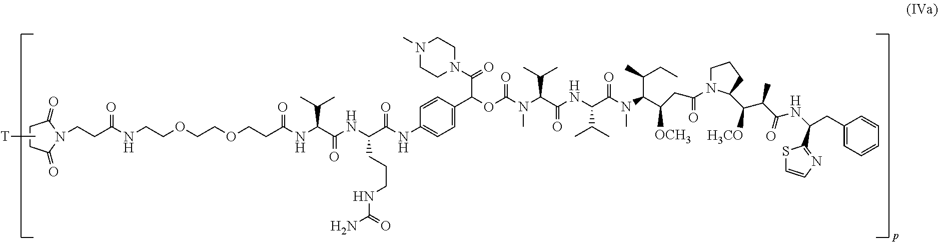

or a salt or solvate or stereoisomer thereof; wherein T is a targeting moiety and p is 1 to 20 in the manufacture of a medicament for treating cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure provides the use of a compound of Formula (V):

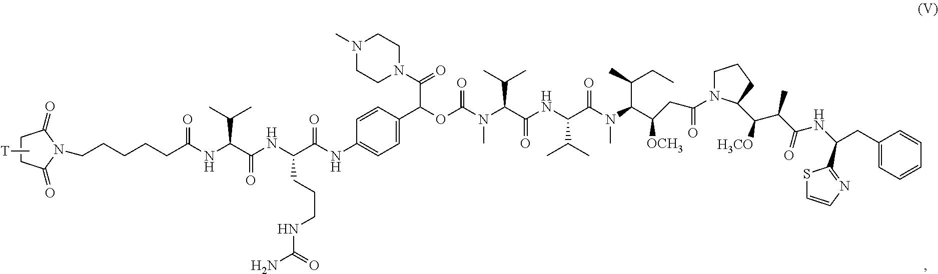

or a salt or solvate or stereoisomer thereof;
wherein T is a targeting moiety in the manufacture of a medicament for treating cancer.

In some embodiments, provided is the use of a compound of Formula (Va):

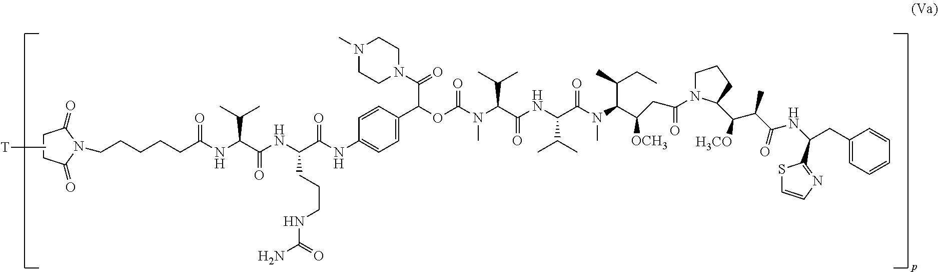

or a salt or solvate or stereoisomer thereof; wherein T is a targeting moiety and p is 1 to 20 in the manufacture of a medicament for treating cancer. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

In a some aspects, the present disclosure provides the use of a pharmaceutical composition comprising at least one compound of Formulae (I), (II), (III), (IV), or (V) or (Ia), (IIa), (Ma), (IVa), or (Va) or a pharmaceutically acceptable salt thereof in the manufacture of a medicament for treating cancer. Pharmaceutical compositions according to the embodiments may further comprise a pharmaceutically acceptable excipient. The present disclosure also provides a compound of Formulae (I), (II), (III), (IV), or (V) or (Ia), (IIa), (IIIa), (IVa), or (Va) or a pharmaceutically acceptable salt thereof for use as a medicament.

In any of the embodiments discussed above, the anti-HER2 antibody comprises a heavy chain variable region and a light chain variable region, wherein

(1) the heavy chain variable region comprises the three heavy chain CDRs of the amino acid sequence of SEQ ID NO:16-18 and/or the light chain variable region comprises the three light chain CDRs of the amino acid sequence of SEQ ID NO:19-21;
(2) the heavy chain variable region comprises the three heavy chain CDRs of the amino acid sequence of SEQ ID NO:22-24 and/or the light chain variable region comprises the three light chain CDRs of the amino acid sequence of SEQ ID NO:25-27; or
(3) the heavy chain variable region comprises the three heavy chain CDRs of the amino acid sequence of SEQ ID NO:28-30 and/or the light chain variable region comprises the three light chain CDRs of the amino acid sequence of SEQ ID NO:31-33.

In any of the embodiments discussed above, the anti-HER2 antibody comprises a heavy chain variable region and a light chain variable region, wherein

(1) the heavy chain variable region comprises the amino acid sequence of SEQ ID NO:8 and/or the light chain variable region comprises the amino acid sequence of SEQ ID NO:7;
(2) the heavy chain variable region comprises the amino acid sequence of SEQ ID NO:13 and/or the light chain variable region comprises the amino acid sequence of SEQ ID NO:12;
(3) the heavy chain variable region comprises the amino acid sequence of SEQ ID NO:15 and/or the light chain variable region comprises the amino acid sequence of SEQ ID NO:14. Additional embodiments, features, and advantages of the disclosure will be apparent from the following detailed description and through practice of the disclosure.

### BRIEF DESCRIPTION OF THE DRAWINGS

FIG. 1 shows an NMR spectrum of Tap-18H.

FIG. 2 shows an NMR spectrum of Tap-18Hr1.

FIG. 3 shows an NMR spectrum of Tap-18Hr2.

FIG. 4 shows the in vivo anti-tumor activity of anti-HER2-IgG1/TAP18Hr1 against ovarian cancer SKOV-3.

FIG. 5 shows the in vivo anti-tumor activity of Anti-HER2-IgG1/TAP18Hr1 against breast cancer MDA-MB-453. Arrow indicates the time of ADC treatment (day 1).

FIG. 6 shows the in vivo anti-tumor activity of Anti-HER2-IgG1/TAP18Hr1 against gastric cancer NCI-N87. Arrow indicates the time of ADC treatment (day 1 and day 22).

FIG. 7 shows the in vivo anti-tumor activity of site-specific conjugated Anti-HER2-Cys variants against gastric cancer NCI-N87. Arrow indicates the time of ADC treatment (day 1).

FIG. 8 shows the in vivo anti-tumor activity of Tap18Hr1 conventional conjugated Anti-HER2 and site-specific conjugated Anti-HER2-Cys variants against breast cancer JIMT-1. Arrow indicated the time of ADC treatment (day1).

### DETAILED DESCRIPTION

The present disclosure provides compounds with a hydrophilic self-immolative linker, which may be cleavable under appropriate conditions and incorporates a hydrophilic group to provide better solubility of the compound. The hydrophilic self immolative linker may provide increased solubility of drug conjugates for cytotoxic drugs which are often hydrophobic. Other advantages of using a hydrophilic self-immolative linker in a drug conjugate include increased stability of the drug conjugate and decreased aggregation of the drug conjugate.

The present disclosure provides drug conjugates that may have superior serum stability. For example, in contrast to drug conjugates wherein a hydroxyl group of a drug is linked to a spacer via a labile carbonate linkage that is susceptible to rapid hydrolysis in aqueous buffer or human serum, the drug conjugates of the present application utilize a benzyloxycarbonyl linkage. These conjugates are relatively more stable under the same conditions, and selectively undergo fragmentation to release the drug upon treatment with protease, e.g., cathepsin B. Serum stability is a desirable property for drug conjugates where it is desired to administer inactive drug to the patient's serum, have that inactive drug concentrate at a target by way of the ligand, and then have that drug conjugate converted to an active form only in the vicinity of the target.

The present disclosure provides drug conjugates which may have decreased aggregation. Increased associated hydrophobicity of some enzyme-labile linkers may lead to aggregation of drug conjugates, particularly with strongly hydrophobic drugs. With incorporation of a hydrophilic group into the linker, there is decreased aggregation of the drug conjugate. Compared to ADC with chemically-labile linker and noncleavable linkers, the linkers described herein can achieve better serum stability via specific enzyme-labile design, as well as achieve better efficacy via bystander effect on the heterogeneous cancer cells. The compounds of the present disclosure comprise a drug moiety, a targeting moiety capable of targeting a selected cell population, and a linker which contains an acyl unit, an optional spacer unit for providing distance between the drug moiety and the targeting moiety, a peptide linker which can be cleavable under appropriate conditions, a hydrophilic self-immolative linker, and an optional second self-immolative spacer or cyclization self-elimination linker. Each of the features is discussed below.

“Alkyl” refers to monovalent saturated aliphatic hydrocarbyl groups having 1, 2, 3, 4, 5, 6, 7, 8, 9, or 10 carbon atoms and preferably 1, 2, 3, 4, 5, or 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), neopentyl ((CH₃)₃CCH₂—), and n-hexyl (CH₃(CH₂)₅—).

“Alkylene” refers to divalent aliphatic hydrocarbylene groups preferably having 1, 2, 3, 4, 5, 6, 7, 8, 9, or 10 and more preferably 1, 2, or 3 carbon atoms that are either straight-chained or branched. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Alkenyl” refers to straight chain or branched hydrocarbyl groups having 2, 3, 4, 5, 6, 7, 8, 9, or 10 carbon atoms and preferably 2, 3, or 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

“Alkenylene” refers to straight chain or branched hydrocarbylene groups having 2, 3, 4, 5, 6, 7, 8, 9, or 10 carbon atoms and preferably 2, 3, or 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

“Alkynyl” refers to straight or branched hydrocarbyl groups having 2, 3, 4, 5, or 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Alkynylene” refers to straight or branched hydrocarbylene groups having from 2, 3, 4, 5, or 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Amino” refers to the group —NH₂.

“Substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aryl” refers to a monovalent aromatic carbocyclic group of 6, 7, 8, 9, 10, 11, 12, 13, 14, 15, 16, 17, or 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with 1, 2, 3, 4, or 5 substituents, or from 1, 2, or 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Cycloalkyl” refers to cyclic alkyl groups of 3, 4, 5, 6, 7, 8, 9, or 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

“Heteroaryl” refers to an aromatic group of 1, 2, 3, 4, 5, 6, 7, 8, 9, 10, 11, 12, 13, 14, or 15 carbon atoms, such as 1, 2, 3, 4, 5, 6, 7, 8, 9, or 10 carbon atoms and 1, 2, 3, 4, 5, 6, 7, 8, 9, or 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1, 2, 3, 4, or 5 substituents, or from 1, 2, or 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

Examples of heteroaryls include, but are not limited to, pyrrole, imidazole, pyrazole, pyridine, pyrazine, pyrimidine, pyridazine, indolizine, isoindole, indole, purine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, piperidine, piperazine, phthalimide, 4,5,6,7-tetrahydrobenzo[b]thiophene, thiazole, thiophene, benzo[b]thiophene, and the like.

“Heterocycle,” “heterocyclic,” “heterocycloalkyl” or “heterocyclyl” refers to a saturated or partially unsaturated group having a single ring or multiple condensed rings, including fused, bridged, or Spiro ring systems, and having 3, 4, 5, 6, 7, 8, 9, 10, 11, 12, 13, 14, 15, 16, 17, 18, 19, or 20 ring atoms, including 1, 2, 3, 4, 5, 6, 7, 8, 9, or 10 hetero atoms. These ring atoms are selected from the group consisting of carbon, nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for N-oxide, —S(O)—, or —SO₂— moieties.

Examples of heterocycles include, but are not limited to, azetidine, dihydroindole, indazole, quinolizine, imidazolidine, imidazoline, piperidine, piperazine, indoline, 1,2,3,4-tetrahydroisoquinoline, thiazolidine, morpholinyl, thiomorpholinyl (also referred to as thiamorpholinyl), 1,1-dioxothiomorpholinyl, piperidinyl, pyrrolidine, tetrahydrofuranyl, and the like.

Where a heteroaryl or heterocyclyl group is “substituted,” unless otherwise constrained by the definition for the heteroaryl or heterocyclic substituent, such heteroaryl or heterocyclic groups can be substituted with 1, 2, 3, 4, or 5, or from 1, 2, or 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, azido, cyano, halogen, hydroxyl, oxo, thioketo, carboxyl, carboxyl ester, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, sulfonylamino, —SO-alkyl, —SO-substituted alkyl, —SO-aryl, —SO-heteroaryl, —SO-heterocyclyl, —SO₂-alkyl, —SO₂-substituted alkyl, —SO₂-aryl, —SO₂-heteroaryl, and —SO₂-heterocyclyl.

“Polyalkylene glycol” refers to straight or branched polyalkylene glycol polymers such as polyethylene glycol, polypropylene glycol, and polybutylene glycol. A polyalkylene glycol subunit is a single polyalkylene glycol unit. For example, an example of a polyethylene glycol subunit would be an ethylene glycol, —O—CH₂—CH₂—O—, or propylene glycol, —O—CH₂—CH₂—CH₂—O—, capped with a hydrogen at the chain termination point. Other examples of poly(alkylene glycol) include, but are not limited to, PEG, PEG derivatives such as methoxypoly(ethylene glycol) (mPEG), poly(ethylene oxide), PPG, poly(tetramethylene glycol), poly(ethylene oxide-co-propylene oxide), or copolymers and combinations thereof.

“Polyamine” refers to polymers having an amine functionality in the monomer unit, either incorporated into the backbone, as in polyalkyleneimines, or in a pendant group as in polyvinyl amines.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, SR⁷⁰, —NR⁸⁰R⁸⁰, trihalomethyl, —CN, —OCN, —SCN, —NO, —NO₂, ═N₂, —N₃, —S(O)R⁷⁰, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heterocycloalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 3-, 4-, 5-, 6-, or 7-membered heterocycloalkyl which may optionally include from 1 to 4 of the same or different additional heteroatoms selected from the group consisting of O, N and S, of which N may have —H, C₁-C₄ alkyl, —C(O)C₁₋₄alkyl, —CO₂C₁₋₄alkyl, or —SO₂C₁₋₄alkyl substitution; and each M⁺ is a counter ion with a net single positive charge. Each M may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the embodiments and the other a typical counter ion such as chloride, or two ionized compounds disclosed herein can serve as counter ions for such divalent alkali earth ions, or a doubly ionized compound of the embodiments can serve as the counter ion for such divalent alkali earth ions).

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —S(O)R⁷⁰, —SO₂R⁷⁰, —SO₃⁻M⁺, —SO₃R⁷⁰, —OSO₂R⁷⁰, —OSO₃M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰—, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰ where R⁶⁰, R⁷⁰, R⁸⁰ and M⁺are as previously defined, provided that in case of substituted alkene or alkyne, the substituents are not —O⁻M⁺, —OR⁷⁰, —SR⁷⁰, or —S⁻M⁺. In addition to the substituent groups disclosed with respect to the individual terms herein, substituent groups for hydrogens on nitrogen atoms in “substituted” heterocycloalkyl and cycloalkyl groups are, unless otherwise specified, —R⁶⁰, —O⁻M⁺, —OR⁷⁰, —SR⁷⁰, —S³¹ M⁺, —NR⁸⁰R⁸⁰ trihalomethyl, —CF₃, —CN, —NO, —NO₂, —S(O)R⁷⁰, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R)⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC (S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰, and —NR⁷⁰C(NR⁷⁰)NR⁷⁰R⁷⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent. It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds. The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

A wavy line in the structure drawing of a group represents an attachment point of the group to the parent structure.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible. It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

As used herein, an “effective dosage” or “effective amount” of drug, compound, conjugate, drug conjugate, antibody drug conjugate, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of the present disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “administration” refers to a generic term for the dispensing of a therapeutic agent to treat a condition. In some embodiments, the pharmaceutical compositions of the present disclosure contain a pharmaceutically acceptable carrier or excipient suitable for rendering the compound or mixture administrable orally as a tablet, capsule or pill, or rendering the compound suitable for parenteral, intravenous, intradermal, intramuscular, intraperitoneal, intranasal, sublingual, intratracheal, inhalation, ocular, vaginal, rectal, subcutaneous, or transdermal administration.

A variety of administration routes are available. The particular mode selected will depend, of course, upon the particular agent or agents selected, the particular condition being treated, and the dosage required for therapeutic efficacy. Several modes of administration are discussed below.

Administering the compounds or pharmaceutical compositions of the present disclosure may be accomplished by any means known to the skilled artisan. Routes of administration include but are not limited to oral, parenteral, intravenous, intramuscular, intraperitoneal, intranasal, sublingual, intratracheal, inhalation, subcutaneous, ocular, vaginal, and rectal. Systemic routes include oral and parenteral.

For oral administration, the compounds can be formulated readily by combining the active compound(s) with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Optionally the oral formulations may also be formulated in saline or buffers for neutralizing internal acid conditions or may be administered without any carriers.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

As used herein, “in conjunction with” refers to administration of one treatment modality in addition to another treatment modality. As such, “in conjunction with” refers to administration of one treatment modality before, during or after administration of the other treatment modality to the individual.

As used herein, “treatment” or “treating” is an approach for obtaining beneficial or desired results including and preferably clinical results. For purposes of the present disclosure, beneficial or desired clinical results include, but are not limited to, one or more of the following: reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “individual” or a “subject” is a mammal, more preferably a human. Mammals also include, but are not limited to, farm animals, sport animals, pets (such as cats, dogs, horses), primates, mice and rats. “Treatment of cancer in an individual in need thereof” is an individual identified as having cancer, i.e. the individual has been diagnosed by a physician (e.g. using methods well known in the art) as having cancer. In some embodiments, the individual in need of treatment is an individual suspected of having or developing cancer. Examples of individuals suspected of having or developing cancer include but are not limited to subjects identified as having mutations associated with cancer or the development of cancer, subjects with a family history of cancer, and subjects who have previously had or been cured of cancer (including cancer patients in remission).

As used herein, the term “specifically recognizes” or “specifically binds” refers to measurable and reproducible interactions such as attraction or binding between a target and an antibody (or a molecule or a moiety), that is determinative of the presence of the target in the presence of a heterogeneous population of molecules including biological molecules. For example, an antibody that specifically or preferentially binds to an epitope is an antibody that binds this epitope with greater affinity, avidity, more readily, and/or with greater duration than it binds to other epitopes of the target or non-target epitopes. It is also understood that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, the terms “cancer,” “tumor,” “cancerous,” and “malignant” refer to or describe the physiological condition in mammals that is typically characterized by unregulated cell growth. Examples of cancer include but are not limited to, carcinoma, including adenocarcinoma, lymphoma, blastoma, melanoma, and sarcoma. More particular examples of such cancers include squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, glioma, ovarian cancer, liver cancer such as hepatic carcinoma and hepatoma, bladder cancer, breast cancer, colon cancer, colorectal cancer, endometrial or uterine carcinoma, salivary gland carcinoma, kidney cancer such as renal cell carcinoma and Wilms' tumors, basal cell carcinoma, melanoma, mesothelioma, prostate cancer, thyroid cancer, testicular cancer, esophageal cancer, gallbladder cancer, and various types of head and neck cancer.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

Reference to “about” a value or parameter herein includes (and describes) embodiments that are directed to that value or parameter per se. For example, description referring to “about X” includes description of “X.”

It is understood that aspect and variations of the disclosure described herein include “consisting” and/or “consisting essentially of” aspects and variations.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 4^{th} edition, New York: Oxford University Press, 2002, pp. 360-361, 1084-1085; Smith and March, March's Advanced Organic Chemistry: Reactions, Mechanisms, and Structure, 5^{th} edition, Wiley-Interscience, 2001. The nomenclature used herein to name the subject compounds is illustrated in the Examples herein. This nomenclature has generally been derived using the commercially-available AutoNom software (MDL, San Leandro, Calif.).

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

The present disclosure provides a compound of Formula (I):

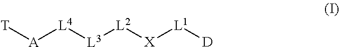

or a salt or solvate or stereoisomer thereof;
wherein:
D is a drug moiety;
T is a targeting moiety;
X is a hydrophilic self-immolative linker;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit.

The present disclosure also provides a compound of Formula (II):

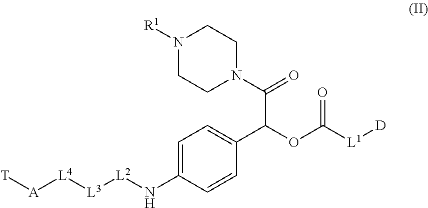

or a salt or solvate or stereoisomer thereof;
wherein:
D is a drug moiety;
T is a targeting moiety;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond, a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit.

In some embodiments, the targeting moiety has one or more attachment sites for linking to the drug moiety. For example, a targeting moiety T can have multiple sites for linking to a linker-drug moiety (e.g., A-L⁴-L³-L²-X-L¹-D). Thus, also provided is a compound of Formula (Ia):

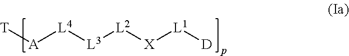

or a salt or solvate or stereoisomer thereof, wherein X, L¹, L², L³, L⁴ and A are as defined for Formula (I), and p is 1 to 20. In some embodiments, p is 1 to 8. In some embodiments, p is 1 to 6. In some embodiments, p is 1 to 4. In some embodiments, p is 2 to 4. In some embodiments,
p is 1, 2, 3, 4, 5, 6, 7, 8, 9, 10, 11, 12, 13, 14, 15, 16, 17, 18, 19, or 20. In some embodiments, p is 1, 2, 3, or 4.

The present disclosure also provides a compound of Formula (IIa):

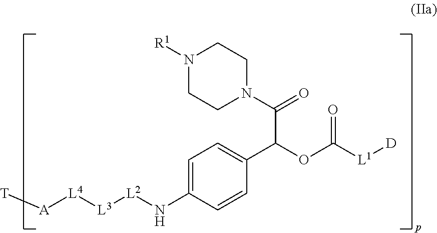

or a salt or solvate or stereoisomer thereof, wherein R¹, X, L¹, L², L³, L⁴ and A are as defined for Formula (II), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4.

### Peptide Linker

In some embodiments, L³ is a peptide linker. In some embodiments, L³ is a peptide linker of 1, 2, 3, 4, 5, 6, 7, 8, 9, or 10 amino acid residues. In certain embodiments, L³ is a peptide linker of 2, 3, or 4 amino acid residues. In certain instances, L³ is a dipeptide linker. An amino acid residue can be a naturally-occurring or non-natural amino acid residue.

The terms “natural amino acid” and “naturally-occurring amino acid” refer to Ala, Asp, Cys, Glu, Phe, Gly, His, Ile, Lys, Leu, Met, Asn, Pro, Gln, Arg, Ser, Thr, Val, Trp, and Tyr. “Non-natural amino acids” (i.e., amino acids do not occur naturally) include, by way of non-limiting example, homoserine, homoarginine, citrulline, phenylglycine, taurine, iodotyrosine, seleno-cysteine, norleucine (“Nle”), norvaline (“Nva”), beta-alanine, L- or D-naphthalanine, ornithine (“Orn”), and the like.

Amino acids also include the D-forms of natural and non-natural amino acids. “D-” designates an amino acid having the “D” (dextrorotary) configuration, as opposed to the configuration in the naturally occurring (“L-”) amino acids. Where no specific configuration is indicated, one skilled in the art would understand the amino acid to be an L-amino acid. The amino acids can, however, also be in racemic mixtures of the D- and L-configuration. Natural and non-natural amino acids can be purchased commercially (Sigma Chemical Co.; Advanced Chemtech) or synthesized using methods known in the art. Amino acid substitutions may be made on the basis of similarity in polarity, charge, solubility, hydrophobicity, hydrophilicity, and/or the amphipathic nature of the residues as long as their biological activity is retained.

The amino acid residue sequence can be specifically tailored so that it will be selectively enzymatically cleaved from the resulting peptidyl derivative drug-conjugate by one or more of the tumor-associated proteases.

In certain embodiments, L³ is a peptide linker comprising at least one lysine or at least one arginine residue.

In certain embodiments, L³ is a peptide linker comprising an amino acid residue selected from lysine, D-lysine, citrulline, arginine, proline, histidine, ornithine and glutamine.

In certain embodiments, L³ is a peptide linker comprising an amino acid residue selected from valine, isoleucine, phenylalanine, methionine, asparagine, proline, alanine, leucine, tryptophan, and tyrosine.

In certain embodiments, L³ is a dipeptide linker selected from valine-citrulline, proline-lysine, methionine-D-lysine, asparagine-D-lysine, isoleucine-proline, phenylalanine-lysine, and valine-lysine. In certain embodiments, L³ is valine-citrulline.

Numerous specific peptide linker molecules suitable for use in the present disclosure can be designed and optimized in their selectivity for enzymatic cleavage by a particular tumor-associated protease. Certain peptide linkers for use in the present disclosure are those which are optimized toward the proteases, cathepsin B and D.

### Hydrophilic Self-Immolative Linker

In some embodiments of the compounds described herein, X is a hydrophilic self-immolative linker.

The compound of the present disclosure employs a hydrophilic self-immolative spacer moiety which spaces and covalently links together the two functional moieties and incorporates a hydrophilic group, which provides better solubility of the compound. In some embodiments, the hydrophilic self-immolative spacer moiety links together a targeting moiety and a drug moiety. Increased associated hydrophobicity of some enzyme-labile linkers can lead to aggregation of drug conjugates, particularly with strongly hydrophobic drugs. With incorporation of a hydrophilic group into the linker, there will be a decreased aggregation of the drug conjugate.

A self-immolative spacer may be defined as a bifunctional chemical moiety which is capable of covalently linking together two spaced chemical moieties into a normally stable tripartite molecule, can release one of the spaced chemical moieties from the tripartite molecule by means of enzymatic cleavage; and following enzymatic cleavage, can spontaneously cleave from the remainder of the molecule to release the other of the spaced chemical moieties. In certain embodiments, X is a benzyloxycarbonyl group. In certain embodiments, X is

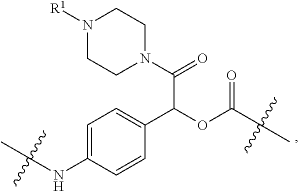

wherein R¹ is hydrogen, unsubstituted or substituted C₁₋₃ alkyl, or unsubstituted or substituted heterocyclyl.

In some embodiments, the present disclosure provides a compound of Formula (II):

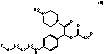

or a salt or solvate or stereoisomer thereof;
wherein:
D is a drug moiety;
T is a targeting moiety;
R¹ is hydrogen, unsubstituted or substituted C₁₋₃ alkyl, or unsubstituted or substituted heterocyclyl;
L¹ is a bond, a self-immolative linker, or a cyclization self-elimination linker;
L² is a bond, a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit.

Also provided is a compound of Formula (IIa):

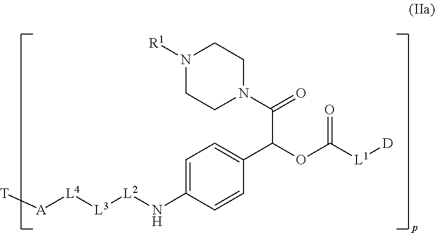

or a salt or solvate or stereoisomer thereof; wherein D, T, L¹, L², L³, L⁴ and A are as defined for Formula (II), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3, 4, 5, 6, 7, 8, 9, 10, 11, 12, 13, 14, 15, 16, 17, 18, 19, or 20. In some embodiments, p is 1, 2, 3, or 4. In some embodiments, p is 2. In some embodiments, p is 3. In some embodiments, p is 4.

In certain embodiments of Formula (II) or (IIa), R¹ is hydrogen. In certain instances, R¹ is methyl.

The release of the drug moiety is based on the self-elimination reaction of aminobenzyloxycarbonyl group. For illustration purposes, a reaction scheme with an aminobenzyloxycarbonyl group with a drug and peptide attached is shown below.

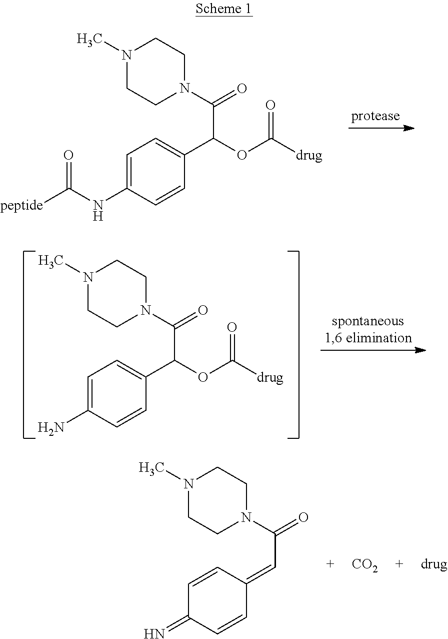

Referring to Scheme 1, upon cleavage from a peptide, an aminobenzyloxycarbonyl is formed and is able to undergo a spontaneous 1,6 elimination to form a cyclohexa-2,5-dienimine derivative and carbon dioxide and release the drug.

### Optional Second Self-Immolative Linker or Cyclization Self-Elimination Linker

An optional second self-immolative linker or cyclization self-elimination linker provides an additional linker for allowance of fine-tuning the cleavage of the compound to release the drug moiety.

In the compounds described herein, L¹ is a bond, a self-immolative linker, or a cyclization self-elimination linker; L² is a bond or a self-immolative linker; wherein if L¹ is a self-immolative linker or a cyclization self-elimination linker, then L² is a bond; and wherein if L² is a self-immolative linker, then L¹ is a bond. Thus, there is an optional second self-immolative linker or a cyclization self-elimination linker adjacent the hydrophilic self-immolative linker.

In certain embodiments, L¹ is a bond and L² is a bond. In certain embodiments, L¹ is a self-immolative linker or a cyclization self-elimination linker and L² is a bond. In certain embodiments, L¹ is a bond and L² is a self-immolative linker.

In some embodiments, L¹ is a bond. In certain embodiments, L¹ is a self-immolative spacer or a cyclization self-elimination linker, which separates the hydrophilic self-immolative linker and the drug moiety. In certain embodiments, L¹ is an aminobenzyloxycarbonyl linker. In certain embodiments, L¹ is selected from:

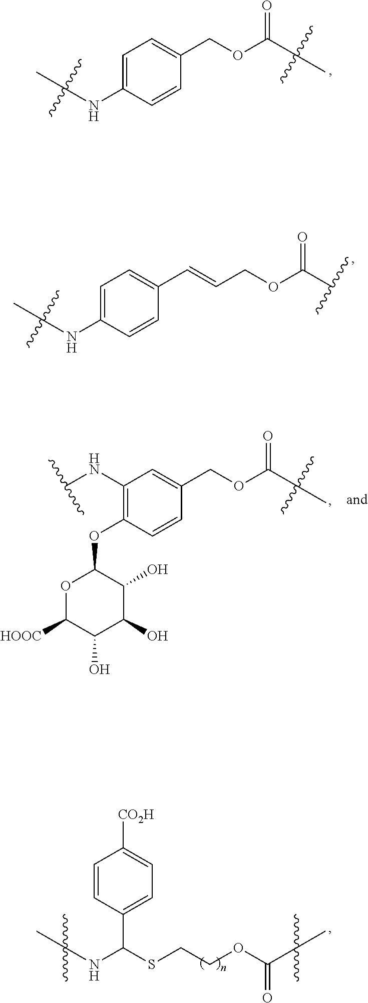

wherein n is 1 or 2.

In certain instances, the self-immolative linker or cyclization self-elimination linker provides design potential for a wider variety of moieties that can be used. For example, in Formula (IV) or (IVa), a carbamate linkage (—O—C(O)—N(H)—) linkage between the hydrophilic self-immolative linker and the drug moiety would provide a stable drug conjugate and would readily cleave to provide a free drug moiety. The hydrophilic self-immolative linker will typically terminate with an oxycarbonyl group (—O—C(O)—). If the drug moiety has an amino-reactive group that may be used to react to form a carbamate group, then the (optional) second self-immolative unit or cyclization self-elimination linker is not necessary; although it may still be employed. However, if the drug does not contain an amino group, but instead contains some other reactive functional group, then such drugs may still be incorporated into an aminobenzyloxycarbonyl-containing compound of the present embodiments by including an intermediate self-immolative spacer or cyclization self-elimination linker between the drug moiety and the aminobenzyloxycarbonyl group.

The cyclization self-elimination linkers of L¹ below provide linkage of hydroxyl-containing or thiol-containing drug moieties to the aminobenzyloxycarbonyl group of the hydrophilic self-immolative linker:

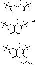

The cyclization self-elimination linkers in the compounds of the embodiments provide for cleavage of the compound to release the drug moiety. The elimination mechanism of the adjacent hydrophilic self-immolative linker would reveal an amino group of L¹. The amino group can then react with the carbamate group or thiocarbamate linkage of L¹ and the drug moiety in a cyclization reaction to release the hydroxyl-containing or thiol-containing drug moiety.

In some embodiments, L² is a bond. In certain embodiments, L² is a self-immolative spacer which separates the hydrophilic self-immolative linker and the peptide linker. In certain embodiments, L² is an aminobenzyloxycarbonyl linker.

In certain embodiments, L² is selected from

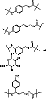

wherein n is 1 or 2.

### Optional Spacer

In some embodiments, L⁴ is a bond or a spacer. In certain embodiments, L⁴ is a bond. In certain embodiments, L⁴ is a spacer, which can provide distance between the drug moiety and the targeting moiety.

In certain embodiments, a spacer is selected from alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, and heteroatoms, and combinations thereof. The spacer can be homogenous or heterogeneous in its atom content (e.g., spacers containing only carbon atoms or spacers containing carbon atoms as well as one or more heteroatoms present on the spacer. Preferably, the spacer contains 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 carbon atoms and 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 heteroatoms selected from oxygen, nitrogen and sulfur. The spacer may also be chiral or achiral, linear, branched or cyclic.

In certain embodiments, L⁴ is a spacer selected from polyalkylene glycol, alkylene, alkenylene, alkynylene, and polyamine. Examples of alkenylene include, but is not limited to, vinylene (—CH═CH—), allylene (—CH₂C≡C—), and but-3-en-1-ylene (—CH₂ CH₂C≡CH—). Examples of alkenylene include, but is not limited to, acetylenylene (—C≡C—), and propargylene (—CH₂C≡C—).

In certain embodiments, L⁴ is a spacer that comprises a functional group that can provide linkage to the terminal end of the peptide linkage. Functional groups, such as C(O), C(O)—NH, S(O)₂, and S(O)₂—NH, can provide linkage to the terminal end of the peptide linkage. In certain instances, L⁴ is L^{4a}-C(O), L^{4a}-C(O)—NH, L^{4a}-S(O)₂, L^{4a}-S(O)₂—NH, wherein L^{4a} is selected from polyalkylene glycol, alkylene, alkenylene, alkynylene, and polyamine. In certain instances, L⁴ is L^{4a}-C(O), wherein L^{4a} is selected from polyalkylene glycol, alkylene, alkenylene, alkynylene, and polyamine.

In certain embodiments, L⁴ is L^{4a}-C(O), wherein L^{4a} is a polyalkylene glycol. In certain embodiments, L⁴ is L^{4a}-C(O), wherein L^{4a} is a polyethylene glycol. In certain embodiments, the spacer is of the formula —CH₂—(CH₂—O—CH₂)ₘ—CH₂—C(O)—, wherein m is the integer 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. In certain embodiments, L⁴ is L^{4a}-C(O), wherein L^{4a} is alkylene. In certain embodiments, L⁴ is L^{4a}-C(O), wherein L^{4a} is C₁₋₁₀alkylene, C₁₋₈alkylene, or C₁₋₆alkylene. In certain embodiments, L⁴ is L^{4a}-C(O), wherein L^{4a} is C₄alkylene, C₅alkylene, or C₆alkylene. In certain embodiments, L⁴ is L^{4a}-C(O), wherein L^{4a} is C₅alkylene.

### Acyl Unit

In the compounds described herein, A is an acyl unit. In certain embodiments, the acyl unit “A” comprises a sulfur atom and is linked to the targeting moiety via a sulfur atom derived from the targeting moiety. In such instance, a dithio bond is formed between the acyl unit and the targeting moiety.

In certain embodiments, A is selected from

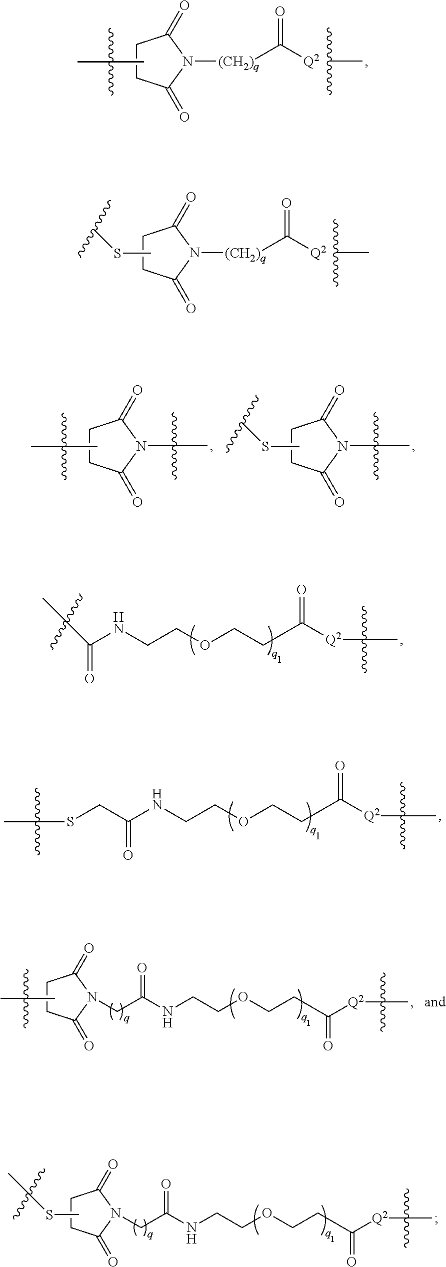

wherein Q² is NH or O, each q is independently an integer from 1 to 10, and each q₁ is independently an integer from 1 to 10. In some embodiments, q is an integer from 2 to 5, such as 2, 3, 4, or 5. In some embodiments, q₁ is an integer from 2 to 5, such as 2, 3, 4, or 5.

In certain embodiments, A is

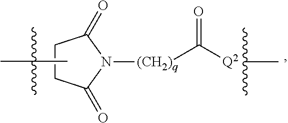

wherein Q² is NH or O and q is the integer 1, 2, 3, 4, 5, 6, 7, 8, 9, or 10. In certain instance, q is a number from 2 to 5, such as 2, 3, 4, or 5.

In certain embodiments, A is

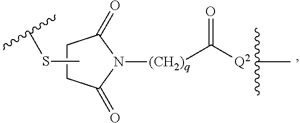

wherein Q² is NH or O and q is the integer 1, 2, 3, 4, 5, 6, 7, 8, 9, or 10. In certain instance, q is a number from 2 to 5, such as 2, 3, 4, or 5.
In certain embodiments, A is selected from

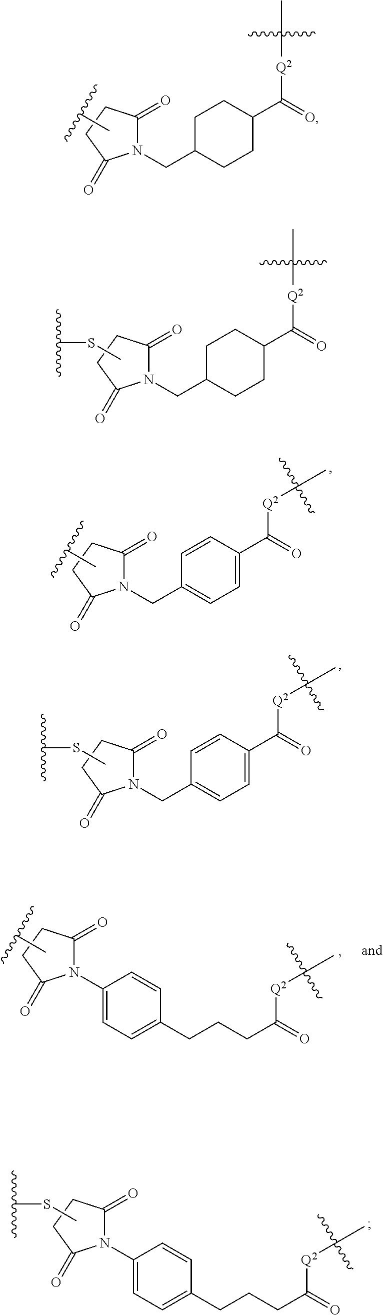

wherein Q² is NH or 0.

### Drug Moiety

The drug conjugates of the present embodiments are effective for the usual purposes for which the corresponding drugs are effective, and have superior efficacy because of the ability, inherent in the targeting moiety, to transport the drug to the desired cell where it is of particular benefit.

The preferred drugs for use in the present embodiments are cytotoxic drugs, such as those which are used for cancer therapy. Such drugs include, in general, DNA damaging agents, anti-metabolites, natural products and their analogs. Certain classes of cytotoxic agents include, for example, the enzyme inhibitors such as dihydrofolate reductase inhibitors, thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, differentiation inducers, and taxols. Certain useful members of those classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, carminomycin, aminopterin, tallysomycin, podophyllotoxin and podophyllotoxin derivatives such as etoposide or etoposide phosphate, vinblastine, vincristine, vindesine, taxol, taxotere retinoic acid, butyric acid, N⁸-acetyl spermidine, camptothecin, and their analogues. Other drugs include dolastatin and duocarmycin.

One skilled in the art may make chemical modifications to the desired compound in order to make reactions of that compound more convenient for purposes of preparing conjugates of the disclosure.

In certain embodiments, D is a drug moiety having a chemically reactive functional group by means of which the drug is bonded to L¹ or X. In certain instances, the functional group is selected from a primary amine, a secondary amine, hydroxyl, and sulfhydryl. In certain instances, the functional group is a primary amine or a secondary amine. In certain instances, the functional group is hydroxyl. In certain instances, the functional group is sulfhydryl.

As discussed above, the hydrophilic self-immolative linker will typically terminate with an oxycarbonyl group (—O—C(O)—). Thus, an amino-containing drug moiety would readily react with the oxycarbonyl group to form a carbamate group. In certain embodiments, D is an amino-containing drug moiety, wherein the drug is connected to L¹ or X through the amino group. However, if the drug moiety does not contain an amino group, the second self-immolative linker or cyclization self-elimination linker of L¹ can provide design potential for a wider variety of moieties that can be used. In certain embodiments, D is a hydroxyl-containing or sulfhydryl-containing drug moiety, wherein the drug is connected to L¹ through the hydroxyl or sulfhydryl group.

Representative amino-containing drugs include mitomycin-C, mitomycin-A, daunorubicin, doxorubicin, aminopterin, actinomycin, bleomycin, 9-amino camptothecin, N⁸-acetyl spermidine, 1-(2-chloroethyl)-1,2-dimethanesulfonyl hydrazide, tallysomycin, cytarabine, dolastatin and derivatives thereof. Amino-containing drugs also include amino derivatives of drugs that do not naturally contain an amino group. In certain embodiments, D is duocarmycin, dolastatin, tubulysin, doxorubicin (DOX), paclitaxel, or mitomycin C (MMC), or amino derivatives thereof.

Representative hydroxyl-containing drugs include etoposide, camptothecin, taxol, esperamicin, 1,8-dihydroxy-bicyclo[7.3.1]trideca-4-9-diene-2,6-diyne-13-one, (U.S. Pat. No. 5,198,560), podophyllotoxin, anguidine, vincristine, vinblastine, morpholine-doxorubicin, n-(5,5-diacetoxy-pentyl) doxorubicin, duocarmycin, and derivatives thereof.

Representative sulfhydryl-containing drugs include esperamicin and 6-mercaptopurine, and derivatives thereof.

A certain group of cytotoxic agents for use as drugs in the present embodiments include drugs of the following formulae:

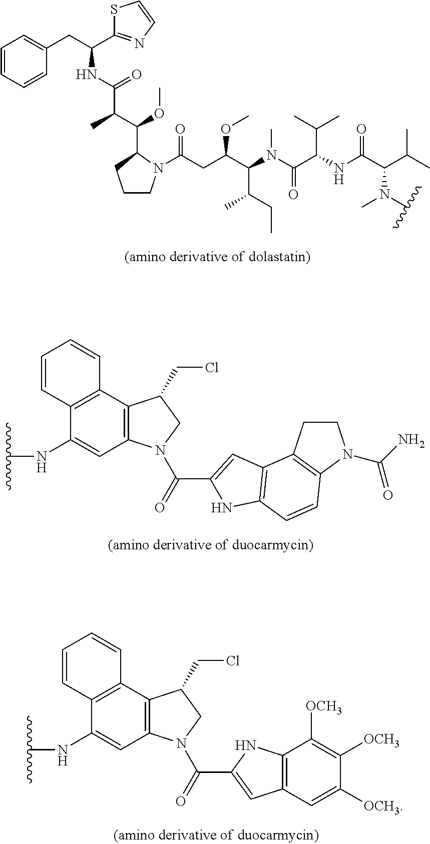 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C)C(C)C ; *Nc1cc2c(c3ccccc13)[C@H](CCl)CN2C(=O)c1cc2c3c(ccc2[nH]1)N(C(N)=O)CC3 ; *Nc1cc2c(c3ccccc13)[C@H](CCl)CN2C(=O)c1cc2cc(OC)c(OC)c(OC)c2[nH]1]

### Targeting Moiety

A targeting moiety as described in the present disclosure refers to a moiety or molecule that specifically binds, complexes with, reacts with, or associates with a given cell population. For example, a targeting moiety may specifically bind, complex with, react with, or associate with a receptive moiety or receptor associated with a given cell population (e.g., a given cell population sought to be therapeutically treated or otherwise biologically modified). In a conjugate described herein, a targeting moiety described herein is linked via a linker to a drug moiety in the conjugate. In some embodiments, the targeting moiety is capable of delivering a drug moiety (e.g., a drug moiety used for therapeutic purpose) to a particular target cell population which the targeting moiety binds, complexes with, reacts with, or associates with.

The targeting moiety may include, for example, large molecular weight proteins such as, for example, antibodies, smaller molecular weight proteins, polypeptide or peptide, and non-peptidyl moiety. A protein, polypeptide, or peptide moiety described herein may include, for example, transferrin, serum albumin, epidermal growth factors (“EGF”), bombesin, gastrin, gastrin-releasing peptide, platelet-derived growth factor, IL-2, IL-6, tumor growth factors (“TGF”), such as TGF-α, and TGF-β, vaccinia growth factor (“VGF”), insulin and insulin-like growth factors I and II. Non-peptidyl moiety may include, for example, carbohydrates, lectins, and apoprotein from low density lipoprotein. A protein, an antibody, a polypeptide, or a peptide in certain embodiments may refer to its unmodified form, a form that has been modified for being used in a conjugate described herein such as being used to bond to a linker, or a moiety that is in a conjugate described herein.

In some embodiments, the targeting moiety is an antibody (or an antibody moiety or an antibody targeting moiety). In some embodiments, the targeting moiety comprises an antibody. In some embodiments, the targeting moiety comprises sulfhydryl (—SH) group (e.g., a free reactive sulfhydryl (—SH) group) or can be modified to contain such a sulfhydryl group. In some embodiments, the targeting moiety comprises an antibody with a sulfhydryl group (e.g., a free reactive sulfhydryl group). In some embodiments, the targeting moiety comprises a free thiol group such as an antibody with a free thiol group or can be modified to contain such a thio group. In some embodiments, the targeting moiety comprising a sulfhydryl group or thiol group bonds to a linker via the sulfur atom in the sulfhydryl group.

In some embodiments, the targeting moiety (e.g., an antibody targeting moiety) has one or more attachment sites for linking to the drug moiety. For example, a targeting moiety T (e.g., an antibody) can have multiple sites (e.g., multiple sulfhydryl groups) for linking to a linker-drug moiety (e.g., A-L⁴-L³-L²-X-L¹-D where A is suitable for bonding to a sulfhydryl group of the targeting antibody). In some embodiments, the targeting moiety can have 1 to 20 sites of attachment. In some embodiments, the targeting moiety can have 1 to 20, 1 to 10, 1 to 8, 1 to 6, 1 to 4, 2 to 8, 2 to 6, or 2 to 4 sites of attachment. In some embodiments, the targeting moiety has 1, 2, 3, 4, 5, 6, 7, 8, 9, 10, 11, 12, 13, 14, 15, 16, 17, 18, 19, or 20 sites of attachment. In some embodiments, the targeting moiety has 1, 2, 3, 4, 5, 6, 7, or 8 sites of attachment. In some embodiments, the targeting moiety has 2 sites of attachment. In some embodiments, the targeting moiety has 1 site of attachment. In some embodiments, the targeting moiety has 4 sites of attachment. In some instances, certain potential sites of attachment may not be accessible for bonding to a drug moiety. Thus, the number of attachment sites in a targeting moiety T may results in a drug conjugate that has fewer number of drug moieties attached than the number of potential sites of attachment. In some embodiments, one or more of the sites of attachment may be accessible for bonding a drug moiety. For example, an antibody targeting moiety can have one or two sulfhydryl groups on each chain of the antibody accessible for bonding to drug moiety via a linker.

In some embodiments, the targeting moiety is an antibody or an antibody targeting moiety. An antibody described herein refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also antigen-binding fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An antibody included or used in a targeting moiety described herein (or an antibody targeting moiety) can encompass monoclonal antibodies, polyclonal antibodies, antibody fragments (e.g., Fab, Fab′, F(ab′)₂, Fv, Fc, etc.), chimeric antibodies, humanized antibodies, human antibodies (e.g., fully human antibodies), single chain (ScFv), bispecific antibodies, multispecific antibodies, mutants thereof, fusion proteins comprising an antibody portion, and any other modified configuration of the immunoglobulin molecule that comprises an antigen recognition site of the required specificity. The antibodies may be murine, rat, camel, human, or any other origin (including humanized antibodies). In some embodiments, an antibody used in a targeting moiety described herein (or an antibody targeting moiety) is any one of the following: bispecific antibody, multispecific, single-chain, bifunctional, and chimeric and humanized molecules having affinity for a polypeptide conferred by at least one hypervariable region (HVR) or complementarity determining region (CDR) of the antibody. Antibodies used in the present disclosure also include single domain antibodies which are either the variable domain of an antibody heavy chain or the variable domain of an antibody light chain. Holt et al., Trends Biotechnol. 21:484-490, 2003. Methods of making domain antibodies comprising either the variable domain of an antibody heavy chain or the variable domain of an antibody light chain, containing three of the six naturally occurring HVRs or CDRs from an antibody, are also known in the art. See, e.g., Muyldermans, Rev. Mol. Biotechnol. 74:277-302, 2001.

In some embodiments, an antibody included or used in a targeting moiety described herein (or an antibody targeting moiety) is a monoclonal antibody. As used herein, a monoclonal antibody refers to an antibody of substantially homogeneous antibodies, i.e., the individual antibodies comprising the population are identical except for possible naturally-occurring mutations that may be present in minor amounts. Furthermore, in contrast to polyclonal antibody preparations, which typically include different antibodies directed against different determinants (epitopes), monoclonal antibody is not a mixture of discrete antibodies. The modifier “monoclonal” indicates the character of the antibody as being obtained from a substantially homogeneous population of antibodies, and is not to be construed as requiring production of the antibody by any particular method. For example, the monoclonal antibodies used in the present disclosure may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

In some embodiments, an antibody included or used in a targeting moiety described herein (or an antibody targeting moiety) is a chimeric antibody. As used herein, a chimeric antibody refers to an antibody having a variable region or part of variable region from a first species and a constant region from a second species. An intact chimeric antibody comprises two copies of a chimeric light chain and two copies of a chimeric heavy chain. The production of chimeric antibodies is known in the art (Cabilly et al. (1984), Proc. Natl. Acad. Sci. USA, 81:3273-3277; Harlow and Lane (1988), Antibodies: a Laboratory Manual, Cold Spring Harbor Laboratory). Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non-human host organisms in combination with constant regions derived from, for example, human cell preparations. While the variable region has the advantage of ease of preparation, and the specificity is not affected by its source, the constant region being human is less likely to elicit an immune response from a human subject when the antibodies are injected than would the constant region from a non-human source. However, the definition is not limited to this particular example.

In some embodiments, an antibody included or used in a targeting moiety described herein (or an antibody targeting moiety) is a humanized antibody. As used herein, humanized antibodies refer to forms of non-human (e.g. murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a HVR or CDR of the recipient are replaced by residues from a HVR or CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported HVR or CDR or framework sequences, but are included to further refine and optimize antibody performance. In general, the humanized antibody will comprise substantially all of at least one, and typically two, variable domains, in which all or substantially all of the HVR or CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more HVRs or CDRs (one, two, three, four, five, six) which are altered with respect to the original antibody, which are also termed one or more HVRs or CDRs “derived from” one or more HVRs or CDRs from the original antibody.

In some embodiments, an antibody included or used in a targeting moiety described herein (or an antibody targeting moiety) is a human antibody. As used herein, a human antibody means an antibody having an amino acid sequence corresponding to that of an antibody produced by a human and/or has been made using any of the techniques for making human antibodies known in the art. A human antibody used herein includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

The human epidermal growth factor 2 protein, HER2 (ErbB2) is a receptor tyrosine kinase that is known to play critical roles in both development and oncogenesis. In some embodiments the antibody included or used in a targeting moiety described herein (or an antibody targeting moiety) specifically binds to HER2. In further embodiments, the anti-HER2 antibody is a monoclonal or humanized antibody. The humanized monoclonal anti-HER2 antibody trastuzumab (HERCEPTIN®) is currently used to treat HER2-positive cancers. In some embodiments, the antibody included or used in a targeting moiety described herein is trastuzumab. Other anti-HER2 antibodies, including pertuzumab (PERJETA®) and margetuximab, are also known in the art. In some embodiments, the antibody included or used in a targeting moiety described herein is pertuzumab. In some embodiments, the antibody included or used in a targeting moiety described herein margetuximab.

In some embodiments, the anti-HER2 antibody comprises a light chain variable region comprising one, two or three HVRs (or CDRs) from SEQ ID NO:7 and/or a heavy chain variable region comprising one, two or three HVRs (or CDRs) from SEQ ID NO:8. In some embodiments, the antibody comprises a light chain variable region comprising the three HVRs (or CDRs) from SEQ ID NO:7 and/or a heavy chain variable region comprising the three HVRs (or CDRs) from SEQ ID NO:8. In some embodiments, the antibody comprises a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:7, and/or a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:8. In some embodiments, the antibody comprises a light chain variable region comprising the amino acid sequence of SEQ ID NO:7 and/or a heavy chain variable region comprising amino acid sequence of SEQ ID NO:8.

In some embodiments, the anti-HER2 antibody comprises a light chain variable region comprising one, two or three HVRs (or CDRs) from SEQ ID NO:12 and/or a heavy chain variable region comprising one, two or three HVRs (or CDRs) from SEQ ID NO:13. In some embodiments, the antibody comprises a light chain variable region comprising the three HVRs (or CDRs) from SEQ ID NO:12 and/or a heavy chain variable region comprising the three HVRs (or CDRs) from SEQ ID NO:13. In some embodiments, the antibody comprises a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:12, and/or a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:13. In some embodiments, the antibody comprises a light chain variable region comprising the amino acid sequence of SEQ ID NO:12 and/or a heavy chain variable region comprising amino acid sequence of SEQ ID NO:13.

In some embodiments, the anti-HER2 antibody comprises a light chain variable region comprising one, two or three HVRs (or CDRs) from SEQ ID NO:14 and/or a heavy chain variable region comprising one, two or three HVRs (or CDRs) from SEQ ID NO:15. In some embodiments, the antibody comprises a light chain variable region comprising the three HVRs (or CDRs) from SEQ ID NO:14 and/or a heavy chain variable region comprising the three HVRs (or CDRs) from SEQ ID NO:15. In some embodiments, the antibody comprises a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:14, and/or a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:15. In some embodiments, the antibody comprises a light chain variable region comprising the amino acid sequence of SEQ ID NO:14 and/or a heavy chain variable region comprising amino acid sequence of SEQ ID NO:15.

TABLE 1
Sequence ID NOs. of anti-HER2 ADCs

| SEQ | |
|---|---|
| ID NO. | Description |
| 1 | Human kappa light chain constant domain sequence |
| 2 | Human IgG1 heavy chain constant domain sequence |
| 3 | Human IgG2 heavy chain constant domain sequence |
| 4 | Human IgG3 heavy chain constant domain sequence |
| 5 | Human IgG4 heavy chain constant domain sequence |
| 6 | Amino acid sequence of hIgG4-S228P heavy chain constant |
| | region |
| 7 | Amino acid sequence of Trastuzumab (HERCEPTIN ®, Roche |
| | Inc.) light chain variable region |
| 8 | Amino acid sequence of Trastuzumab (HERCEPTIN ®, Roche |
| | Inc.) heavy chain variable region |
| 9 | Amino acid sequence of Trastuzumab (HERCEPTIN ®, Roche |
| | Inc.) light chain comprising human kappa constant domain |
| 10 | Amino acid sequence of Trastuzumab (HERCEPTIN ®, Roche |
| | Inc.) heavy chain comprising human IgG1 constant domain |
| 11 | Amino acid sequence of Trastuzumab (HERCEPTIN ®, Roche |
| | Inc.) heavy chain comprising human IgG4-S228P constant |
| | domain |
| 12 | Amino acid sequence of Pertuzumab (PERJETA ®, Roche |
| | Inc.) light chain variable region |
| 13 | Amino acid sequence of Pertuzumab (PERJETA ®, Roche |
| | Inc.) heavy chain variable region |
| 14 | Amino acid sequence of Margetuximab (Macrogenics |
| | Inc.) light chain variable region |
| 15 | Amino acid sequence of Margetuximab (Macrogenics |
| | Inc.) heavy chain variable region |

Human kappa light chain constant domain sequence
(SEQ ID NO. 1)
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
Human IgG1 heavy chain constant domain sequence
(SEQ ID NO. 2)
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
Human IgG2 heavy chain constant domain sequence
(SEQ ID NO. 3)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPAAAPSV
FLFPPKPKDTLMISRTPEVTCVVVDVSHEDPEVQFNWYVDGVEVHNAKTKPREEQFNST
FRVVSVLTVVHQDWLNGKEYKCKVSNKGLPAPIEKTISKTKGQPREPQVYTLPPSREEM
TKNQVSLTCLVKGFYPSDIAVEWESNGQPENNYKTTPPMLDSDGSFFLYSKLTVDKSRW
QQGNVFSCSVMHEALHNHYTQKSLSLSPSK
Human IgG3 heavy chain constant domain sequence
(SEQ ID NO. 4)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDTLMI
SRTPEVTCVVVDVSHEDPEVQFKWYVDGVEVHNAKTKPREEQYNSTFRVVSVLTVLHQ
DWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMH
EALHNRFTQKSLSLSPGK
Human IgG4 heavy chain constant domain sequence
(SEQ ID NO. 5)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPSCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLGK
Amino acid sequence of hIgG4-S228P
(SEQ ID NO. 6)
ASTKGPSVFPLAPCSRSTSESTAALGCLVKDYFPEPVTVSWNSGALTSGVHTFPAVLQSS
GLYSLSSVVTVPSSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVYTLPPSQEEM
TKNQVSLTCLVKGFYPSDIAVEWESNGQPENNYKTTPPVLDSDGSFFLYSRLTVDKSRW
QEGNVFSCSVMHEALHNHYTQKSLSLSLGK
Amino acid sequence of Trastuzumab (HERCEPTIN ®, Roche Inc.)
light chain variable region
(SEQ ID NO. 7)
DIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIK
Amino acid sequence of Trastuzumab (HERCEPTIN ®, Roche Inc.)
heavy chain variable region
(SEQ ID NO. 8)
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGT
LVTVSS
Amino acid sequence of Trastuzumab (HERCEPTIN ®, Roche Inc.)
light chain comprising human kappa constant domain
(SEQ ID NO. 9)
DIQMTQSPSSLSASVGDRVTITCRASQDVNTAVAWYQQKPGKAPKLLIYSASFLYSGVP
SRFSGSRSGTDFTLTISSLQPEDFATYYCQQHYTTPPTFGQGTKVEIKRTVAAPSVFIFPPS
DEQLKSGTASVVCLLNNFYPREAKVQWKVDNALQSGNSQESVTEQDSKDSTYSLSSTL
TLSKADYEKHKVYACEVTHQGLSSPVTKSFNRGEC
Amino acid sequence of Trastuzumab (HERCEPTIN ®, Roche Inc.)
heavy chain comprising human IgG1 constant domain
(SEQ ID NO. 10)
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGT
LVTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQV
YTLPPSREEMTKNQVSLTCLVKGFYPSDIAVEWESNGQPENNYKTTPPVLDSDGSFFLYS
KLTVDKSRWQQGNVFSCSVMHEALHNHYTQKSLSLSPGK
Amino acid sequence of Trastuzumab (HERCEPTIN ®, Roche Inc.)
heavy chain comprising human IgG4-S228P constant domain
(SEQ ID NO. 11)
EVQLVESGGGLVQPGGSLRLSCAASGFNIKDTYIHWVRQAPGKGLEWVARIYPTNGYT
RYADSVKGRFTISADTSKNTAYLQMNSLRAEDTAVYYCSRWGGDGFYAMDYWGQGT
LVTVSSASTKGPSVFPLAPCSRSTSESTAALGCLVKDYFPEPVTVSWNSGALTSGVHTFP
AVLQSSGLYSLSSVVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQPREPQVYTLP
PSQEEMTKNQVSLTCLVKGFYPSDIAVEWESNGQPENNYKTTPPVLDSDGSFFLYSRLT
VDKSRWQEGNVFSCSVMHEALHNHYTQKSLSLSLGK
Amino acid sequence of Pertuzumab (PERJETA ®, Roche Inc.)
light chain variable region
(SEQ ID NO. 12)
DIQMTQSPSSLSASVGDRVTITCKASQDVSIGVAWYQQKPGKAPKLLIYSASYRYTGVPS
RFSGSGSGTDFTLTISSLQPEDFATYYCQQYYIYPYTFGQGTKVEIK
Amino acid sequence of Pertuzumab (PERJETA ®, Roche Inc.)
heavy chain variable region
(SEQ ID NO. 13)
EVQLVESGGGLVQPGGSLRLSCAASGFTFTDYTMDWVRQAPGKGLEWVADVNPNSGG
SIYNQRFKGRFTLSVDRSKNTLYLQMNSLRAEDTAVYYCARNLGPSFYFDYWGQGTLV
TVSS
Amino acid sequence of Margetuximab (Macrogenics Inc.)
light chain variable region
(SEQ ID NO. 14)
DIVMTQSHKFMSTSVGDRVSITCKASQDVNTAVAWYQQKPGHSPKLLIYSASFRYTGVP
DRFTGSRSGTDFTFTISSVQAEDLAVYYCQQHYTTPPTFGGGTKVEIK
Amino acid sequence of Margetuximab (Macrogenics Inc.)
heavy chain variable region
(SEQ ID NO. 15)
QVQLQQSGPELVKPGASLKLSCTASGFNIKDTYIHWVKQRPEQGLEWIGRIYPTNGYTR
YDPKFQDKATITADTSSNTAYLQVSRLTSEDTAVYYCSRWGGDGFYAMDYWGQGASV
TVSS

TABLE 2
Amino Acid Sequences of CDRs of anti-HER2
Antibodies Trastuzumab, Pertuzumab, and
Margetuximab

| | Trastuzumab | Pertuzumab | Margetuximab |
|---|---|---|---|
| CDR-H1 | DTYIH | DYTMD | DTYIH |
| | (SEQ ID NO: | (SEQ ID NO: 22) | (SEQ ID NO: 28) |
| | 16) | | |
| CDR-H2 | RIYPTNGYT | DVNPNSGGSIYNQRF | RIYPTNGYTRYDPKF |
| | RYADSVKG | KG | QD |
| | (SEQ ID NO: | (SEQ ID NO: 23) | (SEQ ID NO: 29) |
| | 17) | | |
| CDR-H3 | WGGDGFYAMDY | NLGPSFYFDY | WGGDGFYAMDY |
| | (SEQ ID NO: | (SEQ ID NO: 24) | (SEQ ID NO: 30) |
| | 18) | | |
| CDR-L1 | RASQDVNTAVA | KASQDVSIGVA | KASQDVNTAVA |
| | (SEQ ID NO: | (SEQ ID NO: 25) | (SEQ ID NO: 31) |
| | 19) | | |
| CDR-L2 | SASFLYS | SASYRYT | SASFRYT |
| | (SEQ ID NO: | (SEQ ID NO: 26) | (SEQ ID NO: 32) |
| | 20) | | |
| CDR-L3 | QQHYTTPPT | QQYYIYPYT | QQHYTTPPT |
| | (SEQ ID NO: | (SEQ ID NO: 27) | (SEQ ID NO: 33) |
| | 21) | | |

In some embodiments, the antibody included or used in a targeting moiety described herein is modified. In some embodiments, the modification is an engineered cysteine substitution.

In some embodiments, the engineered cysteine substitutions occur on the IgG heavy chain of the antibody. In some embodiments, the engineered cysteine substitutions occur at specific positions on the IgG heavy chain of the antibody. In some embodiments, the amino acid positions on the IgG heavy chain that may have engineered cysteine substitutions include (EU numbering) 118-215, 234, 235, 236, 237, 238, 239, 246, 248, 249, 254, 265, 267, 269, 270, 273, 276, 278, 279, 282, 283, 284, 286, 287, 289, 292, 293, 294, 297, 298, 299, 300, 302, 303, 312, 314, 315, 318, 320, 324, 326, 327, 330, 332, 333, 334, 335, 336, 337, 339, 341447. The above-disclosed amino acid positions are described in US 2012/0148580 A1; WO 2013/093809 A1; US 2009/0258420 A1; U.S. Pat. No. 7,521,541 B2; U.S. Pat. No. 7,855,275 B2; US 2011/0137017 A1; US 2012/0213705 A1; US 2011/0033378 A1; U.S. Pat. No. 8,455,622 B2 which are herein incorporated by reference in their entirety. Additional positions on the IgG heavy chain that can be engineered cysteine for site-specific conjugation include (EU numbering) 121, 122, 124, 125, 126, 129, 159, 187, 188, 190, 191, 193, 197, 199, 201, 202, 203, 205, 207, 208, 209, 211, 212, 215, 295, 296, 301.

In some embodiments, the engineered cysteine substitutions occur on the IgG light chain of the antibody. In some embodiments, the engineered cysteine substitutions occur at specific positions on the IgG light chain of the antibody. In some embodiments, the amino acid positions on the IgG light chain that may have engineered cysteine substitutions include (Kabat numbering) 108-211, as described in WO 2013/093809 A1; US 2009/0258420 A1; U.S. Pat. No. 7,855,275 B2; U.S. Pat. No. 8,455,622 B2, which are herein incorporated by reference in their entirety. Additional positions on the IgG light chain that can be engineered cysteine for site-specific conjugation include (Kabat numbering) 112, 114, 115, 116, 147, 195, 199, 200, 201, 202, 203, 206, 207, 208, 209, 210.

### Therapeutic Applications

In some embodiments, the present disclosure provide a method of killing a cell by administering to the cell a sufficiently lethal amount of the compounds discussed herein. In some embodiments, the cell that is killed is a cancer cell. In some embodiments, the cell is a breast cancer cell, or a gastric cancer cell or an ovarian cancer cell. In some embodiments the presently disclosed method for killing a cell may be performed in vitro. In some embodiments, the method for killing a cell may be performed in vivo.

In some embodiments, the compounds discussed herein may be administered in an effective dose as part of a therapeutic regimen in the treatment of a disease or disorder in a subject. In some embodiments, the present disclosure provides a method for the treatment of cancer in an individual in need thereof comprising administering to the individual an effective dose of the compounds disclosed herein. In some embodiments, the effective dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations, for one or several or many days, depending on the mode of administration and the factors discussed above.

In some embodiments, the compounds of the present disclosure may be administered in combination with other therapeutic compounds. In some embodiments, the other therapeutic compounds are anti-cancer drugs or chemotherapeutics. A person of ordinary skill in the art will be familiar with a wide variety of cancer chemotherapeutics. In some embodiments, the compounds of the present disclosure may be administered in combination with other forms of cancer therapy, such as (but not limited to) radiation therapy.

In some embodiments use of the presently disclosed method of treating cancer leads to beneficial or desired clinical results, including but not limited to reducing the proliferation of (or destroying) cancerous cells, decreasing symptoms resulting from the disease, increasing the quality of life of those suffering from the disease, and/or delaying development of the disease. As is evident to one skilled in the art, a sufficient or significant delay can, in effect, encompass prevention, in that the individual does not develop the cancer. For example, a late stage cancer, such as development of metastasis, may be delayed.

In some embodiments, the targeting moiety of the compounds described herein specifically binds to cancer cells. Exemplary but non-limiting examples of cancer-binding targeting moieties include anti-CD20 antibodies, anti-CD30 antibodies and anti-HER2 antibodies. In further embodiments, the drug moiety of the compounds discussed herein is a drug that is effective in treating cancer. Non-limiting examples of such drugs include mitomycin-C, mitomycin-A, daunorubicin, doxorubicin, aminopterin, actinomycin, bleomycin, 9-amino camptothecin, N⁸-acetyl spermidine, 1-(2-chloroethyl)-1,2-dimethanesulfonyl hydrazide, tallysomycin, cytarabine, dolastatin and derivatives thereof.

The following examples are offered to illustrate but not limit the utility of the present disclosure.

### Example 1

### Materials and Methods for Example 2

### Synthesis of Linker-Drug

Synthesis of Compound Tap-18H is shown below in the scheme. Synthesis of intermediate Compounds M and O are also shown below in the schemes.

Synthesis of Compound TAP-18H

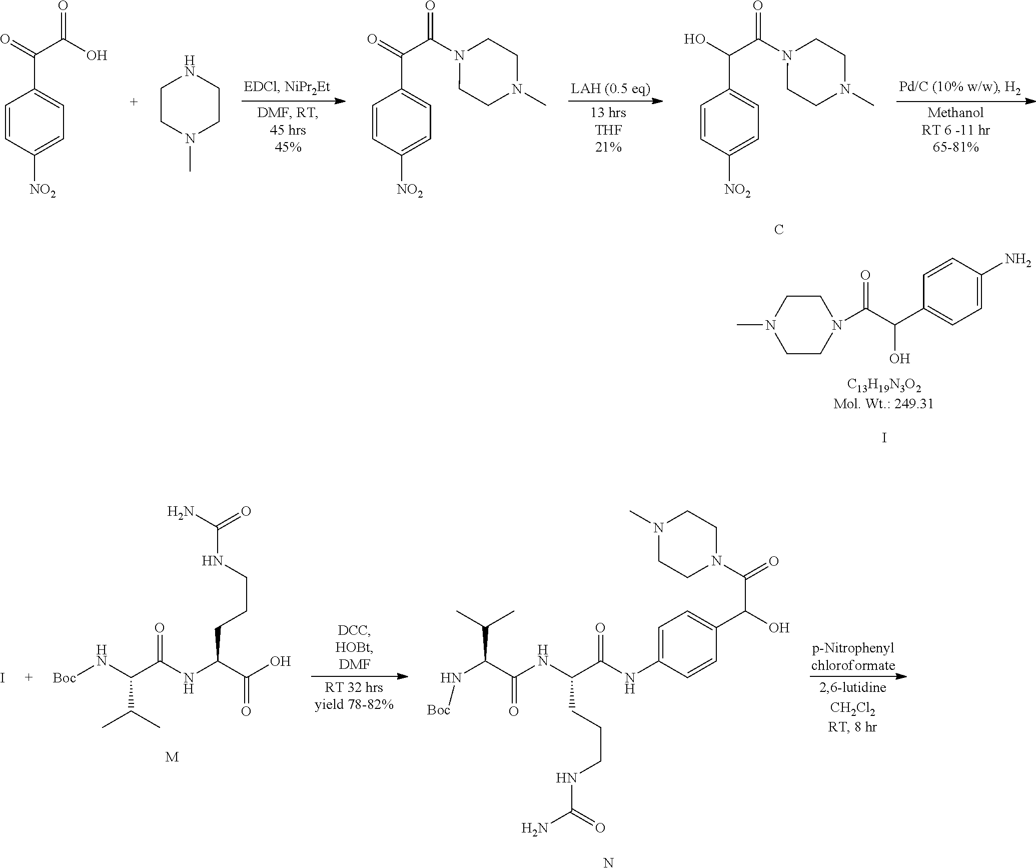

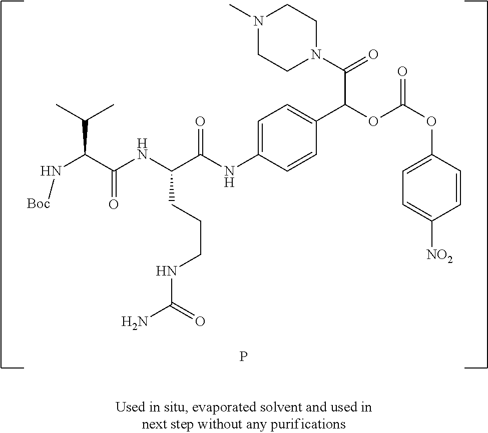

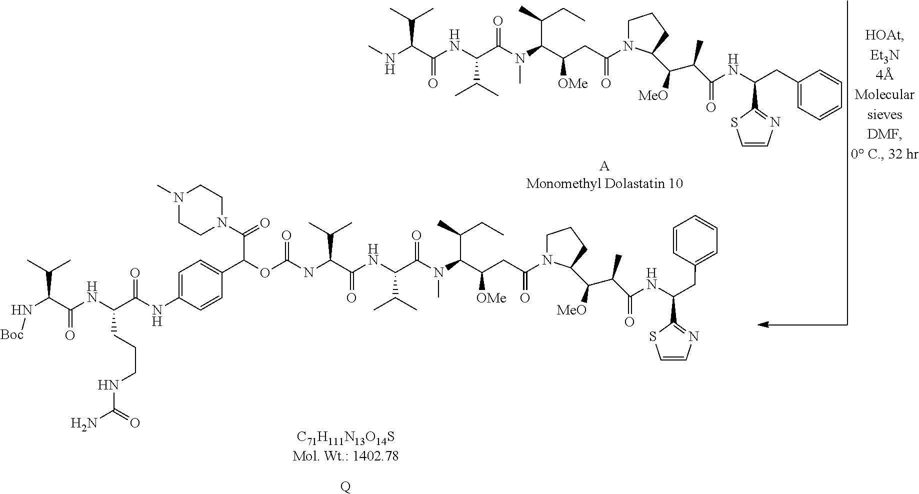

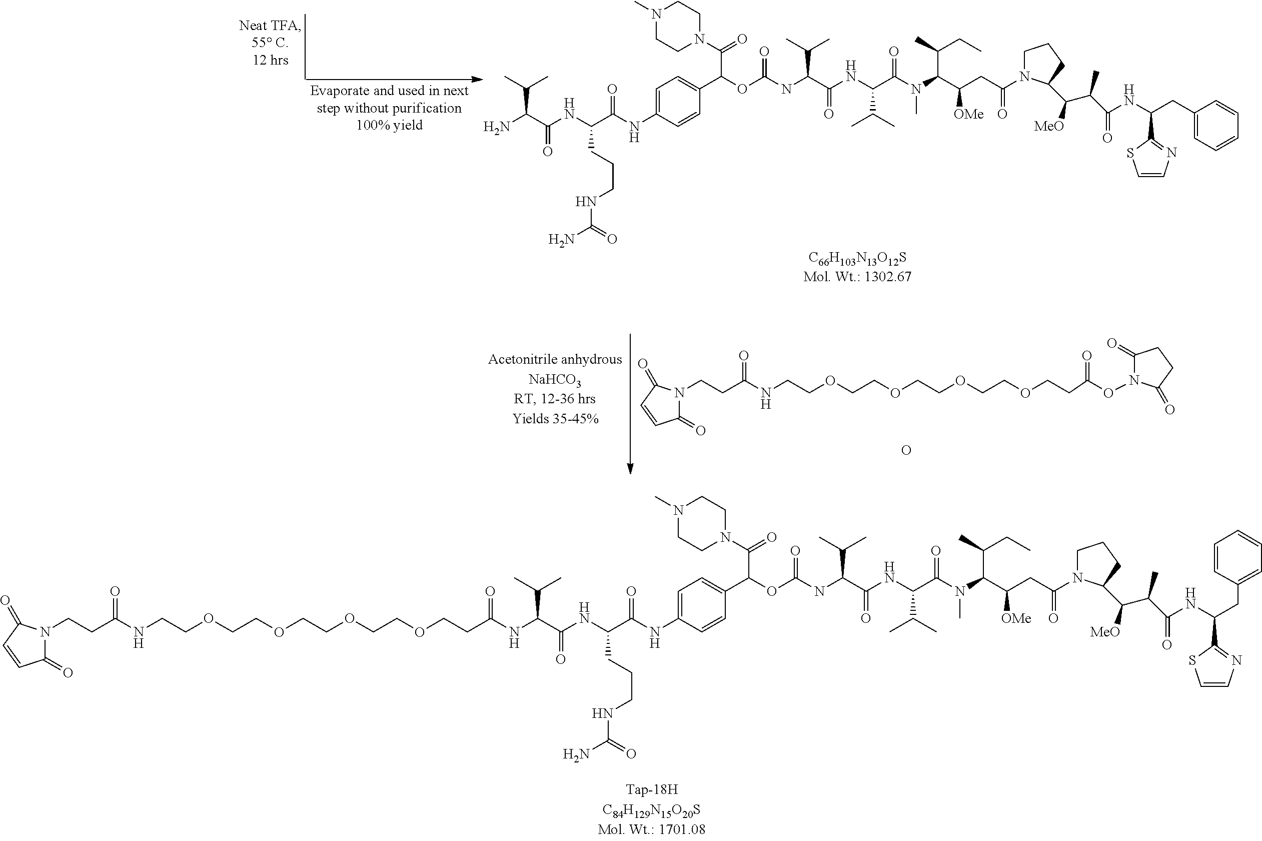

Synthesis of Compound M

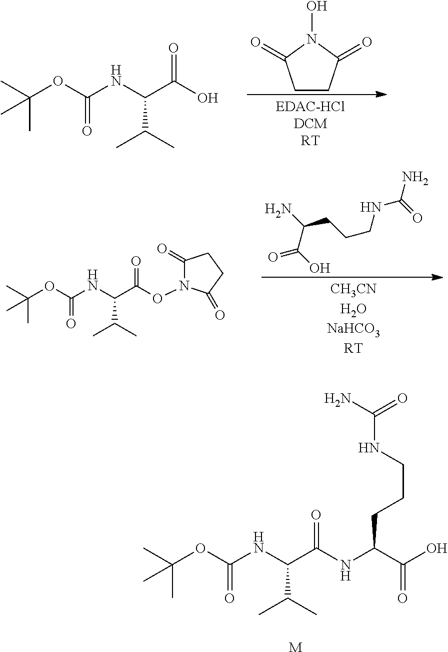

Synthesis of Compound O

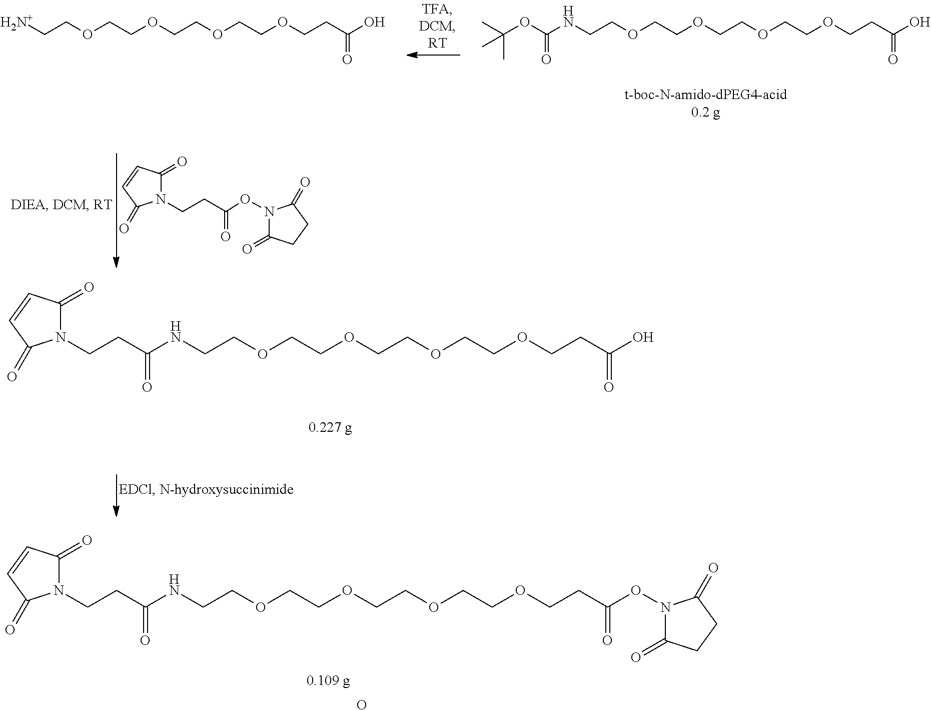 [SMILES: [NH3+]CCOCCOCCOCCOCCC(=O)O ; CC(C)(C)OC(=O)NCCOCCOCCOCCOCCC(=O)O ; O=C(CCN1C(=O)C=CC1=O)ON1C(=O)CCC1=O ; O=C(O)CCOCCOCCOCCOCCNC(=O)CCN1C(=O)C=CC1=O ; O=C(CCN1C(=O)C=CC1=O)NCCOCCOCCOCCOCCC(=O)ON1C(=O)CCC1=O ; O]

Referring to the scheme of synthesis of Compound Tap-18H, commercially available 4-nitrophenylglyoxylic acid was condensed with N-methylpiperazine using either PCl₅, or EDCI and NiPr₂Et in DMF, or 2-chloro-4,6-dimethoxy-1,3,5-triazine in CH₂Cl₂ and N-methylmorpholine as coupling agent to produce the desired ketoamide. In a typical procedure, a solution of 2-chloro-4,6-dimethoxy-1,3,5-triazine (5 mmol) in CH₂Cl₂(20 ml), N-methylmorpholine (15 mmol) was added at 0-5° C. under continuous stirring. A white suspension was formed after 30-40 minutes and to this mixture 4-nitrophenylglyoxylic acid in CH₂Cl₂ (10 ml) was added, resulting in the formation of a clear solution. After stirring the mixture for 1 hour, N-methylpiperazine (5 mmol) was added at room temperature. After completion of the reaction (TLC, 10 minutes), the mixture was washed with 10% aqueous NaHCO₃ solution (2×10 ml) followed by H₂O (3×10 ml). The organic layer was dried over anhydrous sodium sulfate and removal of the solvent under reduced pressure furnished a crude product which was further purified by recrystallization or column chromatography (pet. ether:ethyl acetate=8:2).

The ketoamide compound was further reduced by 0.5 equivalent amounts of LiAlH₄ in the presence of THF or DIBAL-H or sodium borohydride to produce the nitro Compound C. [B. P. Bandgar and S. S. Pandit, Tetrahedron Letters 44 (2003) 3855-3858] Nitro Compound C was reduced to aniline Compound I by either treatment with SnCl₂ or catalytic hydrogenation with Pd/C (10% w/w) as catalyst in methanol at room temperature for about 6-11 hours with yield from 65-81%. It could be obtained through the following procedures using MultiMaxIR system with an RB04-50 Reactor B. The reactor was filled initially with 35 ml of methanol, 0.03 mg of 10% Pd/C and 0.0252 mol of nitro Compound C and the hydrogen was add in the reactor up to pressure at 6.3 bar (H₂, const.).

Referring to the scheme of synthesis of Compound M, Boc-protected L-valine was treated with N-hydroxysuccinimide and EDAC-HCl in DCM or N-hydroxysuccinimide and EDC in DCM to give the succinimide ester. This activated ester was reacted with L-Citrulline and CH₃CN, H₂O, NaHCO₃ to furnish Boc-protected Compound M.

Referring to the scheme of synthesis of Compound Tap-18H, aniline Compound I was coupled with Boc-protected Compound M by means of either DCC/HOBt in DMF at room temperature for 32 hours to give Compound N (yield 78-82%), or with PS-carbodiimide, in which reaction the synthesis of Compound N was carried out starting from 100 mg of Compound M with 1.5 equivalents of aniline Compound 1 in the presence of two equivalents of PS-carbodiimide and 1.7 equivalents of HOBt in DCM for 24 hours. Analysis by LC/MS showed the peak with the desired mass and approximately 50-60% conversion.

The coupled product Compound N was then reacted with 4-nitrophenyl chloroformate in the presence of 2,6-lutidine in DCM at RT for 8 hours to yield carbonate Compound P, LC/MS showed the peak with the desired mass.

Treatment of carbonate Compound P with monomethyl Dolastatin 10 in the presence of HOAt and Et₃N in DMF resulted in the formation of Compound Q.

Referring to the scheme of synthesis of Compound O, β-alanine was treated with maleic anhydride in DMF and the acid so obtained was reacted with N-hydroxysuccinimide (NHS) under DCC coupling to give NHS-ester. The BOC protective group in commercially available t-boc-N-amido-dPEG₄-acid was removed by treatment with TFA to give the TFA salt of the amine, which was reacted with previously synthesized NHS ester. The carboxylic acid so obtained was isolated and was coupled with N-hydroxysuccinimide using EDCI to furnish NHS ester Compound O.

Referring to the scheme of synthesis of Compound Tap-18H, the Boc-group in Compound Q was removed with TFA and the free amine was coupled with NHS ester Compound O in anhydrous acetonitrile and NaHCO₃ at room temperature for 12-36 hours to produce the final product Tap-18H with yield of 35-45%.

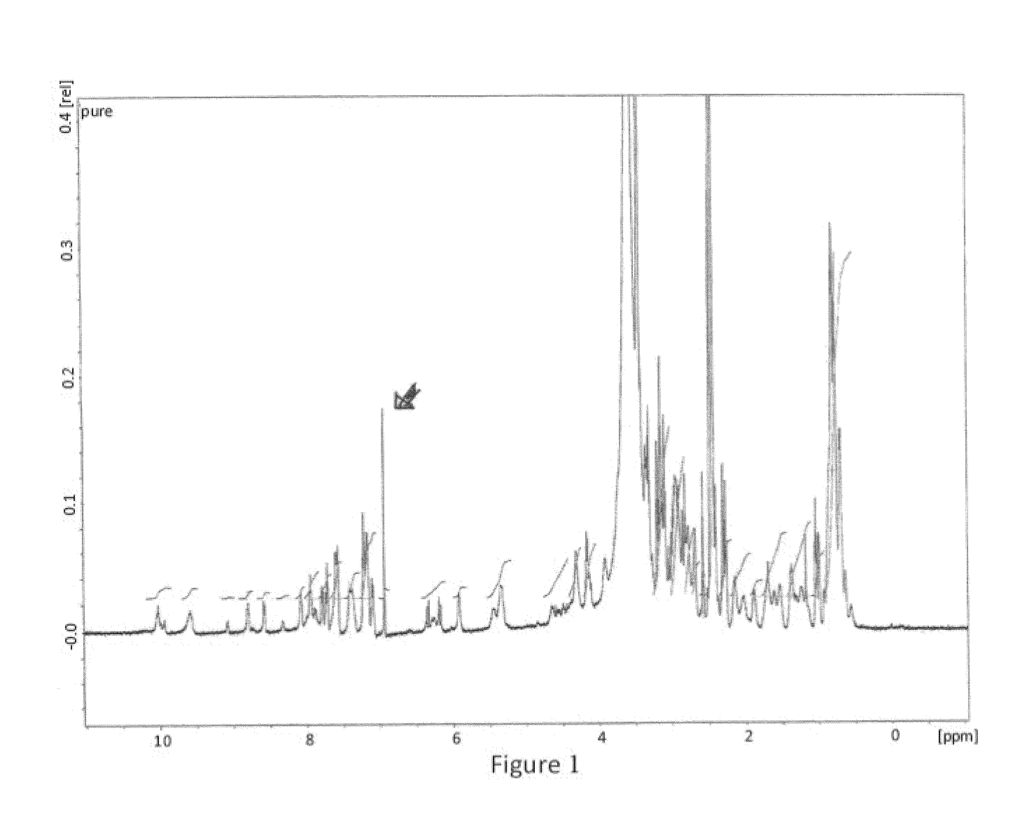

FIG. 1 shows an NMR spectrum of Tap-18H.

Synthesis of Compound TAP-18Hr1

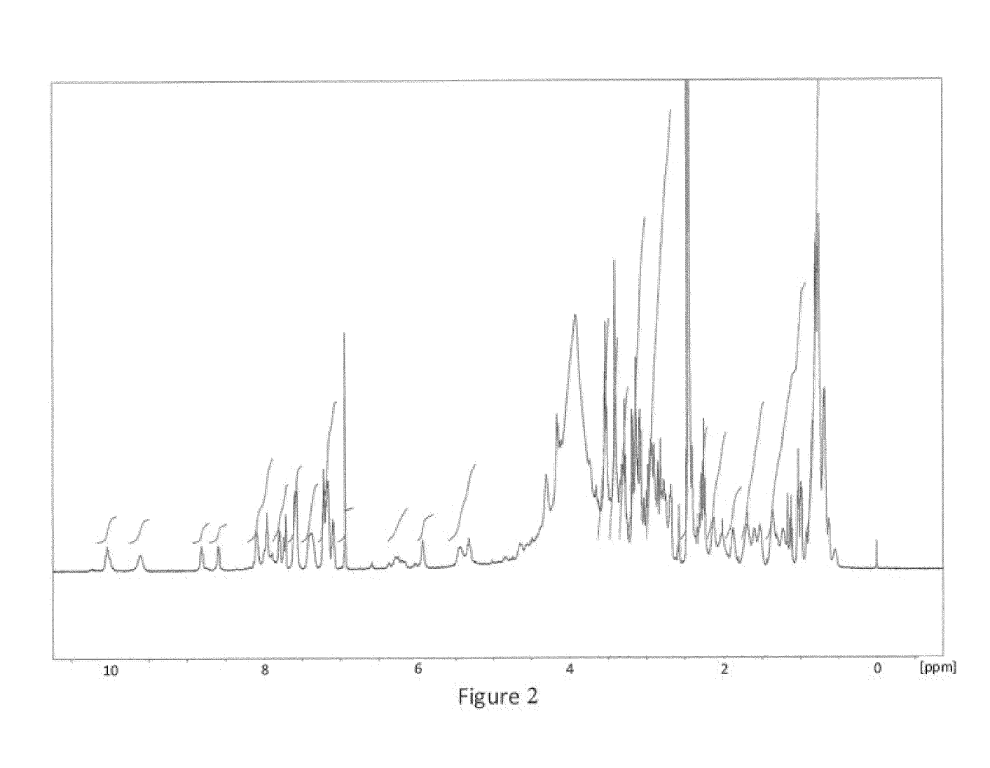

Tap-18Hr1 was synthesized with the formula shown below. FIG. 2 shows NMR spectrum of Tap-18Hr1.

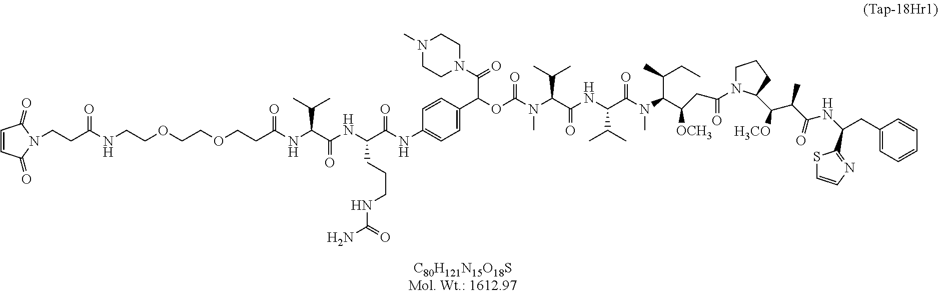 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C(=O)OC(C(=O)N1CCN(C)CC1)c1ccc(NC(=O)[C@H](CCCNC(N)=O)NC(=O)[C@@H](NC(=O)CCOCCOCCNC(=O)CCN2C(=O)C=CC2=O)C(C)C)cc1)C(C)C]

Synthesis of Compound TAP-18Hr2

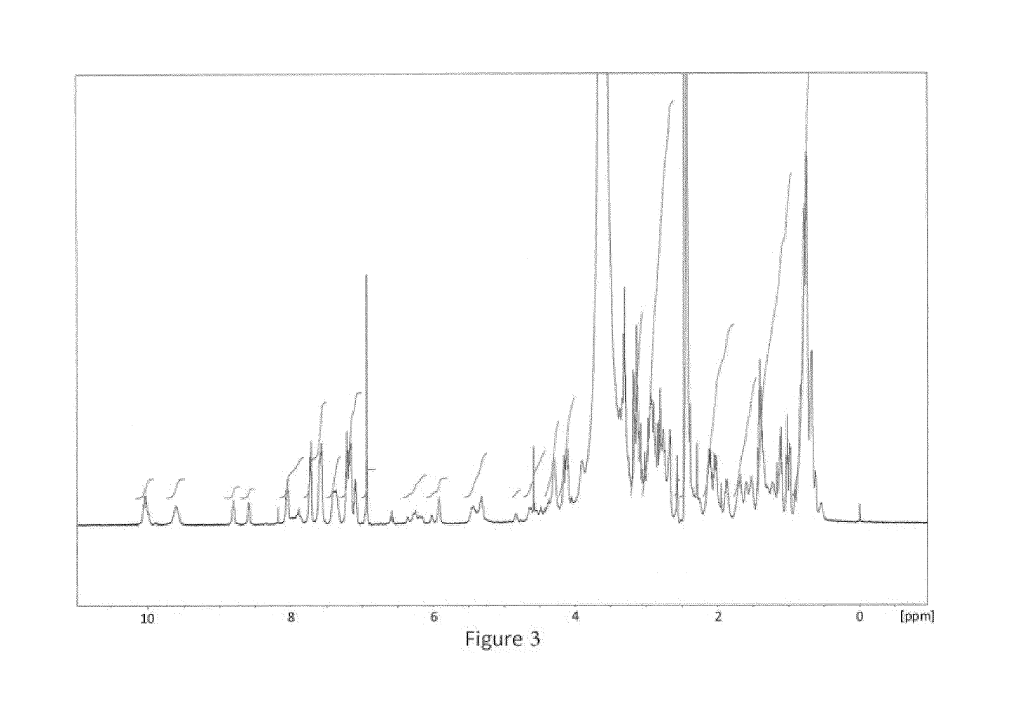

Tap-18Hr2 was synthesized with the formula shown below. FIG. 3 shows NMR spectrum of Tap-18Hr2.

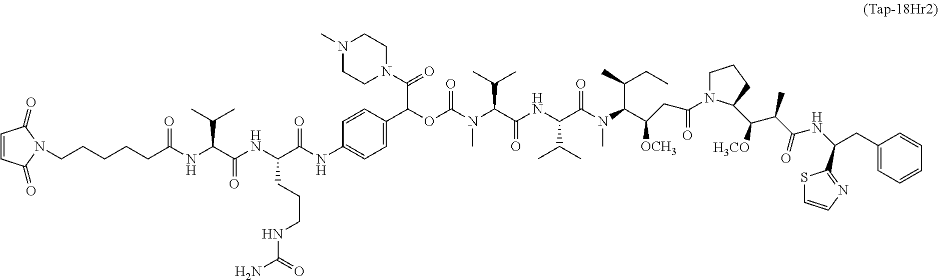 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C(=O)OC(C(=O)N1CCN(C)CC1)c1ccc(NC(=O)[C@H](CCCNC(N)=O)NC(=O)[C@@H](NC(=O)CCCCCN2C(=O)C=CC2=O)C(C)C)cc1)C(C)C]

Cell Lines

The human ovary cancer cells SKOV-3 (ATCC, Cat. No. HTB-77) were cultured in McCoy's 5A Medium (modified) (GIBCO, Cat. No. 16600) supplemented with 10% FBS (HyClone, Cat. No. SH30071.03), 100 U/mL penicillin/100 μg/mL streptomycin (GIBCO, Cat. No. 15140). The human breast cancer cells MDA-MB-453 (BCRC, Cat. No. 60429) were cultured in Leibovitz's L-15 medium (GIBCO, Cat. No. 11415) supplemented with 10% FBS (HyClone, Cat. No. SH30071.03), 100 U/mL penicillin/100 μg/mL streptomycin (GIBCO, Cat. No. 15140). The human breast cancer cells JIMT-1 (DSMZ, Cat. No. ACC 589) were cultured in Dulbecco's MEM medium (GIBCO, Cat. No. 11965) supplemented with 10% FBS (HyClone, Cat. No. SH30071.03), 100 U/mL penicillin/100 μg/mL streptomycin (GIBCO, Cat. No. 15140). The human gastric cancer cells NCI-N87 (CCRC, Cat. No. 60217) and the human T leukemia cells Jurkat (BCRC, Cat. No 60424) were cultured in RPMI Medium 1640 (GIBCO, Cat. No. 22400) supplemented with 10% FBS (HyClone, Cat. No. SH30071.03), 100 U/mL penicillin/100 μg/mL streptomycin (GIBCO, Cat. No. 15140).

Reagents

DTT and DTPA were obtained from Sigma-Aldrich (St. Louis, Mo.). TCEP was obtained from Acros (Morris Plains, N.J.). DTNB was obtained from Thermo Scientific (Rockford, Ill.). Sodium phosphate, sodium borate, and sodium chloride were obtained from J.T. Baker (Center Valley, Pa.). Cysteine was obtained from Alfa Aesar (Ward Hill, Mass.).

Generation of Anti-HER2-Cysteine Variants

Cysteine residue was introduced into humanized anti-HER2 antibody (Light chain as SEQ ID NO. 9 and heavy chain as SEQ ID NO.10 for IgG1 or SEQ ID NO. 11 for IgG4) with site-directed mutagenesis method. Briefly, mutagenesis was performed by overlapping PCR. Specific alternation in the desired base can be introduced by incorporating nucleotide changed primers. As the primers were extended, the mutation was created in the resulting amplicon. The mutation position and corresponding flanking sequence are listed in Table 3 below.

TABLE 3
Cysteine substituted mutants

| EU | | |
|---|---|---|
| numbering | | |
| (EU index in | | |
| Kabat) | Flanking Sequence | SEQ ID NO |
| Q147C (CL) | LLNNFYPREAKVCWKVDNALQSGNS | 34 |
| K188C (CL) | SSTLTLSKADYECHKVYACEVTHQG | 35 |
| G200C (CL) | KHKVYACEVTHQCLSSPVTKSFNRG | 36 |
| L201C(CL) | HKVYACEVTHQGCSSPVTKSFNRGE | 37 |
| V205C (CL) | EVTHQGLSSPCTKSFNRGEC | 38 |
| T206C (CL) | CEVTHQGLSSPVCKSFNRGEC | 39 |
| T155C(CH1) | GCLVKDYFPEPVCVSWNSGALTSGV | 40 |
| | (hIgG1~4) | |
| S157C(CH1) | LVKDYFPEPVTVCWNSGALTSGVHT | 41 |
| | (hIgG1~4) | |
| S165C(CH1) | PVTVSWNSGALTCGVHTFPAVLQSS | 42 |
| | (hIgG1~4) | |
| T169C(CH1) | SWNSGALTSGVHCFPAVLQSSGLYS | 43 |
| | (hIgG1~4) | |
| T197C(CH1) | VVTVPSSSLGTQCYICNVNHKPSNT | 44 |
| | (hIgG1) | |
| | VVTVPSSNFGTQCYTCNVDHKPSNT | 45 |
| | (hIgG2) | |
| | VVTVPSSSLGTQCYTCNVNHKPSNT | 46 |
| | (hIgG3) | |
| | VVTVPSSSLGTKCYTCNVDHKPSNT | 47 |
| | (hIgG4) | |
| I199C (CH1, | TVPSSSLGTQTYCCNVNHKPSNT | 48 |
| hIgG1) | (hIgG1) | |
| T199C (CH1, | TVPSSNFGTQTYCCNVDHKPSNT | 49 |
| hIgG2/3/4) | (hIgG2) | |
| | TVPSSSLGTQTYCCNVNHKPSNT | 50 |
| | (hIgG3) | |
| | TVPSSSLGTKTYCCNVDHKPSNT | 51 |
| | (hIgG4) | |
| T209C (CH1) | TYICNVNHKPSNCKVDKKVEPKSCD | 52 |
| | (hIgG1) | |
| | TYTCNVDHKPSNCKVDKTVERKCCV | 53 |
| | (hIgG2) | |
| | TYTCNVNHKPSNCKVDKRVELKTPL | 54 |
| | (hIgG3) | |
| | TYTCNVDHKPSNCKVDKRVESKYGP | 55 |
| | (hIgG4) | |
| V211C (CH1) | ICNVNHKPSNTKCDKKVEPKSCDKT | 56 |
| | (hIgG1) | |
| | TCNVDHKPSNTKCDKTVERKCCVEC | 57 |
| | (hIgG2) | |
| | TCNVNHKPSNTKCDKRVELKTPLGD | 58 |
| | (hIgG3) | |
| | TCNVDHKPSNTKCDKRVESKYGPPC | 59 |
| | (hIgG4) | |
| S442C(CH3) | EALHNHYTQKSLCLSPGK | 60 |
| | (hIgG1, hIgG2) | |
| | EALHNRFTQKSLCLSPGK | 61 |
| | (hIgG3) | |
| | EALHNHYTQKSLCLSLGK | 62 |
| | (hIgG4) | |

Production of Stable Cell Lines Expressing Anti-HER2-Cys Variants

Anti-HER2-Cysteine (Anti-HER2-Cys) variants (Table 1) were stably expressed and produced in Flp-In CHO cells (Invitrogen, Cat. No: R708-07). The DNA sequences of cysteine substituted antibody variants were inserted to pcDNA5/FRT vector (Invitrogen, Cat. No: V6010-20) and co-transfected with pOG44 (Invitrogen, Cat. No V6005-20) following the standard procedure provided by vendor. The culture supernatants of the established cell lines were collected and purified with protein A sepharose beads (GE Healthcare, Cat. No: 17-5280-04). The purified proteins were analyzed with both SDS-PAGE and size exclusion chromatography to ensure the quality of antibodies.

Conventional Conjugation of Anti-HER2-IgG1 Antibody

Anti-HER2-IgG1 (light chain as SEQ ID NO. 9, heavy chain as SEQ ID NO. 10) antibody was reduced with about 1.55 equivalents of TCEP in 0.025 M sodium borate pH 8, 0.025 M NaCl, 1 mM DTPA for 2 hours at 37° C. The protein concentration was quantified using an absorbance value of 1.48 at 280 nm for a 1.0 mg/mL solution, and the molar concentration determined using a molecular weight of 145,532 g/mol. The concentration of mAb-cysteine thiols produced was determined by titrating with DTNB. Typically 3.0 thiols/mAb was obtained. Partially reduced antibody was alkylated with 1.2 molar of maleiminocaproyl-drugs/mAb cysteine thiol or maleimide-drugs (Tap18Hr1, Tap-18Hr1)/mAb-cysteine thiol. The alkylation reaction was performed at 4° C. for 12˜16 hours. Cysteine (1 mM final) was used to quench any unreacted, excess maleimidocaproyl-drugs or maleimide-drugs. The Tap18Hr1 conjugation mixture was first diluted 5 fold with binding buffer, 10 mM sodium phosphate, 10 mM NaCl, 5% DMSO, pH 7.0, and applied to a hydroxyapatite column (Macroprep ceramic type I 40 μm, BioRad, Hercules, Calif.) at loading capacity of 1 mL hydroxyapatite per 20 mg of conjugated antibody named as Anti-HER2/Tap18Hr1. The column was previously equilibrated with 5 column volumes of binding buffer. Following sample application, the column was washed with 3 column volumes of binding buffer and then equilibrated with 5 column volumes of 10 mM sodium phosphate, 10 mM NaCl, pH 7.0. The binding ADC was then eluted with 200 mM sodium phosphate, 10 mM NaCl, pH 7.0. Following elution, the buffer was changed to Dulbecco's phosphate buffered saline using HiPrep™ 26/10 Desalting column (optional).

Site-Specific Conjugation of Anti-HER2-Cys Variants

To specifically conjugate linker payload Tap18Hr1 (Tap-18Hr1) on the introduced cysteine, a reducing/oxidation procedure was used. To remove cysteine or glutathione on the introduced cysteine site which could have occurred during culture condition, Anti-HER2-Cys variants were first treated with 10˜15 fold molar excess of TCEP (Acros Organics, Cat. No: 363830100) at 37° C. for 2-5 hours in PBS (Gibco®, Cat. No: 21600-069) containing 1 mM DTPA (Sigma-Aldrich, Cat. No: D6518). After removing the excess TCEP, the antibody was then re-oxidized with dehydroascorbic acid (DHA) (Sigma-Aldrich, Cat. No:261556) with 20˜70 fold molar excess over antibody at room temperature for 3˜5 hours or 4° C. for 3˜16 hours to ensure the re-formation of inter-chain disulfide bonds. The samples were buffer exchanged into PBS. The maleimide-linked drug payload (Tap18Hr1) was then added to react with free-thiols on the processed antibody. The excess payload was quenched with N-acetyl-L-cysteine (Sigma-Aldrich, Cat. No:A7250) and CHT ceramic hydroxyapatite (Bio-Rad, Cat. No:157-0040) were used to purify the conjugated antibody.

Drug Antibody Ratio (DAR) Determination by Reverse Phase HPLC Analysis

Prior to HPLC analysis, conjugate sample was treated with 6M guanidine hydrochloride and 20 mM DTT under 50° C. heating for 15 mins. 100 μg of the treated conjugate sample was applied to PLRP-S column (2.1×150 mm, 8 μm, 10001, Agilent). The flow rate was 0.8 mL/min and the column temperature was 80° C. Solvent A was 0.05% trifluoroacetic acid in mini Q water and solvent B was 0.04% trifluoroacetic acid in acetonitrile. The method consisted of the following: Isocratic 25% B for 3 ml, a 25 ml linear gradient to 50% B, a 2 ml linear gradient to 95% B, a 1 ml linear gradient to 25% B, and isocratic 25% B for 2 ml. Peak assignments were made with unconjugated antibody (L0 and H0). H1 and H2 were assigned by their elution time and UV spectra (the A248/280 ratio increases with drug loading).

Binding of Anti-HER2 Antibody and the Tap18Hr1 Conjugates to Cancer Cells

1×10⁵ cells were seeded per well in a v-bottomed 96-well plate and incubated with 100 μl of the unconjugated Abs or the ADCs at indicated concentrations. After 60˜90 minutes of incubation at 4° C., cells were washed once with 200 μl FACS buffer (lx PBS containing 1% FBS), stained with 100 μl of 1 μg/ml goat F(ab′)2-anti-human IgG (H+L)-RPE (Southern Biotech, Cat. No. 2043-09) in FACS buffer and then incubated at 4° C. for 30˜60 minutes. Cells were washed once with FACS buffer and analyzed by flow cytometry (BD LSR, BD Life Sciences).

In Vitro Cytotoxicity Assay: WST-1 Assay

SKOV-3 cells were seeded 5×10³ cells, MDA-MB-453 and JIMT-1 were seeded 2×10⁴ cells, NCI-N87 were seeded 4×10⁴, and Jurkat cells were seeded 2.5×10⁴ cells per well, on 96-well microtiter plates. Anti-HER2/Tap18Hr1 or unconjugated antibody were added in 6 replicates at the indicated concentrations in a final volume 200 μL/well. MDA-MB-453 cell were then incubated at 37° C. and 0% CO₂ for 96 hours, renew equivalent medium at 48 hours. SKOV-3, JIMT-1, NCI-N87, and Jurkat cells were incubated at 37° C. and 5% CO₂ for 68-72 hours. After incubation, the cell viability was detected by cell proliferation reagent WST-1 (Roche, Cat. No. 11644807001) following manufacturer's instructions. In brief, at the end of incubation 100 μL of medium was withdrawn and 10 μL/well of WST-1 was added. After optimal color development (when OD₄₅₀ of untreated control ≧1, absorbance at 450 nm (OD₄₅₀ value) was measured by spectrophotometer (Molecular Devices (Sunnyvale, Calif.), VERSAmax microplate reader). The mean of the replicates was obtained and background (medium control) was subtracted. The resultant OD₄₅₀ values were then used to calculate % inhibition according to the following formula: [OD₄₅₀ solvent−OD₄₅₀ sample]/[OD₄₅₀ solvent]*100.

### ADC Treatment in Cancer Xenograft Model

SKOV-3 Treated with Anti-HER2/Tap18Hr1

To establish a subcutaneous xenograft model, 1×10⁷ SKOV-3 cells in 100 μL of PBS containing 25% High Concentration Matrigel (BD Biosciences, Cat. No. 354248) were implanted into the right flank of 6-week-old female C.B-17 SCID mice (Lasco, Taipei, Taiwan). Anti-HER2/Tap18Hr1 was injected intravenously at 3 mg/kg in 100 μL approximately 2 hour after tumor cell inoculation (marked as Day 1). Tumor volume was measured once or twice weekly with a caliper in two perpendicular dimensions, and calculated according to the formula (0.52*length*width*width).

MDA-MB-453 Treated with Anti-HER2/Tap18Hr1

To establish a subcutaneous xenograft model, 1×10⁷ MDA-MB-453 cells in 150 μL of PBS containing 50% High Concentration Matrigel (BD Biosciences, Cat. No. 354248) were implanted into the right flank of 7-week-old female C.B-17 SCID mice (Lasco, Taipei, Taiwan). When average tumor volume reached 150 mm³, Anti-HER2/Tap18Hr1 was injected intravenously once at 3 mg/kg in 100 μL (marked as day 1). Tumor volume was measured twice weekly with a caliper in two perpendicular dimensions, and calculated according to the formula (0.52*length*width*width).

NCI-N87 Treated with Anti-HER2/Tap18Hr1 and Site-Specific Tap18Hr1-Conjugated Anti-HER2 Cysteine Variants

To establish a subcutaneous xenograft model, 5×10⁶ NCI-N87 cells in 100 μL of PBS were implanted into the right flank of 7-week-old female C.B-17 SCID mice (Lasco, Taipei, Taiwan). When average tumor volume reached 180 mm³, drug conjugated antibodies were injected intravenously (marked as day 1). Tumor volume was measured once or twice weekly with a caliper in two perpendicular dimensions, and calculated according to the formula (0.52*length*width*width).

JIMT-1 Treated with Anti-HER2/Tap18Hr1 and Site-Specific Tap18Hr1-Conjugated Anti-HER2 cysteine variants

To establish a subcutaneous xenograft model, 5×10⁶ JIMT-1 cells in 100 μL of PBS were implanted into the right flank of 6-week-old female C.B-17 SCID mice (Lasco, Taipei, Taiwan). When average tumor volume reached 100 mm³, drug conjugated antibodies were injected intravenously once at 3 mg/kg in 100 μL (marked as day 1). Tumor volume was measured twice weekly with a caliper in two perpendicular dimensions, and calculated according to the formula (0.52*length*width*width).

### Example 2

### In Vitro Cellular Binding Activity of Anti-HER2 Antibody Based Antibody Drug Conjugate (ADC) on Cancer Cells

### Anti-HER2/Tap18Hr1 Binding Ability

The binding ability of Anti-HER2 naked and Tap18Hr1-conjugated antibodies was evaluated in SKOV-3, NCI-N87, MDA-MB-453 and Jurkat cells. Data in Table 4 shows that the tested samples bind significantly to human HER2-expressing cell lines (NCI-N87, SKOV-3, and MDA-MB-453), but not to Jurkat which does not express human HER2. In addition, both naked and drug-conjugated antibodies bind to these cells with comparable mean fluorescence intensity (MFI). These results demonstrate that Anti-HER2/Tap18Hr1 retains antigen reactivity of naked antibody and binds to HER2-expressing cells effectively.

TABLE 4
Binding of Anti-HER2-IgG1 to cancer cells

| | Anti-HER2/Tap18Hr1 | Anti-HER2-IgG1 | Secondary |
|---|---|---|---|
| MFI | (3.3 μg/mL) | (3.3 μg/mL) | Ab only |
| NCI-N87 | 9810 | 9871 | 9 |
| SKOV-3 | 7595 | 8157 | 8 |
| MDA-MB-453 | 7085 | 8009 | 11 |
| Jurkat | 7 | 11 | 9 |

### The Binding Ability of Tap18Hr1-Conjugated Anti-HER2 Cysteine Variants in Breast JIMT-1, Gastric NCI-N87, and Ovarian SKOV-3 Cancer Cells

The binding ability of Anti-HER2-IgG1 Cys variants with or without drug conjugation was evaluated in breast JIMT-1 (Table 5-7), gastric NCI-N87 (Table 8), and ovarian SKOV-3 (Table 9) cancer cells. Data in Tables 5-9 shows that Anti-HER2-IgG1 cysteine variants bind comparably to all the tested cancer cells with Anti-HER2-IgG1 Ab. In addition, the site-specific conjugated Anti-HER2-IgG1 ADCs also retained antigen reactivity, except Anti-HER2-S442C-IgG1/Tap18Hr1 that displayed slightly lower affinity than other variants.

TABLE 5
Binding of Anti-HER2-IgG1 variants to JIMT-1 cells

| | Unconjugated Ab | Tap18Hr1 conjugates |
|---|---|---|
| MFI | (3.3 μg/mL) | (3.3 μg/mL) |
| Anti-HER2-S157C-IgG1 | 1555 | 1636 |
| Anti-HER2-S442C-IgG1 | 1402 | 1296 |
| Anti-HER2-IgG1 | 1574 | 1615 |
| 2^{nd} Ab only | 9 | *ND |

*ND: Not Determined.

TABLE 6
Binding of Anti-HER2-IgG1 variants to JIMT-1 cells

| | Unconjugated Ab | Tap18Hr1 conjugates |
|---|---|---|
| MFI | (3.3 μg/mL) | (3.3 μg/mL) |
| Anti-HER2-T155C-IgG1 | 1448 | 1492 |
| Anti-HER2-T169C-IgG1 | 1554 | 1513 |
| Anti-HER2-IgG1 | 1550 | *ND |
| 2^{nd} Ab only | 5 | *ND |

*ND: Not Determined.

TABLE 7
Binding of Anti-HER2-IgG1 variants to breast JIMT-1 cancer cells

| | Unconjugated Ab | Tap18Hr1 conjugates |
|---|---|---|
| MFI | (3.3 μg/mL) | (3.3 μg/mL) |
| Anti-HER2-T209C-IgG1 | 1737 | 1796 |
| Anti-HER2-Q147C-IgG1 | 1772 | 1897 |
| Anti-HER2-G200C-IgG1 | 1924 | 1944 |
| Anti-HER2-L201C-IgG1 | 1807 | 1935 |
| Anti-HER2-T206C-IgG1 | 1765 | 1841 |
| Anti-HER2-IgG1 | 1738 | 1906 |
| Isotype control | 4 | *ND |
| 2^{nd} Ab only | 4 | *ND |

*ND: Not Determined.

TABLE 8
Binding of Anti-HER2-IgG1 variants
to gastric NCI-N87 cancer cells

| | Unconjugated Ab | Tap18Hr1 conjugates |
|---|---|---|
| MFI | (3.3 μg/mL) | (3.3 μg/mL) |
| Anti-HER2-S157C-IgG1 | 9667 | 9587 |
| Anti-HER2-S442C-IgG1 | 9645 | 9417 |
| Anti-HER2-T209C-IgG1 | 9629 | 9601 |
| Anti-HER2-Q147C-IgG1 | 9696 | 9627 |
| Anti-HER2-G200C-IgG1 | 9731 | 9639 |
| Anti-HER2-L201C-IgG1 | 9664 | 9669 |
| Anti-HER2-T206C-IgG1 | 9691 | 9633 |
| Anti-HER2-IgG1 | 9698 | 9628 |
| Isotype control | 4 | *ND |
| 2^{nd} Ab only | 7 | *ND |

*ND: Not Determined.

TABLE 9
Binding of Anti-HER2-IgG1 variants
to ovarian SKOV-3 cancer cells

| | Unconjugated Ab | Tap18Hr1 conjugates |
|---|---|---|
| MFI | (3.3 μg/mL) | (3.3 μg/mL) |
| Anti-HER2-S157C-IgG1 | 6194 | 6427 |
| Anti-HER2-S442C-IgG1 | 6177 | 5636 |
| Anti-HER2-T209C-IgG1 | 6232 | 6232 |
| Anti-HER2-Q147C-IgG1 | 6295 | 6408 |
| Anti-HER2-G200C-IgG1 | 6826 | 6412 |
| Anti-HER2-L201C-IgG1 | 6491 | 6552 |
| Anti-HER2-T206C-IgG1 | 6321 | 6440 |
| Anti-HER2-IgG1 | 6206 | 6287 |
| Isotype control | 3 | *ND |
| 2^{nd} Ab only | 3 | *ND |

*ND: Not Determined.

The binding ability of Anti-HER2-IgG4p Cys variants with or without drug conjugation was evaluated in breast JIMT-1 (Table 10-11), gastric NCI-N87 (Table 12), and ovarian SKOV-3 (Table 13) cancer cells. Data in Table 10-13 shows that binding of Anti-HER2-IgG4p cysteine variants is comparable to that of Anti-HER2-IgG4p antibody, yet lower when compared with that of Anti-HER2-IgG1 antibody (Table 5-9), indicating that the decreased binding activity was attributed to IgG4 isotype rather than cysteine mutation. Overall, the site-specific conjugated Anti-HER2-IgG4p ADCs retained antigen reactivity. Similar to IgG1 variants, Anti-HER2-S442C-IgG4p also displayed slightly lower affinity than other variants.

TABLE 10
Binding of Anti-HER2-IgG4p cysteine variants to JIMT-1 cells

| | Unconjugated Ab | Tap18Hr1 conjugates |
|---|---|---|
| MFI | (3.3 μg/mL) | (3.3 μg/mL) |
| Anti-HER2-T155C-IgG4p | 568 | 574 |
| Anti-HER2-T169C-IgG4p | 527 | 543 |
| Anti-HER2-S442C-IgG4p | 556 | 405 |
| Anti-HER2-IgG4p | 543 | *ND |
| Anti-HER2-IgG1 | 1026 | 797 |
| Isotype control (10 μg/ml) | 3.79 | *ND |
| 2^{nd} Ab only | 3.29 | *ND |

*ND: Not Determined.

TABLE 11
Binding of Anti-HER2-IgG1, Anti-HER2-IgG4p, and Anti-HER2-
IgG4p cysteine variants to breast JIMT-1 cancer cells

| | Unconjugated Ab | Tap18Hr1 conjugates |
|---|---|---|
| MFI | (3.3 μg/mL) | (3.3 μg/mL) |
| Anti-HER2-T199C-IgG4p | 1020 | 991 |
| Anti-HER2-T209C-IgG4p | 1001 | 1025 |
| Anti-HER2-V211C-IgG4p | 1040 | 1026 |
| Anti-HER2-K188C-IgG4p | 1057 | 1075 |
| Anti-HER2-IgG4p | *ND | *ND |
| Anti-HER2-IgG1 | 1738 | 1906 |
| Isotype control (10 μg/ml) | 4 | *ND |
| 2^{nd} Ab only | 4 | *ND |

*ND: Not Determined.

TABLE 12
Binding of Anti-HER2-IgG1, Anti-HER2-IgG4p, and Anti-HER2-
IgG4p cysteine variants to gastric NCI-N87 cancer cells

| | Unconjugated Ab | Tap18Hr1 conjugates |
|---|---|---|
| MFI | (3.3 μg/mL) | (3.3 μg/mL) |
| Anti-HER2-T155C-IgG4p | 8562 | 8510 |
| Anti-HER2-T169C-IgG4p | 8704 | 8475 |
| Anti-HER2-S442C-IgG4p | 8433 | 8385 |
| Anti-HER2-T199C-IgG4p | 8524 | 8347 |
| Anti-HER2-T209C-IgG4p | 8331 | 8366 |
| Anti-HER2-V211C-IgG4p | 8429 | 8388 |
| Anti-HER2-K188C-IgG4p | 8562 | 8501 |
| Anti-HER2-IgG4p | 8794 | *ND |
| Anti-HER2-IgG1 | 9698 | 9628 |
| Isotype control (10 μg/ml) | 4 | *ND |
| 2^{nd} Ab only | 7 | *ND |

*ND: Not Determined.

TABLE 13
Binding of Anti-HER2-IgG1, Anti-HER2-IgG4p, and Anti-HER2-
IgG4p cysteine variants to ovarian SKOV-3 cancer cells

| | Unconjugated Ab | Tap18Hr1 conjugates |
|---|---|---|
| MFI | (3.3 μg/mL) | (3.3 μg/mL) |
| Anti-HER2-T155C-IgG4p | 4619 | 4941 |
| Anti-HER2-T169C-IgG4p | 4579 | 4966 |
| Anti-HER2-S442C-IgG4p | 4480 | 4362 |
| Anti-HER2-T199C-IgG4p | 5001 | 4625 |
| Anti-HER2-T209C-IgG4p | 4430 | 4582 |
| Anti-HER2-V211C-IgG4p | 4525 | 4659 |
| Anti-HER2-K188C-IgG4p | 4613 | 4641 |
| Anti-HER2-IgG4p | 4607 | *ND |
| Anti-HER2-IgG1 | 6206 | 6287 |
| Isotype control (10 μg/ml) | 3 | *ND |
| 2^{nd} Ab only | 3 | *ND |

*ND: Not Determined.

### Example 3

### In Vitro Cytotoxicity Effects of Anti-HER2 Antibody Based Antibody Drug Conjugate (ADC) on Cancer Cells

The in vitro cytotoxic activity of conventional conjugated Anti-HER2 antibody (Anti-HER2/Tap18Hr1) was evaluated in the HER2 positive cancer cell lines (NCI-N87, SKOV-3, MDA-MB-453, and JIMT-1) and a HER2 negative cell line (Jurkat). Cytotoxicity by the naked antibody was also tested in parallel. At 5 and 1.25 μg/mL, although anti-HER2 naked antibody can induce cytotoxicity in NCI-N87 (gastric cancer cells) and MBA-MD-453 (breast cancer cells), conventional conjugated Anti-HER2 was even more potent (Table 14). For JIMT-1 (breast cancer cells) and SKOV-3 (ovarian cancer cells), only drug conjugated Anti-HER2 can cause more than 50% growth inhibition at 5 and 1.25 μg/mL (Table 14 & 15). No toxicity was observed in the HER2 negative cell line Jurkat. These results demonstrate that Tap18Hr1 conjugated Anti-HER2 delivered cytotoxic drug to the target cancer cells with antigen specificity.

TABLE 14
In vitro cytotoxic activity

| | | |
|---|---|---|
| | 5 | 1.25 |
| (% inhibition) | μg/mL | μg/mL |

| | | | |
|---|---|---|---|
| NCI-N87 | Anti-HER2/Tap18Hr1 | 68.5 | 69.7 |
| | Anti-HER2 | 46.7 | 36.9 |
| SKOV-3 | Anti-HER2/Tap18Hr1 | 57.3 | 54.8 |
| | Anti-HER2 | 0.9 | −2.9 |
| MDA-MB-453 | Anti-HER2/Tap18Hr1 | 82.3 | 80.9 |
| | Anti-HER2 | 58.9 | 56.8 |
| Jurkat | Anti-HER2/Tap18Hr1 | 1.1 | −2.7 |
| | Anti-HER2 | 2.3 | −1.4 |

Negative values indicate no cytotoxicity detected.

TABLE 15
In vitro cytotoxic activity

| | | |
|---|---|---|
| | 5 | 1.25 |
| (% inhibition) | μg/mL | μg/mL |

| | | | |
|---|---|---|---|
| JIMT-1 | Anti-HER2/Tap18Hr1 | 63.5 | 58.5 |
| | Anti-HER2 | 9.3 | 7.2 |

The in vitro cytotoxic activity of the site-specific conjugated Anti-HER2-Cys variants was also evaluated in NCI-N87, JIMT-1, and SKOV-3 cells. Table 16 & 17 show the cytotoxic assay result of tested ADCs of Anti-HER2 cysteine variants. At 5 and 1.25 μg/mL, the site-specific ADCs were potent in killing the HER2 positive carcinoma cells (NCI-N87, JIMT-1, and SKOV-3) but not in the HER2 negative cell line (Jurkat). Despite slightly lower binding, Anti-HER2-S442C/Tap18Hr1 induced similar degree of cytotoxicity in antigen expressing cells as other cysteine variants. These results demonstrate that the site-specific Anti-HER2 ADCs can deliver cytotoxic drug to the target cancer cells with antigen specificity.

TABLE 16
In vitro cytotoxic activity by the Tap18Hr1
conjugated Anti-HER2-Cys variants

| | | 5 | 1.25 |
|---|---|---|---|
| (% inhibition) | | μg/mL | μg/mL |
| NCI-N87 | Anti-HER2-S157C-IgG1/Tap18Hr1 | 74.55 | 73.15 |
| | Anti-HER2-S442C-IgG1/Tap18Hr1 | 74.17 | 74.75 |
| | Anti-HER2-T155C-IgG4p/Tap18Hr1 | 74.89 | 74.21 |
| | Anti-HER2-T169C-IgG4p/Tap18Hr1 | 73.63 | 72.63 |
| | Anti-HER2-S442C-IgG4p/Tap18Hr1 | 74.91 | 73.83 |
| JIMT-1 | Anti-HER2-S157C-IgG1/Tap18Hr1 | 79.54 | 75.76 |
| | Anti-HER2-S442C-IgG1/Tap18Hr1 | 77.90 | 75.11 |
| | Anti-HER2-T155C-IgG4p/Tap18Hr1 | 76.35 | 72.95 |
| | Anti-HER2-T169C-IgG4p/Tap18Hr1 | 76.05 | 71.31 |
| | Anti-HER2-S442C-IgG4p/Tap18Hr1 | 74.67 | 72.15 |
| Jurkat | Anti-HER2-S157C-IgG1/Tap18Hr1 | 11.02 | 10.55 |
| | Anti-HER2-S442C-IgG1/Tap18Hr1 | 6.51 | 9.16 |
| | Anti-HER2-T155C-IgG4p/Tap18Hr1 | −1.32 | 1.68 |
| | Anti-HER2-T169C-IgG4p/Tap18Hr1 | −1.84 | −0.13 |
| | Anti-HER2-S442C-IgG4p/Tap18Hr1 | −7.85 | −8.46 |

TABLE 17
In vitro cytotoxic activity by the Tap18Hr1
conjugated Anti-HER2-Cys variants

| | | 5 | 1.25 |
|---|---|---|---|
| (% inhibition) | | μg/mL | μg/mL |
| NCI-N87 | Anti-HER2-T209C-IgG1/Tap18Hr1 | 70.33 | 69.80 |
| | Anti-HER2-T199C-IgG4p/Tap18Hr1 | 69.95 | 69.46 |
| | Anti-HER2-T209C-IgG4p/Tap18Hr1 | 71.53 | 71.72 |
| | Anti-HER2-V211C-IgG4p/Tap18Hr1 | 72.65 | 71.25 |
| | Anti-HER2-K188C-IgG4p/Tap18Hr1 | 70.88 | 71.06 |
| JIMT-1 | Anti-HER2-T209C-IgG1/Tap18Hr1 | 54.65 | 49.31 |
| | Anti-HER2-T199C-IgG4p/Tap18Hr1 | 69.07 | 56.87 |
| | Anti-HER2-T209C-IgG4p/Tap18Hr1 | 59.21 | 49.53 |
| | Anti-HER2-V211C-IgG4p/Tap18Hr1 | 59.97 | 50.87 |
| | Anti-HER2-K188C-IgG4p/Tap18Hr1 | 58.91 | 51.01 |
| SKOV-3 | Anti-HER2-S157C-IgG1/Tap18Hr1 | 63.80 | 56.95 |
| | Anti-HER2-S442C-IgG1/Tap18Hr1 | 70.00 | 60.64 |
| | Anti-HER2-T209C-IgG1/Tap18Hr1 | 67.58 | 63.90 |
| | Anti-HER2-T155C-IgG4p/Tap18Hr1 | 63.91 | 61.22 |
| | Anti-HER2-T169C-IgG4p/Tap18Hr1 | 64.84 | 61.88 |
| | Anti-HER2-T199C-IgG4p/Tap18Hr1 | 72.32 | 63.52 |
| | Anti-HER2-T209C-IgG4p/Tap18Hr1 | 68.65 | 64.39 |
| | Anti-HER2-V211C-IgG4p/Tap18Hr1 | 66.26 | 60.77 |
| | Anti-HER2-K188C-IgG4p/Tap18Hr1 | 66.63 | 63.03 |

### Example 4

### SKOV-3 Xenograft Treated with Conventional Conjugated Anti-HER2

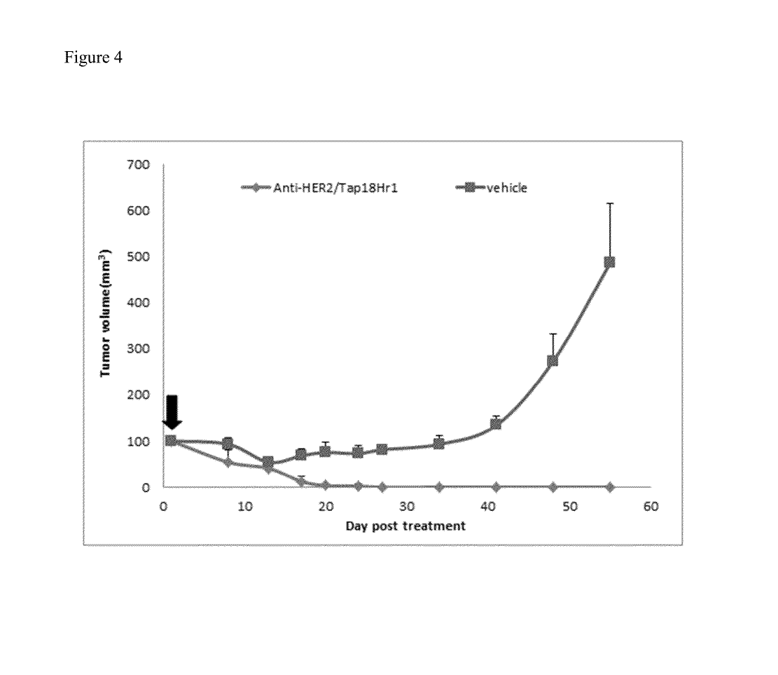

The efficacy of Anti-HER2/Tap18Hr1 was evaluated in vivo against the ovarian cancer cells SKOV-3. Mice were treated intravenously with vehicle (PBS, 100 μL) or a single dose of ADC at 3 mg/kg in 100 μL approximately two hour after tumor cell inoculation (marked as Day 1). The tumor size on Day 1 was recorded as 100 mm³ due to the inoculation volume including Matrigel. Injected matrigel were absorbed by Day 15, while the tumor was established and grew steadily in vehicle group (FIG. 4). Treatment with Anti-HER2/Tap18Hr1 suppressed tumor growth at Day 17, and all mice (5/5) in this group showed undetectable tumor since Day 27. No toxicity was observed as body weight of both groups gained steadily. The data show that with a single injection, Anti-HER2/Tap18Hr1 can effectively inhibit growth of antigen positive tumor grafted in SCID mice.

### Example 5

### MDA-MB-453 Xenograft Treated with Conventional Conjugated Anti-HER2

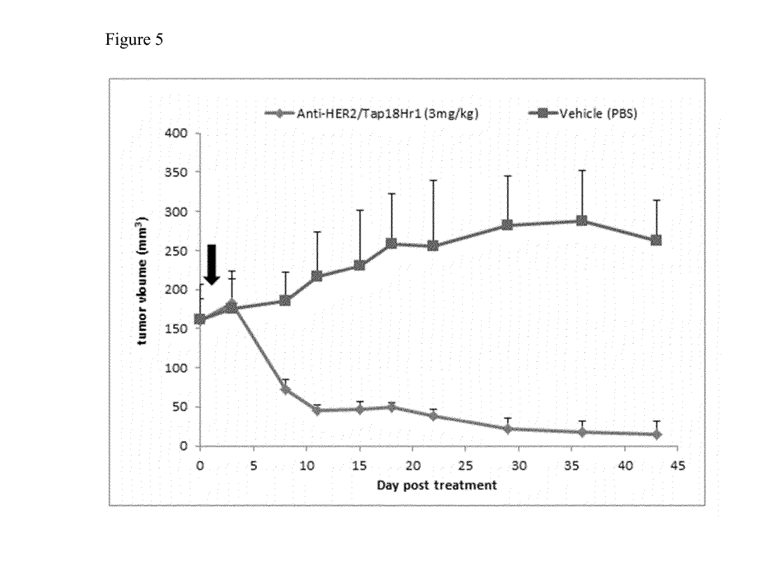

The efficacy of Anti-HER2/Tap18Hr1 was evaluated in vivo against the breast cancer cells MDA-MB-453. When the average inoculated tumor size reached ˜150 mm³, mice were treated intravenously with PBS (vehicle, 100 μL) or a single dose of ADC at 3 mg/kg in 100 μL (marked as Day 1). The Anti-HER2/Tap18Hr1 group showed tumor regressed at Day 8, mean tumor size was further suppressed down to <50 mm³ since Day 11 (FIG. 5). At the end of study, 3 out of 6 mice showed complete tumor regression. The body weight of mice gained steadily in both groups. The data show that Anti-HER2/Tap18Hr1 can effectively inhibit growth of antigen positive tumor grafted in SCID mice.

### Example 6

### NCI-N87 Xenograft Treated with Tap18Hr1 Conventional Conjugated Anti-HER2 and Site-Specific Conjugated Anti-HER2-Cys Variants

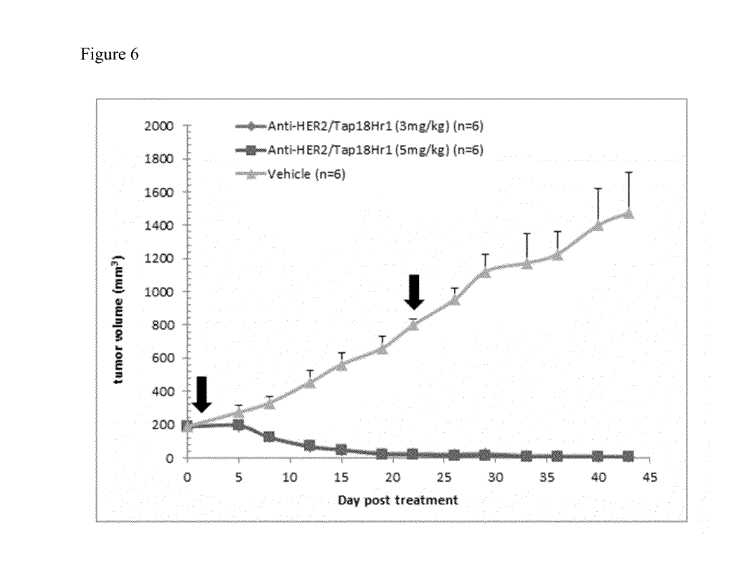

The efficacy of conventional conjugated Anti-HER2/Tap18Hr1 was evaluated in vivo against the gastric cancer cells NCI-N87. When the average tumor size reached ˜180 mm³, mice were treated intravenously with PBS (vehicle in 100 μL) or ADC (3 or 5 mg/kg in 100 μL) twice (marked as Day 1 and Day 22). While tumor of the vehicle group grew and approached 500 mm³ at day 15 (FIG. 6), Anti-HER2/Tap18Hr1 group showed delayed tumor growth at Day 5, mean tumor size was further suppressed down to <30 mm³ since Day 19. At the end of study, most of tumors were under 10 mm³ in both ADC treated groups. The body weight of mice gained steadily in three groups. The data show that Anti-HER2/Tap18Hr1 can effectively inhibit growth of antigen positive tumor grafted in SCID mice.

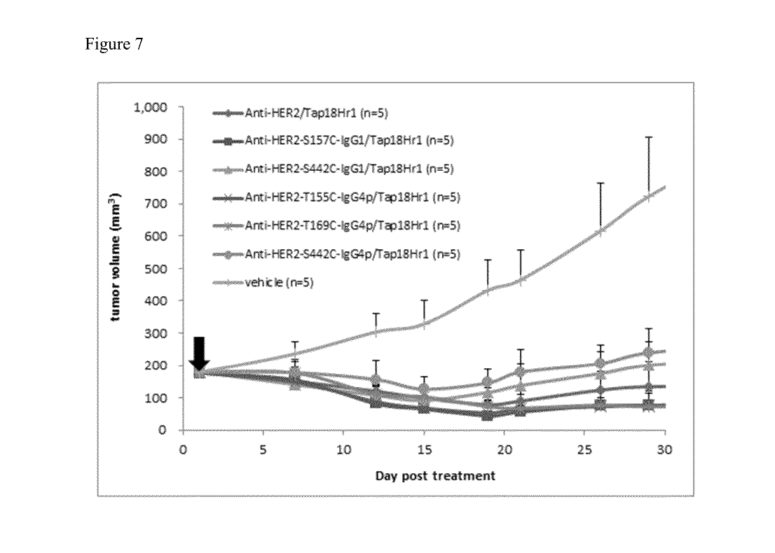

The efficacy of site-specific conjugated Anti-HER2-Cys variants was evaluated in vivo against the gastric cancer cells NCI-N87. When the average of tumor size reached ˜180 mm³, mice were treated intravenously with PBS (vehicle, 100 μL) or ADCs with equivalent drug dose (9.7 m/kg Tap18Hr1 in 100 μL) (marked as Day 1). As shown in FIG. 7, all site-specific conjugated variants treated mice showed significantly delayed tumor growth compared to vehicle group. Body weight remained unchanged in ADC-treated group and slightly increased in vehicle group due to the weight of tumor. The data demonstrate that with a single injection, site-specific conjugated Anti-HER2-Cys variants can effectively inhibit growth of antigen positive tumor grafted in SCID mice.

### Example 7

### JIMT-1 Xenograft Treated with Tap18Hr1 Conventional Conjugated Anti-HER2 and Site-Specific Conjugated Anti-HER2-Cys Variants

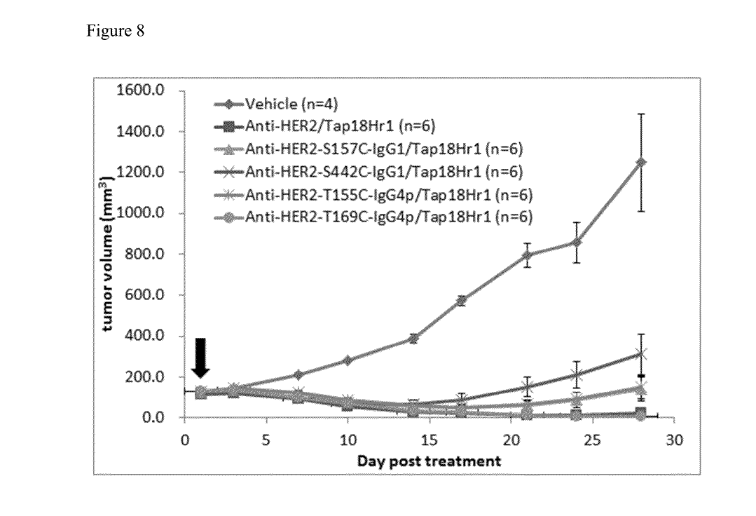

The efficacy of conventional conjugated Anti-HER2/Tap18Hr1 and site-specific conjugated Anti-HER2-Cys variants were evaluated in vivo against a known Herceptin-resistant breast cancer cells JIMT-1. When the average tumor size reached ˜100 mm³, mice were treated once intravenously with PBS (vehicle control, 100 μL) or ADC (3 mg/kg in 100 μL) (marked as Day 1). As shown in FIG. 8, all the ADC treatment groups showed significantly delayed tumor growth since Day 7 compared to the vehicle group. The body weight of mice gained steadily in all the groups. The data demonstrated that with a single dosing, not only conventional Anti-HER2/Tap18Hr1 but also site-specific conjugated Anti-HER2-Cys variants can effectively inhibit growth of HER2 positive tumor grafted in SCID mice.

What is claimed is:

1. A compound of formula (I): 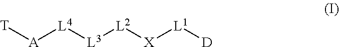
or a salt or solvate or stereoisomer thereof;
wherein:
D is a drug moiety;
T is a targeting moiety wherein T is an antibody that binds specifically to a human HER2;
X is a hydrophilic self-immolative linker;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit.

2. The compound of claim 1, wherein the compound is of formula (II): 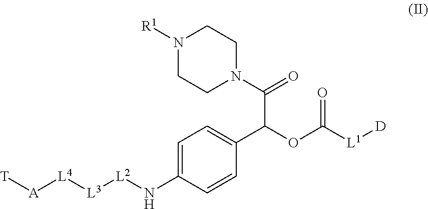
R¹ is hydrogen, unsubstituted or substituted C₁₋₃ alkyl, or unsubstituted or substituted heterocyclyl.

3. A compound of formula (Ia): 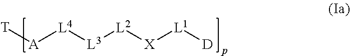
or a salt or solvate or stereoisomer thereof;
wherein:
p is 1, 2, 3, 4, 5, 6, 7, 8, 9, 10, 11, 12, 13, 14, 15, 16, 17, 18, 19, or 20;
D is a drug moiety;
T is a targeting moiety wherein T is an antibody that binds specifically to a human HER2;
X is a hydrophilic self-immolative linker;
L¹ is a bond, a self-immolative linker, or a cyclization self-elimination linker;
L² is a bond or a self-immolative linker;
wherein if L¹ is a self-immolative linker or a cyclization self-elimination linker, then L² is a bond;
wherein if L² is a self-immolative linker, then L¹ is a bond;
L³ is a peptide linker;
L⁴ is a bond or a spacer; and
A is an acyl unit.

4. The compound of claim 3, wherein the compound is of formula (IIa): 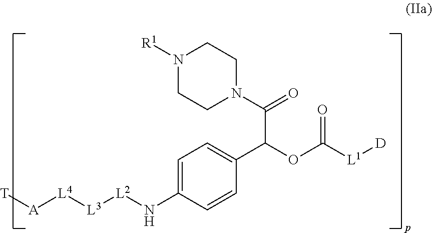
R¹ is hydrogen, unsubstituted or substituted C₁₋₃ alkyl, or unsubstituted or substituted heterocyclyl.

5. The compound of claim 3, wherein p is 1, 2, 3, or 4.

6. The compound of claim 3, wherein D is an amino group-containing drug moiety, wherein the drug is connected to L¹ or X through the amino group of the amino group-containing drug moiety.

7. The compound of claim 6, wherein D is duocarmycin, dolastatin, tubulysin, doxorubicin (DOX), paclitaxel, or mitomycin C (MMC), or an amino derivative thereof.

8. (canceled)

9. The compound of claim 3, wherein L¹ is a bond.

10. The compound of claim 3, wherein L¹ is a self-immolative linker or a cyclization self-elimination linker.

11. The compound of claim 10, wherein L¹ is an aminobenzyloxycarbonyl linker.

12. The compound of claim 10, wherein L¹ is selected from the group consisting of 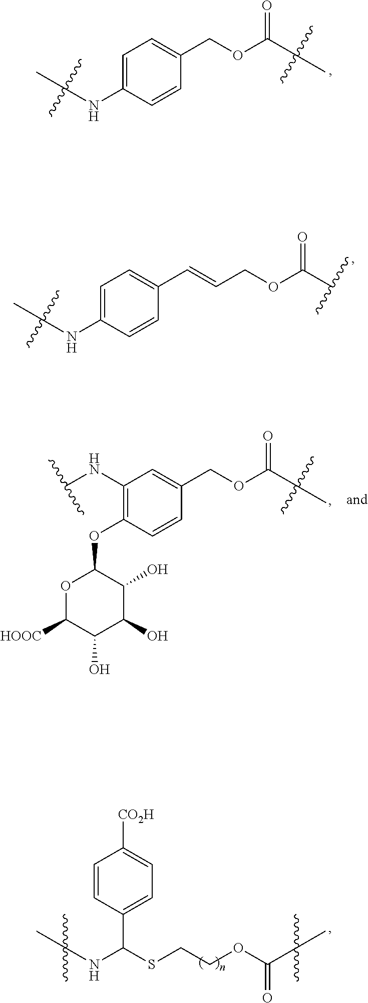
wherein n is 1 or 2.

13. The compound of claim 10, wherein L¹ is selected from the group consisting of

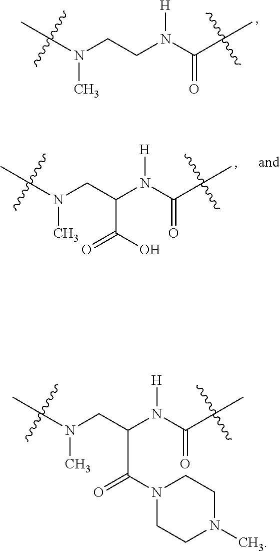

14. The compound of claim 3, wherein L² is a bond.

15. The compound of claim 9, wherein L² is a self-immolative linker.

16. The compound of claim 15, wherein L² is an aminobenzyloxycarbonyl linker.

17. The compound of claim 15, wherein L² is selected from 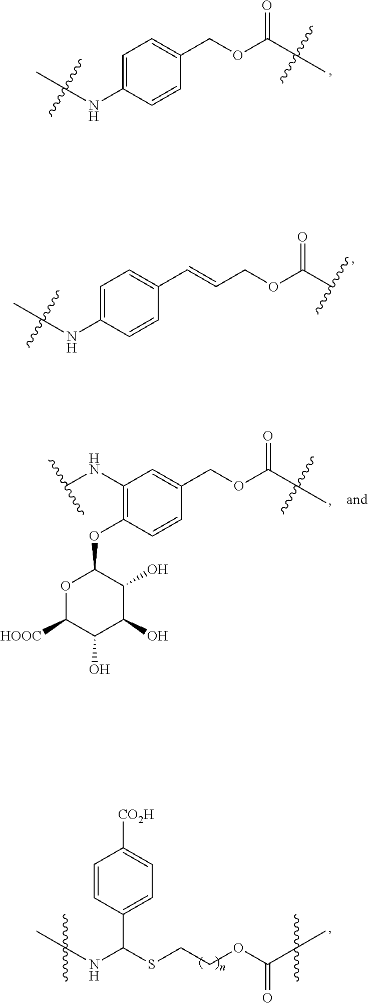
wherein n is 1 or 2.

18. The compound of claim 3, wherein L³ is a peptide linker of 1 to 10 amino acid residues.

19. The compound of claim 18, wherein L³ is a peptide linker of 2, 3, or 4 amino acid residues.

20. The compound of claim 3, wherein L³ is a peptide linker comprising at least one lysine or at least one arginine residue.

21. The compound of claim 3, wherein L³ is a peptide linker comprising an amino acid residue selected from lysine, D-lysine, citrulline, arginine, proline, histidine, ornithine and glutamine.

22. The compound of claim 3, wherein L³ is a peptide linker comprising an amino acid residue selected from valine, isoleucine, phenylalanine, methionine, asparagine, proline, alanine, leucine, tryptophan, and tyrosine.

23. The compound of claim 18, wherein L³ is a dipeptide unit selected from valine-citrulline, proline-lysine, methionine-D-lysine, asparagine-D-lysine, isoleucine-proline, phenylalanine-lysine, and valine-lysine.

24. The compound of claim 23, wherein L³ is valine-citrulline.

25. The compound of claim 3, wherein L⁴ is a bond.

26. The compound of claim 3, wherein L⁴ is a spacer.

27. The compound of claim 26, wherein the spacer is polyalkylene glycol, alkylene, alkenylene, alkynylene, or polyamine.

28. The compound of claim 26, wherein L⁴ is L^{4a}-C(O), L^{4a}-C(O)—NH, L^{4a}-S(O)₂, or L^{4a}-S(O)₂—NH, wherein each L^{4a} is independently polyalkylene glycol, alkylene, alkenylene, alkynylene, or polyamine.

29. The compound of claim 26, wherein L⁴ is L^{4a}-C(O), wherein L^{4a} is polyalkylene glycol, alkylene, alkenylene, alkynylene, or polyamine.

30. The compound of claim 26, wherein L⁴ is L^{4a}-C(O), wherein L^{4a} is a polyalkylene glycol.

31. The compound of claim 26, wherein L⁴ is L^{4a}-C(O), wherein L^{4a} is a polyethylene glycol.

32. The compound of claim 26, wherein the spacer is of the formula —CH₂—(CH₂—O—CH₂)ₘ—CH₂—C(O)—, wherein m is the integer 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

33. The compound of claim 26, wherein L⁴ is L^{4a}-C(O), wherein L^{4a} is alkylene.

34. The compound of claim 3, wherein A is selected from the group consisting of 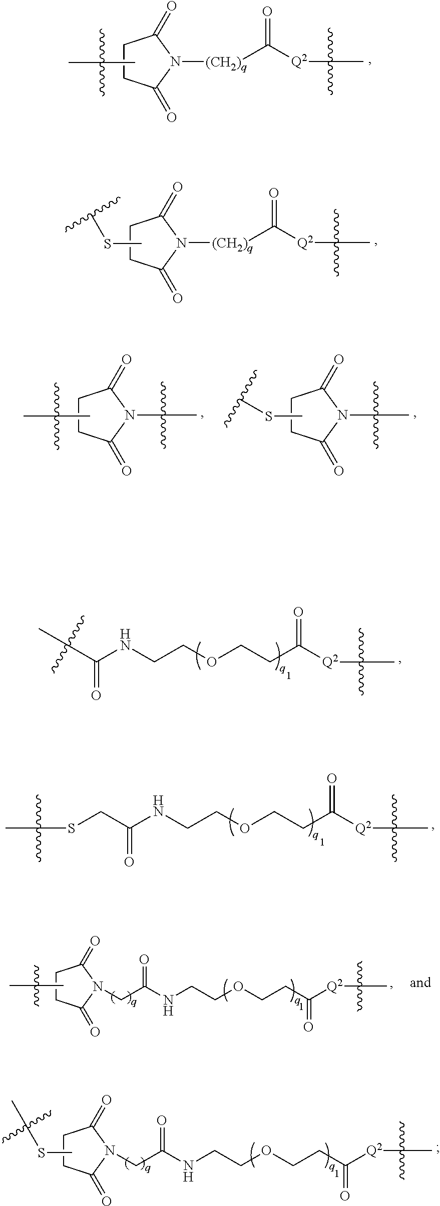
wherein each Q² is NH or O, each q is independently an integer from 1 to 10, and each q₁ is independently an integer from 1 to 10.

35. The compound of claim 34, wherein A is 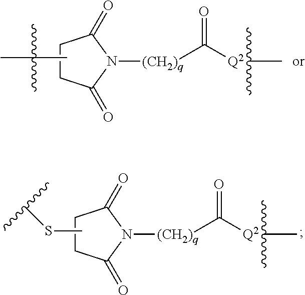
wherein each Q2 is independently NH or O and each q is independently the integer 1, 2, 3, 4, 5, 6, 7, 8, 9, or 10.

36. The compound of claim 35, wherein q is 2, 3, 4, or 5.

37. The compound of claim 3, wherein A is selected from the group 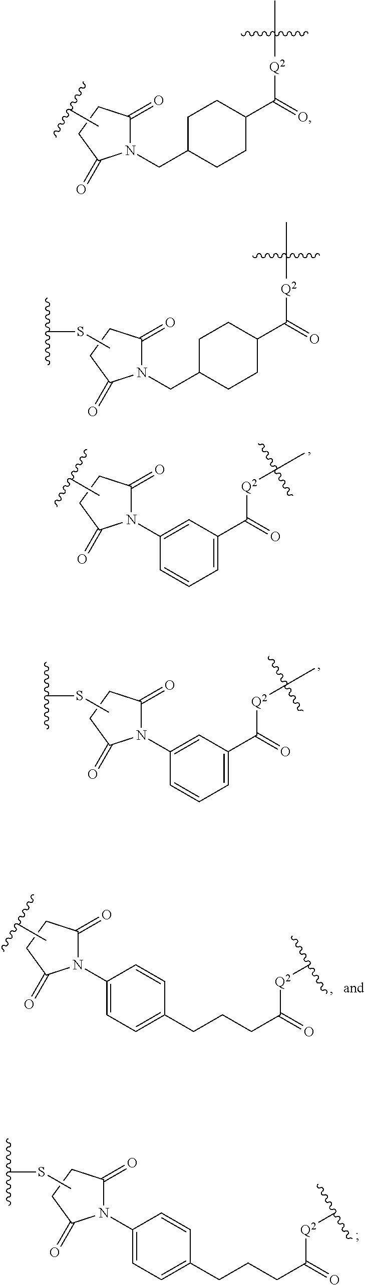
wherein each Q² is independently NH or O.

38. (canceled)

39. The compound of claim 6, wherein D is selected from the group consisting of:

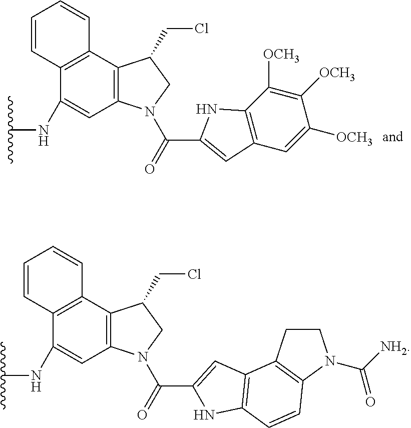 [SMILES: *Nc1cc2c(c3ccccc13)[C@H](CCl)CN2C(=O)c1cc2cc(OC)c(OC)c(OC)c2[nH]1 ; *Nc1cc2c(c3ccccc13)[C@H](CCl)CN2C(=O)c1cc2c3c(ccc2[nH]1)N(C(N)=O)CC3]

40. The compound of claim 6, wherein D is:

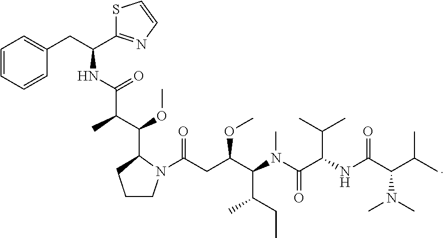 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C)C(C)C]

41. The compound of claim 3, wherein A-L⁴-L³-L² is

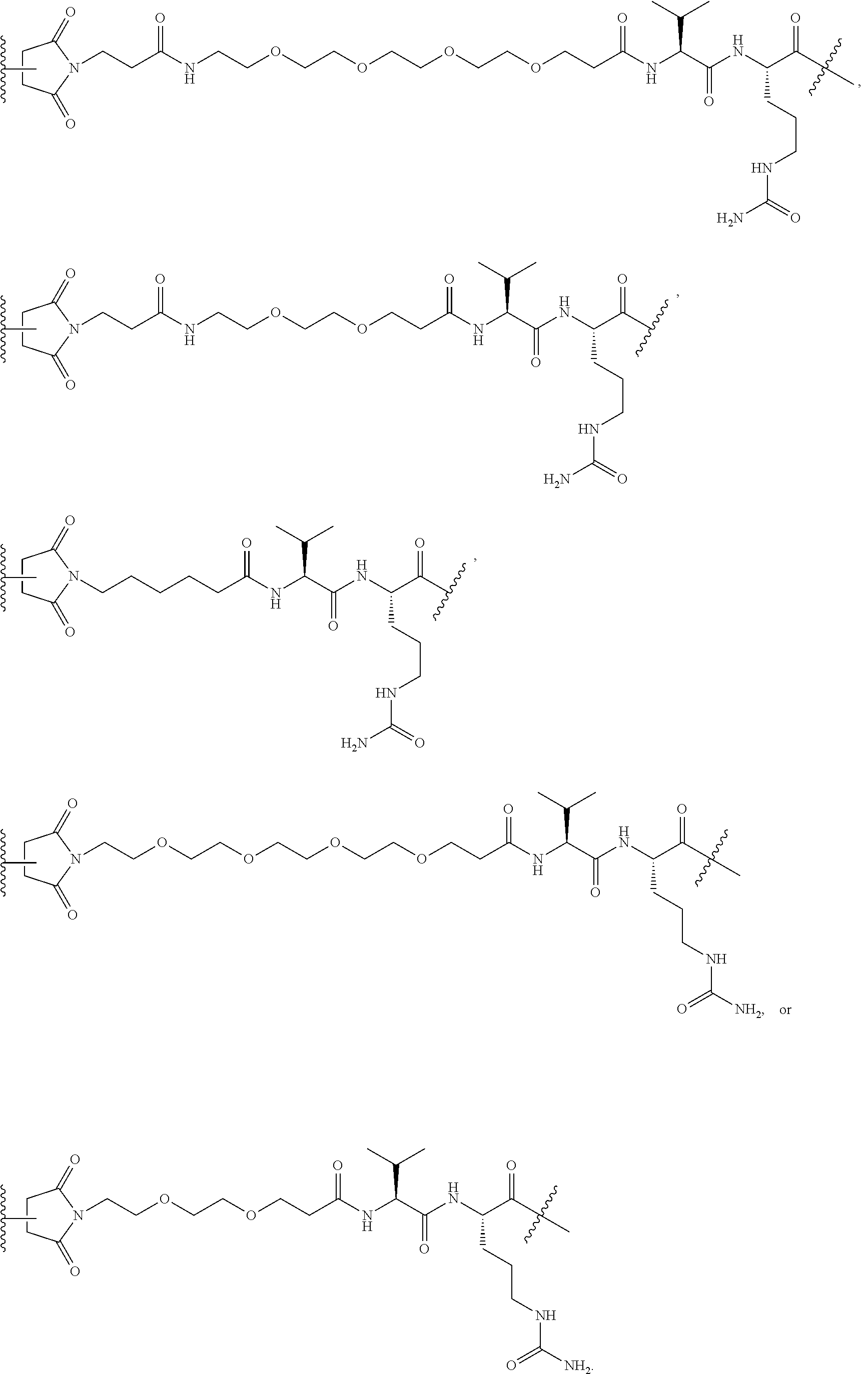

42. The compound of claim 3, wherein A-L⁴-L³-L²-X-L¹-D is:

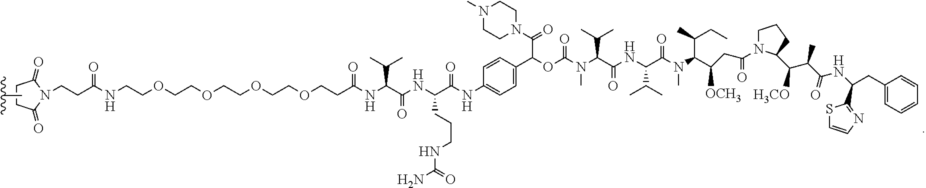

43. The compound of claim 3, wherein A-L⁴-L³-L²-X-L¹-D is:

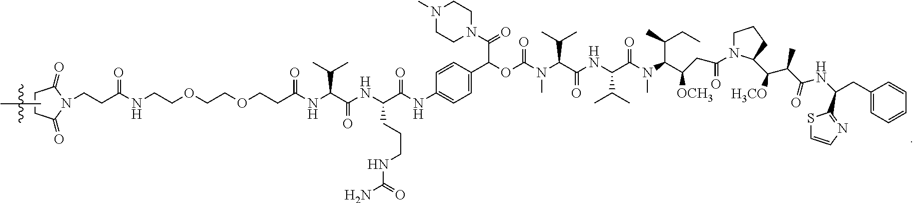

44. The compound of claim 3, wherein A-L⁴-L³-L²-X-L¹-D is:

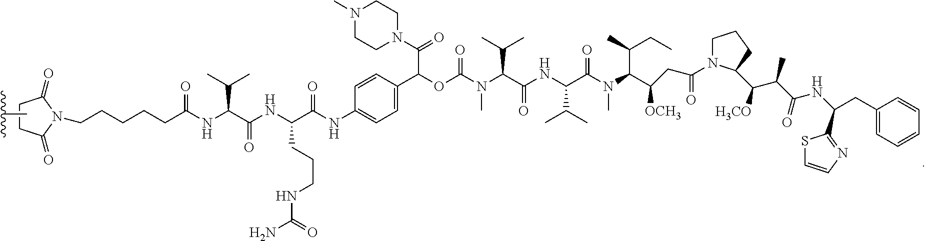

45. The compound of claim 3, wherein the anti-HER2 antibody is a humanized antibody, a chimeric antibody, a monoclonal antibody or a human antibody.

46. The compound of claim 45, wherein the humanized anti-HER2 antibody is trastuzumab, pertuzumab or margetuximab.

47-48. (canceled)

49. The compound of claim 3, wherein one or more amino acid residues of the heavy chain and/or the light chain of the antibody is replaced with a cysteine residue.

50. The compound of claim 49, wherein one or more amino acid residues of the Fc region of the antibody is replaced with a cysteine residue.

51. The compound of claim 49, wherein the one or more amino acid residues of the antibody is at position 147, 188, 200, 201 and/or 206 of the light chain, and/or at position 155, 157, 165, 169, 197, 199, 209, 211 and/or 442 of the heavy chain using EU numbering.

52. The compound of claim 49, wherein D is linked to T by way of the cysteine residue.

53. The compound of claim 3, wherein the anti-HER2 antibody comprises a heavy chain variable region and a light chain variable region, wherein
(1) the heavy chain variable region comprises the three heavy chain CDRs of the amino acid sequence of SEQ ID NO:16-18 and/or the light chain variable region comprises the three light chain CDRs of the amino acid sequence of SEQ ID NO:19-21;
(2) the heavy chain variable region comprises the three heavy chain CDRs of the amino acid sequence of SEQ ID NO:22-24 and/or the light chain variable region comprises the three light chain CDRs of the amino acid sequence of SEQ ID NO:25-27; or
(3) the heavy chain variable region comprises the three heavy chain CDRs of the amino acid sequence of SEQ ID NO:28-30 and/or the light chain variable region comprises the three light chain CDRs of the amino acid sequence of SEQ ID NO:31-33.

54. The compound of claim 3, wherein the anti-HER2 antibody comprises a heavy chain variable region and a light chain variable region, wherein
(1) the heavy chain variable region comprises the amino acid sequence of SEQ ID NO:8 and/or the light chain variable region comprises the amino acid sequence of SEQ ID NO:7;
(2) the heavy chain variable region comprises the amino acid sequence of SEQ ID NO:13 and/or the light chain variable region comprises the amino acid sequence of SEQ ID NO:12;
(3) the heavy chain variable region comprises the amino acid sequence of SEQ ID NO:15 and/or the light chain variable region comprises the amino acid sequence of SEQ ID NO:14.

55. A pharmaceutical composition comprising a compound of claim 3, or a salt or solvate or stereoisomer thereof; and a pharmaceutically acceptable carrier.

56. A method of killing a cell, comprising administering to the cell an amount of the compound of claim 3, or a salt or solvate or stereoisomer or a pharmaceutical composition thereof, sufficient to kill the cell.

57. The method of claim 56, wherein the cell is a cancer cell.

58. The method of claim 57, wherein the cancer cell is a breast cancer cell, gastric cancer cell, or ovarian cancer cell.

59. A method of treating cancer in an individual in need thereof comprising administering to the individual an effective amount of a compound of claim 3, or a salt or solvate or stereoisomer or a pharmaceutical composition thereof.

60. The method claim 59, wherein the cancer is breast cancer, gastric cancer, or ovarian cancer.

61. A kit comprising a compound of claim 1, or a salt or solvate or stereoisomer or a pharmaceutical composition thereof.

62. A method of preparing a compound of claim 2,
the method comprising reacting an antibody with Compound Z: 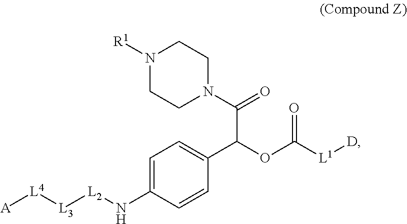
or a salt or solvate or stereoisomer thereof.

63. A method of preparing a compound of claim 4,
the method comprising reacting an antibody with Compound Z: 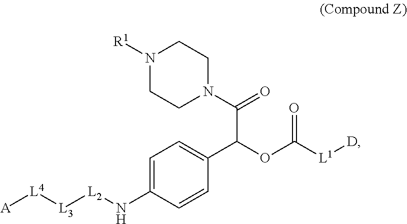
or a salt or solvate or stereoisomer thereof.

64-68. (canceled)

69. A compound, or a salt or solvate or stereoisomer thereof, wherein the compound is prepared by a method according to claim 63, wherein the antibody comprises one or more sulfhydryl groups.

70. (canceled)
